(19) United States
(12) Patent Application Publication
(10) Pub. No.: US 2021/0278391 A1
(43) Pub. Date: Sep. 9, 2021

(54) COMPOSITIONS AND METHODS FOR TREATING CARDIOVASCULAR AND METABOLIC CONDITIONS

(71) Applicant: The Regents of the University of California, Oakland, CA (US)

(72) Inventors: Yibin Wang, Encino, CA (US); Haipeng Sun, Los Angeles, CA (US)

(21) Appl. No.: 16/482,977

(22) PCT Filed: Feb. 1, 2018
(86) PCT No.: PCT/US2018/016393

§ 371 Date: Aug. 1, 2019

Related U.S. Application Data
(60) Provisional application No. 62/453,867, filed on Feb. 2, 2017.

(51) Int. Cl. G01N 33/483; A61K 31/381; A61P 3/00

(52) U.S. Cl. CPC G01N 33/4833; G01N 2560/00; A61P 3/00; A61K 31/381

(57) ABSTRACT

The present application provides a method of assessing metabolic competence and treating metabolic disorders, including cardiovascular disease, obesity, and diabetes.

### RELATED APPLICATIONS

This application claims the benefit of priority to U.S. Provisional Patent Application Ser. No. 62/453,867, filed Feb. 2, 2017, which is herein incorporated by reference in its entirety.

### GOVERNMENT SUPPORT

This invention was made with Government support under Grant Number HL108186, Grant Number HL103205, Grant Number HL098954, Grant Number HL080111, Grant Number DK62306, and Grant Number R01DK104363, awarded by the National Institutes of Health. The Government has certain rights in this invention.

### BACKGROUND OF THE INVENTION

Branched chain amino acid (BCAA), including leucine, isoleucine, and valine, are essential amino acids that share a degradation pathway. BCAA catabolism is initiated by BCAT2 which facilitates a reversible transamination reaction generating branched-chain α-keto acids (BCKA) including α-ketoisocaproic acid (KIC, from leucine), α-keto-β-methylvaleric acid (KMV, from isoleucine), and α-ketoisovaleric acid (KIV, from valine). The subsequent irreversible decarboxylation of BCKA by branched-chain alpha-keto acid dehydrogenase (BCKD) is the rate-limiting step in BCAA catabolism, giving rise to CoA moieties that feed into the citric acid cycle. In addition to substrate-dependent allosteric modulation, BCKD activity is also activated by post-translational modifications. Phosphorylation of BCKD E1α subunit by kinase BCKDK inhibits BCKD while dephosphorylation by protein phosphatase PP2Cm activates BCKD. Defects in the BCAA catabolic pathway lead to the accumulation of BCAA/BCKA (Lu et al. (2009) J. Clin. Invest. 119:1678-1687; Burrage et al. (2014) Hum. Mol. Genet. 23:R1-R8), and genetic loss of function mutations in BCAA catabolic genes lead to a metabolic disease, Maple Syrup Urine Disease, characterized by premature death and seizure in the affected infants.

In addition to serving as essential nutrients for protein synthesis, BCAA are also known to have signaling function to modulate metabolism. Indeed, BCAA supplements or BCAA-rich protein diets are often used as a dietary approach to improve glucose homeostasis and insulin sensitivity (Lynch and Adams (2014) Nat Rev Endocrinol 10:723-736). However, more recent studies highlighted a significant and strong association between elevated plasma BCAA and insulin resistance (IR) in human and rodent models of metabolic syndrome (Newgard et al. (2009) Cell Metabolism 9:311-326). Moreover, a longitudinal clinical study suggested that high plasma BCAA levels were predictive of the future onset of diabetes (Wang et al. (2011) Nat Med 17:448-453). Circulating BCAA levels are a significant prognostic marker associated with intervention outcomes of diabetes (Lu et al. (2013) Front. Med. 7:53-59; Shah et al. (2012) Diabetologia 55:321-330). White et al. recently also showed that BCAA restriction in obese rats improved muscle insulin sensitivity, suggesting a cause and effect relationship between BCAA supply and insulin sensitivity (White et al. (2016) Molecular Metabolism 5:538-551). The elevated plasma BCAA may result from impaired degradation activity and increased supply from gut microbiota. Enhancing mTOR activation and interfering with lipid oxidation have been suggested as possible mechanisms for BCAA's effect on IR. However, a complete understanding of BCAA catabolism and its relationship to insulin resistance has not yet been achieved. Methods of predicting, preventing, and/or treating metabolic disease are needed.

### SUMMARY OF THE INVENTION

The present invention relates, in part, to a discovery that BCAA defects in cardiomyocytes are associated with heart failure and BCAA defects in fat tissue are associated with obesity and diabetes. Specifically, BCAA downstream metabolites, including branched-chain keto acids and 3,3-dimethylacrylic acid (DMAA) have potent signaling effects that impact cellular viability, adipogenesis, and inflammatory status of fatty tissue. In addition, BCAA catabolic flux defects can be restored by inhibiting BCKD kinase in the pathological settings of heart failure and diabetes associated with obesity.

In one aspect, the present invention provides a method of assessing metabolic competence in a subject, comprising:

a) administering to the subject a dosage of a branched-chain amino acid (BCAA) and/or a branched-chain α-keto acid (BCKA), wherein the dosage is effective to increase the amount of the BCAA and/or BCKA in the subject;

b) after a pre-determined period of time, obtaining a sample from the subject;

c) measuring the amount of the BCAA and/or BCKA in at least one subject sample; and

d) comparing the measured amount of the BCAA and/or BCKA against a control sample or a pre-determined reference value,

wherein an increase in the amount of the BCAA and/or BCKA in the at least one subject sample relative to the control sample or pre-determined reference value indicates diminished metabolic competence in the subject.

In another aspect, the present invention provides a method of assessing metabolic competence in a subject, comprising:

a) administering to the subject a dosage of a branched-chain amino acid (BCAA) and/or a branched-chain α-keto acid (BCKA), wherein the dosage is effective to increase the amount of the BCAA and/or BCKA in the subject;

b) after a pre-determined period of time, obtaining a sample from the subject;

c) measuring the amount of the BCAA and/or BCKA in the sample;

d) repeating step b) and step c) until the measured amount of the BCAA and/or BCKA in the subject sample is no more than a pre-determined amount;

e) determining the length of a response time period from the administration in step a) to when the measured amount of the BCAA and/or BCKA in the subject sample is no more than the pre-determined amount; and

f) comparing the response time period to a response time period for a control sample or a pre-determined reference value,

wherein an increase in the measured total time period for the subject sample relative to that for the control sample or pre-determined reference value indicates diminished metabolic competence in the subject.

In some embodiments, the pre-determined reference value of the BCAA and/or BCKA described herein is a normal physiological level of BCAA and/or BCKA.

In some embodiments, the control sample or pre-determined reference value described herein is a measurement of a previous sample or reference sample obtained from the subject prior to the administration in step a) as described herein.

In some embodiments, in step c) described herein, the at least one subject sample is collected after the amount of the BCAA and/or BCKA reaches its peak amount in the subject after the administration.

In another aspect, the present invention provides a method of prognosing or predicting metabolic competence in a subject, comprising:

a) administering to the subject a dosage of a branched-chain amino acid (BCAA) and/or a branched-chain α-keto acid (BCKA), wherein the dosage is effective to increase the amount of the BCAA and/or BCKA in the subject;

b) after a pre-determined period of time, obtaining a sample from the subject;

c) measuring the amount of the BCAA and/or BCKA in the sample;

d) comparing the measured amount of the BCAA and/or BCKA in the sample to a control sample or a pre-determined reference value;

wherein an increase in the measured amount of the BCAA and/or BCKA in the at least one subject sample relative to the control sample or pre-determined reference value indicates a positive prognosis for glucose intolerance in the subject. In some embodiments, in step c) described herein the at least one subject sample is collected after the amount of the BCAA and/or BCKA reaches its peak amount in the subject after the administration.

In another aspect, the present invention provides a method of assessing the efficacy of an agent for treating a metabolic disorder in a subject, comprising:

a) measuring the levels of a branched-chain amino acid (BCAA) and/or a branched-chain α-keto acid (BCKA) in a first subject sample maintained in the absence of the agent;

b) measuring the levels of the BCAA and/or BCKA in at least one subsequent subject sample contacted with the agent or maintained in the presence of the agent; and

c) comparing the amounts of BCAA and/or BCKA from steps a) and b),

wherein a decrease in the amount in b) relative to the amount in a) indicates that the agent treats the metabolic disorder in the subject.

In another aspect, the present invention provides a method of assessing the efficacy of an agent for treating a metabolic disorder in a subject, comprising:

a) measuring the amount of a branched-chain amino acid (BCAA) and/or a branched-chain α-keto acid (BCKA) in a first subject sample contacted with the agent or maintained in the presence of the agent;

b) repeating step a) to measure the amount of the BCAA and/or BCKA in at least one subsequent subject sample; and

c) comparing the amounts of BCAA and/or BCKA from step a) and b),

wherein a decrease in the measured amount of BCAA and/or BCKA in the at least one subsequent subject sample relative to the first subject sample indicates that the agent treats the metabolic disorder in the subject.

In some embodiments, the first and/or at least one subsequent sample described herein is a portion of a single sample or pooled samples obtained from the subject.

In some embodiments, the at least one of the subject samples is an ex vivo or in vivo sample.

In some embodiments, the methods described herein further comprise recommending, prescribing, or administering to the subject a therapeutic agent that specifically antagonizes the BCKD kinase (BCKDK).

In some embodiments, the methods described herein further comprise recommending, prescribing, or administering a BCAA-deficient diet to the subject.

In another aspect, the present invention provides a method for treating or preventing a metabolic disorder in a subject, comprising administering to the subject a therapeutically effective amount of at least one agent capable of antagonizing BCKDK, to thereby treat or prevent the metabolic disorder in the subject. In some embodiments, the method further comprises administering to the subject an additional agent and/or therapy to treat or prevent the metabolic disorder in the subject. In some such embodiments, the additional therapy comprises a BCAA-deficient diet.

In some embodiments, the subject described herein has glucose intolerance.

In some embodiments, the subject has insulin resistance.

In some embodiments, the subject has obesity and/or a cardiovascular disease, optionally wherein the cardiovascular disease is heart failure.

In some embodiments, the agent described herein antagonizes the expression and/or function of the BCKD kinase (BCKDK).

In some embodiments, the agent increases the expression and/or function of branched-chain alpha-keto acid dehydrogenase (BCKD).

In some embodiments, the agent increases the expression and/or function of Sfrp5.

In some embodiments, the agent is a polynucleotide, a polypeptide, a small molecule compound, or a derivative and/or fragment thereof, such as an mRNA or a cDNA. In some embodiments, the agent is RNA interfering agent, such as siRNA, shRNA, microRNA, CRISPR gRNA, and piwi.

In some embodiments, the agent is a fusion protein. For example, the agent may be an antibody or antigen-binding fragment thereof, such as anti-BCKDK antibodies or fragments thereof.

In some embodiments, the agent is a small molecule compound that inhibits BCKDK, such as BT-2.

In some embodiments, the subject described herein is a mammal, such as a human or a non-human mammal.

### BRIEF DESCRIPTION OF THE DRAWINGS

FIGS. 1A-1D depict remodeling of branched-chain amino acid (BCAA) catabolism in murine failing heart. FIG. 1A shows downregulated genes in failing heart mapped into BCAA catabolism pathway by Kyoto Encyclopedia of Genes and Genomes. FIG. 1B shows the real-time reverse transcription-polymerase chain reaction result of specific genes using mRNA from myocardium of neonatal (n=3), normal (adult sham; n=3), and failing (adult failing; n=3) mouse hearts. The y axis represents the relative mRNA level. ANOVA followed by the Newman-Keuls test was performed. *P<0.05 vs neonatal; #P<0.05 vs adult sham. FIG. 1C shows Western blotting result of proteins involved in BCAA catabolism (GAPDH as loading control) using tissue lysates from 3 individual normal (sham) or failing mouse hearts (n=3). FIG. 1D shows individual branched-chain α-keto acid (BCKA) concentration in tissues from normal (sham, n=9) and failing (n=7) mouse hearts. Error bars represent SD (B) or SEM (D). *P<0.05, **P<0.01.

FIGS. 2A-2B depict impaired branched-chain amino acid (BCAA) catabolism in human failing heart. FIG. 2A shows real-time reverse transcription-polymerase chain reaction result of specific genes using mRNA from myocardium of control (Ctrl; n=4) and failing (failure; n=11-15) human hearts. They axis shows the relative mRNA level. FIG. 2B shows individual branched-chain α-keto acid (BCKA) concentration in tissues from control (n=3) and failing (n=10) human hearts. Error bars represent SEM. BCKDK indicates BCKD kinase. *P<0.05.

FIGS. 3A-3D show that Krüppel-like factor 15 (KLF15) regulates branched-chain amino acid (BCAA) catabolic gene expression. FIG. 3A shows real-time reverse transcription-polymerase chain reaction result of specific genes using mRNA from neonatal rat ventricular myocytes with (KLF15) or without (vector) KLF15 overexpression (n=6). *P<0.05 vs vector control. FIG. 3B shows Western blotting result of proteins involved in BCAA catabolism (GAPDH as loading control) using cellular lysates from KLF15-overexpressed Hela cells. FIG. 3C shows an illustration of partial mouse PP2Cm promoter fragments with 2 GC-rich sites and luciferase assay result of PP2Cm promoter-luciferase in HeLa cells cotransfected with either KLF15 or corresponding empty vector. The data represented the average values, with the SD of triplicate samples from 1 experiment representative of 3 independent experiments. *P<0.05 vs same promoter without KLF15 overexpression; #P<0.05 vs 468-bp promoter with KLF15 overexpression (n=3). FIG. 3D shows representative result of chromatin immunoprecipitation-polymerase chain reaction validation for KLF15 binding to the PP2Cm gene promoter in neonatal rat ventricular myocytes after KLF15 overexpression. The experiment was repeated twice with similar results.

FIGS. 4A-4C show that ablation of cardiac Krüppel-like factor 15 (KLF15) downregulates branched-chain amino acid (BCAA) catabolism. FIG. 4A and FIG. 4B show real-time reverse transcription-polymerase chain reaction (FIG. 4A) and Western blotting (FIG. 4B) result of specific genes in wild-type (WT; n=4) and KLF15-deficient (KLF15 KO; n=4) hearts. FIG. 4C shows the level of branched-chain α-keto acids (BCKAs) in WT (n=4) and KLF15 deficient (n=5) heart. Error bars represent SD (FIG. 4A) or SEM (FIG. 4C). *P<0.05 vs wild-type.

FIGS. 5A-5G show that branched-chain amino acid (BCAA) catabolic defect impairs cardiac function but not structure. FIG. 5A and FIG. 5B show individual branched-chain α-keto acid (BCKA) concentrations in cardiac tissue of PP2Cm germ-line knockout mice (KO) and wild-type (WT) mice. FIG. 5A depicts mice on a normal chow (20% protein) were fasted for 6 hours (WT, n=9; KO, n=10). FIG. 5B shows mice fasted overnight and fed with a high-protein diet (40% protein) for 2 hours (WT, n=5; KO, n=5). Error bars represent SEM. **P<0.01 vs WT. FIG. 5C and FIG. 5D show left ventricular ejection fraction (LV % EF) from WT and PP2Cm-KO mice at 3 months (in FIG. 5C); WT, n=12; KO, n=15) or 18 months (in FIG. 5D); n=5 in each group) of age. FIG. 5E shows the morphology of hearts from WT and PP2Cm-KO mice. FIG. 5F shows a longitudinally sectioned heart stained with hematoxylin and eosin. Magnification ×200. FIG. 5G shows transmission electron microscopy used in hearts from PP2Cm-KO and WT mice. Magnification ×7400.

FIGS. 6A-6D show that branched-chain amino acid (BCAA) catabolic defect promotes heart failure progression. FIG. 6A and FIG. 6B show the time course for left ventricular fractional shortening (LV % FS; in A)) and left ventricular internal dimension (in millimeters) at systole (LVIDs; in B)) from wild-type (WT; n=11-15) and PP2Cm germ-line knockout (PP2Cm-KO) mice (n=13-19) with transaortic constriction (TAC) surgery. The x axis shows the time in weeks after surgery. FIG. 6C and FIG. 6D show representative M-mode echocardiographs (in FIG. 6C)) or ratio of lung weight to body weight (LW/BW; in FIG. 6D); WT sham, n=9; KO sham, n=10; WT TAC, n=8; KO sham, n=8) from WT and PP2Cm-KO mice at 8 weeks after surgery. Error bars represent SEM. Statistical analyses were performed with the Student t test (in FIG. 6A) and FIG. 6B)) to compare the values of WT and PP2Cm-KO at the same time point (#P=0.05, *P<0.05) or the Kruskal-Wallis test followed by the Dunn multiple-comparison test (in D); *P<0.05 vs KO sham). A repeated-measures linear model was also fitted for LVIDs (in FIG. 6A)) and LV % FS (in FIG. 6B)).

FIGS. 7A-7H depict disturbed metabolic and redox homeostasis by branched-chain α-keto acids (BCKAs). FIG. 7A shows oxygen consumption in mitochondria isolated from wild-type (WT) hearts in the absence or presence of 500 μmol/L BCKAs. FIG. 7B shows relative oxygen consumption rate in the absence or presence of BCKAs at different concentrations (n=3-8 in each group; *P<0.05 vs control). FIG. 7C shows superoxide production in isolated cardiac mitochondria (n=4-7 in each group; *P<0.05 vs control). FIG. 7D and FIG. 7E shows superoxide production in isolated mitochondria (in FIG. 7D), n=5-6 in each group) and myocardium (in FIG. 7E), n=3 in each group) from WT and PP2Cm germ-line-deficient (KO) mice. FIG. 7F shows immunoblotting of total protein oxidation detected by carbonyl groups (left) from tissue lysates of WT and PP2Cm-KO mouse hearts. FIG. 7G shows principal component analysis of metabolomic profiles revealed a distinct genotype-based separation for the heart samples (WT, n=8; KO, n=7). FIG. 7H shows a list of the top 30 biochemicals that separated different genotypes based on their importance. Error bars represent SEM (in FIG. 7B-FIG. 7E)).

FIGS. 8A-8G show that inhibition of BCKD kinase (BCKDK) by BT2 (3,6-dichlorobenzo[b]thiophene-2-carboxylic acid) promotes branched-chain α-keto acid (BCKA) degradation and preserves cardiac function in the pressure-overloaded heart. FIG. 8A shows an immunoblot for total and phosphorylated BCKD subunit E1α in heart from wild-type (WT) mice treated with vehicle (veh; n=4) or BT2 (n=5). FIG. 8B shows the average phosphorylation level of E1α vs total E1α presented with the SEM. Error bars represent SEM. *P<0.05 between vehicle- and BT2-treated samples. FIG. 8C shows the BCKD activity in cardiac tissues from WT or PP2Cm germ-line knockout mice (KO) mice treated with vehicle or BT2 (n=4-5 in each group). *P<0.05, vehicle- vs BT2-treated groups. FIG. 8D shows the individual BCKA concentration in plasma from WT and PP2Cm-KO (n=4-6) mice treated with vehicle or BT2. *P<0.05, vehicle- vs BT2-treated groups of the same genotype. FIG. 8E shows the representative M-mode echocardiographs of mouse hearts after sham surgery or transaortic constriction (TAC) treated with vehicle or BT2. FIG. 8F shows the left ventricular ejection fraction (% LVEF; n=6-8) and G) left ventricular internal dimension at systole (LVIDs; n=6-8) from mice with sham or TAC surgery for 4 weeks, treated with or without BT2 as indicated. Error bars represent SEM. *P<0.05 between designated groups.

FIGS. 9A-9B depict the densitometric values of the bands in FIG. 1C (in FIG. 9A) and the western blotting result of BCAA catabolic enzymes with GAPDH as loading control (in FIG. 9B). The densitometric value of each protein was normalized to GAPDH and presented as fold change versus Sham (n=3 in each group). The data represented the average values with standard deviation of three bands with p value labeled. The densitometric value of phosphorylated BCKDE1a was compared to that of total BCKDHE1a and the ratio was shown on top of the panels.

FIG. 10 depicts individual BCAA concentration in tissues from normal (Sham, n=10) and failing (Failure, n=7) murine hearts was measured and normalized to the weight of tissue. Error bars represent SEM.

FIG. 11 depicts individual BCKA concentration in plasma from human with normal (Ctrl, n=50) or failing (Failure, n=91) hearts was measured. Error bars represent SEM. *, p<0.05 compared to control.

FIG. 12 depicts individual BCAA concentration in tissues from normal (Ctrl, n=3) and failing (Failure, n=9) human hearts was measured and normalized to the weight of tissue. Error bars represent SEM.

FIGS. 13A-13B depict the real-time RT-PCR results of genes using mRNA from HeLa cells with (KLF15, n=3) or without (Vector, n=3) KLF15 overexpression (in FIG. 13A) and the luciferase assay result of PP2Cm promoter-luciferase in HeLa cells co-transfected with either KLF15 or corresponding empty vector (in FIG. 13B). In FIG. 13A, the Y axis represents relative mRNA level. The data represented the average values with standard deviation of three samples. *, p<0.05 compared to vector control. In FIG. 13B, 486 bp, the 486 bp promoter of PP2Cm; 486bpDD, the 486 bp promoter with two GC-rich sites deleted. The data represented the average values with standard deviation of triplicate samples from one experiment representative of three independent experiments. *, p<0.05 compared to same promoter without KLF15 overexpression. #, p<0.05 compared to 468 bp promoter with KLF15 overexpression.

FIGS. 14A-14B depict the densitometric values of the bands in FIG. 4B (in FIG. 14A) and the real-time RT-PCR result of KLF15 gene using mRNA, normalized to 18sRNA, from normal (Sham, n=3) and failing (Failure, n=3) heart induced by pressure overload (in FIG. 14B). In A), the densitometric value of each protein was normalized to GAPDH. The data represented the average values of relative densitometry with standard deviation of four hearts. *, p<0.05 compared to WT control. In FIG. 14B, the data represented the average values with standard deviation of three hearts. *, p<0.05 compared to control.

FIGS. 15A-B depict individual BCAA concentration in tissues from PP2Cm knockout (KO, n=5) and wild type (WT, n=5) mouse heart, measured with mass spectrometer and normalized to weight of tissue (in FIG. 15A), and gene expression examined by microarray using RNA from PP2Cm knockout (KO, n=3) and wild type (WT, n=3) mouse heart FIG. 15B. Error bars represent SEM. *, p<0.05. In FIG. 15B), the data was presented as fold change versus WT. *, p<0.05.

FIG. 16A depicts the time course for left ventricular internal dimension at diastole (LVIDd) from TAC WT (n=11-15) and PP2Cm KO mice (n=9-13). The X-axis show the time in weeks after surgery. *, p<0.05 compared to WT.

FIG. 16B depicts the time course for left ventricular posterior wall thickness at diastole (LVPWd) from TAC WT (n=11-15) and PP2Cm KO mice (n=9-13). The X-axis show the time in weeks after surgery. *, p<0.05 compared to WT.

FIG. 16C depicts the time course for left ventricular posterior wall thickness at systole (LVPWs) from TAC WT (n=11-15) and PP2Cm KO mice (n=9-13). The X-axis show the time in weeks after surgery. *, p<0.05 compared to WT.

FIG. 16D depicts the time course for Ejection Fraction (% EF) from TAC WT (n=11-15) and PP2Cm KO mice (n=9-13). The X-axis show the time in weeks after surgery. *, p<0.05 compared to WT.

FIGS. 17A-17B depict the oxygen consumption in mitochondria isolated from wildtype hearts in absence or presence of BCKA-Na (500 μM each of KIC, KIV, KMV mixed) (FIG. 17A) and the relative oxygen consumption rate in the absence or presence of BCKA calculated based on results in FIG. 17A) (n=3 in each group) (FIG. 17B). In FIG. 17A, NaCl (1.5 mM) was used as control. Y axis: oxygen concentration (ppm) in assay buffer. The assay was completed in ˜12 minutes.

FIGS. 18A-18B show that deletion of PP2Cm resulted in significant global perturbations in cardiac metabolism. FIG. 18A shows a Random Forest Confusion Matrix. Random Forest classification using named metabolites detected in heart tissue of wild type and PP2Cm KO mice resulted in a predictive accuracy of 100%. FIG. 18B shows metabolomic analysis in PP2Cm deficient heart compared to wild type which showed higher level of glucose, glycolytic intermediates, glucose-derived sugars such as fructose, and malate in PP2Cm deficient heart (right, n=7) compared to that in wild type heart (left, n=8) at baseline. Statistical analyses were performed with Welch's two-sample t-test. p<0.05 for all shown compounds.

FIG. 19 shows that inhibition of BCKDK by BT2 reduced plasma BCAA level. Individual BCAA concentration in plasma from wild type and PP2Cm-KO (n=4-6) mice treated without (vehicle, veh group) or with BT2 (BT2 group). Error bars represent SEM. Statistical analyses were performed with Student's t-test to compare the values of two groups. *, p<0.05 compare to KO Veh group.

FIGS. 20A-20C show that inhibition of BCKDK by BT2 preserves cardiac function. FIG. 20A shows the left ventricular internal dimension at diastole (LVIDd). FIG. 20B and FIG. 20C shows the left ventricle pastier wall thickness at systole (LVPWs, in B)) and diastole (LVPWd, in FIG. 20C)) from mice following sham or post-TAC surgery at 4 weeks, treated with or without BT2 (n=6-8 in each group). Error bars represent SEM. Statistical analyses were performed with One-Way ANOVA followed by Newman-Keuls test for FIG. 20A and FIG. 20B, or Kruskal-Wallis test followed by Dunn's multiple comparison test for FIG. 20C). *, p<0.05 between two groups.

FIGS. 21A-21G show that BCAA catabolic defect in obese mice is characterized by BCKD deficiency. FIG. 21A shows an illustration of BCAA catabolic process with enzymes, intermediates, and derivatives. FIG. 21B shows the relative level of BCAA and their metabolites in plasma of lean wild type (n=6) and ob/ob (n=8) mice (age of 14 weeks) with 6-hour fasting. FIG. 21C shows the plasma levels of BCAA and BCKA upon i.p. administration of leucine in lean wild type (n=6) and ob/ob (n=6) mice (age of 10 weeks) with 6-hour fasting. FIGS. 21D-FIG. 21F shows the Relative fasting levels of BCAA and their metabolites in white adipose tissue (FIG. 21D), skeletal muscle (FIG. 21E), and liver (FIG. 21F) in lean wildtype (n=6) and ob/ob (n=8) mice at age of 14 weeks. FIG. 21G shows the Western blot analysis of BCAA catabolic enzymes in different tissues of wild type (n=4) and ob/ob (n=4) mice. GAPDH was used as a loading control for western blots. Data are presented as mean±SEM. *p<0.05, **p<0.01, compared to lean mice.

FIGS. 22A-22F show BCAA catabolic defect promotes insulin resistance in obese mice. Ob/ob mice were fed a normal chow diet (NCD, 20% protein by weight) or low protein diet (LPD, 6% protein by weight) beginning at 10 weeks of age for 4 weeks. For BCAA group (LPD+BCAA), supplement of BCAA in drinking water (3 mg/ml) was started after LPD for 2 weeks and lasted for 2 weeks. Plasma levels of BCAA and metabolites (in FIG. 22A), n=8, *p<0.05 compared to NCD, &p<0.05 compared to LPD), glucose tolerance test results (in FIG. 22B-FIG. 22C), n=6, *p<0.05, **p<0.01 compared to NCD, &p<0.05, &&p<0.01compared to LPD), plasma insulin levels (in FIG. 22D), n=8-12, *p<0.05, &p<0.05), food intake (in FIG. 22E), n=6), and body weight (in FIG. 22F), n=14, **p<0.01) were analyzed. Data are presented as mean±SEM.

FIGS. 23A-23D show that increasing BCAA uptake impairs insulin sensitivity in a tissue-specific but mTOR- and BCAA-independent pattern. FIG. 23A shows the insulin tolerance test result of ob/ob mice fed on low protein diet (LPD) without or with BCAA supplement (LPD/AA) (n=6 in each group, *p<0.05, **p<0.01 compared to LPD). FIG. 23B shows representative immunoblots for specific proteins using tissue lysates from skeletal muscle, white adipose tissue, and liver of mice without (LPD) or with BCAA supplement (LPD/AA), without or with (+) insulin injection (n=4 in each group). FIG. 23C shows the relative abundance of BCAA detected by metabolomics analysis in skeletal muscle (SM), liver, and white adipose tissue (WAT) of ob/ob mice without (LPD) or with BCAA supplement (LPD+BCAA) (n=8 in each group *p<0.05). FIG. 23D shows the relative abundance of acylcarnitines detected by metabolomics analysis in skeletal muscle of ob/ob mice without (LPD) or with BCAA supplement (LPD+BCAA) (n=8 in each group *p<0.05). Data are presented as mean±SEM.

FIGS. 24A-24D shows that integrative genomic analyses associate adipose BCAA metabolic pathway with IR-related traits in human and mouse populations. FIG. 24A shows the integrative genomics workflow used to investigate the association of BCAA with IR-related traits in human. Specifically, human GWAS were integrated with eQTLs and co-expression networks matched by tissue, and analyzed using Mergeomics pipeline to identify co-expression modules that show significant genetic association with IR-related clinical traits. BCAA modules were then retrieved based on significant over-representation of BCAA genes among the module genes. FIG. 24B shows the number of BCAA modules with significant trait association (FDR<5% or p<0.05 assessed by Marker Set Enrichment Analysis). Numbers in the bar indicate fold enrichment of BCAA modules among all significant co-expression modules for the corresponding trait. Statistical significance of enrichment of BCAA modules among all significant modules is determined by Fisher's exact test, *p<0.05, **p<0.01, ***p<0.001, ****p<0.0001. FIG. 24C shows the comparison of tissue origin distribution of BCAA modules and all co-expression modules significantly associated with fasting insulin and insulin resistance (BMI unadjusted) at FDR<5% and p<0.05. FIG. 24D shows the comparison of correlation strengths of BCAA genes, non-BCAA amino acids pathway genes and all genes with IR-related traits in mouse. The correlation data between tissue-specific expression profiling and clinical traits measurements were extracted from HMDP which contains ˜100 strains of genetically divergent mice fed with a high-fat diet. The average and standard error of the absolute values of Pearson correlations between each gene within a gene category (BCAA, non-BCAA, all gene) and a trait are shown. Significance of differences in the average correlation strength between gene categories is calculated using the Student's t-test. *p<0.05, **p<0.01, ***p<0.001, ****p<0.0001.

FIGS. 25A-25G shows that Adipokine Sfrp5 mediates the effect of BCAA catabolic defect on insulin resistance. FIG. 25A shows the real-time RT-PCR result of Sfrp5 using mRNA from adipose tissue of wild type (WT) or PP2Cm knockout (KO) mice on 20% protein normal chow diet (NCD), 40% high protein diet (HPD), or 6% low protein diet (LPD). n=6 in each group. FIG. 25B shows the real-time RT-PCR result of Sfrp5 using mRNA from differentiated 3T3-L1 adipocytes with or without BCAA (800 μM)/BCKA (500 μM) treatment. FIG. 25C shows the real-time RT-PCR result of Sfrp5 using mRNA from adipose tissue of ob/ob mice on normal chow diet (NCD), low protein diet (LPD), or LPD with supplement of BCAA in drinking water (LPD+BCAA). n=6 in each group. FIGS. 25D-25G show Ob/ob mice fed on low protein diet (LPD) with a supplement of BCAA and ectopic expression of Sfrp5 or GFP via systemic delivery of expressing adenovirus. n=6 in each group. Real-time RT-PCR result of Sfrp5 using mRNA from adipose tissue (in FIG. 25D)), Glucose tolerance test results with LPD+GFP group (in FIG. 25E), n=6 in each group, *p<0.05 comparing LPD+BCAA+GFP group with LPD+BCAA+Sfrp5 group), plasma insulin level (in FIG. 25F), n=8 in each group), and plasma TNFα level (in FIG. 25G), n=8 in each group). *p<0.05. Data are presented as mean±SEM.

FIGS. 26A-26H depict that chemical inhibition of BCKDK corrects BCAA catabolic defect and attenuates insulin resistance in obese mice. FIGS. 26A-26E show the analysis of ob/ob mice treated with vehicle (Control) or BT2 (40 mg/kg/day) for 10 weeks by oral gavage (n=6 in each group): BCKD activities in different tissues (FIG. 26A), plasma levels of BCAA and BCKA (FIG. 26B), glucose tolerance test results (FIG. 26C), insulin tolerance test results (FIG. 26D), and plasma insulin level (FIG. 26E). FIGS. 26F-26H show the analysis of high fat diet (HFD)-induced obese mice (DIO) treated with vehicle (Control) or BT2 (BT2, 40 mg/kg/day) for 8 weeks (n=7 in each group): glucose tolerance test results (FIG. 26F), insulin tolerance test results (FIG. 26G), and plasma insulin level (FIG. 26H). *p<0.05, **p<0.01, ***p<0.005, ****p<0.0001. Data are presented as mean±SD for BCKDC activity assay and metabolite measurements; mean±SEM for tolerance tests.

FIGS. 27A-27B depict ob/ob mice body weight (FIG. 27A) and glucose tolerance test result (FIG. 27B) at age of 14 weeks. *p<0.05.

FIG. 28A-28B depict the plasma level of BCAA and BCKA in high-fat-diet-induced obese mice during leucine challenge (BCAA tolerance test). FIG. 28A shows the body weight of wild type mice. High fat diet (HFD, 60% kcal % fat) or control diet (NCD, 10% kcal % fat) (Research Diets, New Brunswick, N.J.) was used to feed C57BL/6 male mice at 6 weeks of age for 8 weeks. FIG. 28B shows the plasma levels of BCAA and BCKA. *p<0.05, **p<0.01.

FIG. 29 depicts the plasma levels of isoleucine and valine and their keto acids in ob/ob mice during leucine challenge (BCAA tolerance test). *p<0.05, **p<0.01.

FIG. 30 depicts number of BCAA genes as key drivers in the BCAA modules associated with fasting insulin and/or insulin resistance traits. Numbers in the bars indicate fold enrichment of BCAA genes among all significant key drivers at FDR<1%. Statistical significance of enrichment is determined by Fisher's exact test, *p<0.05, **p<0.01, ***p<0.001, ****p<0.0001.

FIG. 31 depicts the correlation of BCAA genes with fasting glucose, fasting insulin and HOMA-IR in HMDP mice fed with high-fat diet. Color indicates the direction of association and association strength. *p<0.05, **p<0.05 after Bonferroni correction for the number of genes and traits

FIGS. 32A-32B show that BT2 treatment slightly reduced body weight gain (FIG. 32A), but did not affect food intake (FIG. 32B) in ob/ob mice.

FIG. 33A-33C depict that BT2 improves BCKD activities in different tissues (n=5-6 in each group) (FIG. 33A), reduces BCAA (FIG. 33B) and BCKA (FIG. 33C) levels in plasma of high-fat-diet-induced obese (DIO) mice (n=7-9). DIO mice were treated with or without BT2 (40 mg/kg/day) for 8 weeks by oral gavage.

FIG. 34 depicts a schematic timeline and experimental design. C57BL/6J adult male mice (n=21) were subjected to transverse aortic constriction (TAC), and echocardiography was performed to confirm left ventricular contractility and cardiac dysfunction. Mice were then intragastrically administered a BCKDK inhibitor BT2 (n=9) or vehicle (n=8) for 6 weeks and cardiac function was assessed every week.

FIGS. 35A-35D depict a pharmacological inhibition of BCKDK by BT2 enhanced cardiac BCAA catabolic activities post-TAC. Mice were subjected to sham or TAC operation, 16 days post-surgery they were subsequently treated with BT2 or vehicle. After 6 weeks administration, (FIG. 35A) expression of total, phosphorylated BCKD subunit E1α in mouse heart were determined by Western blot. (FIG. 35B) The average phosphorylated E1α level was normalized by total E1α level. Cardiac BCAA (FIG. 35C) and plasma BCAA (FIG. 35D) were assayed as Methods described. ***, p<0.001, data presented as mean±SEM.

FIGS. 36 A-E show that inhibition of BCKDK by BT2 preserves systolic function in pressure-overloaded mouse heart. Mice were subjected to sham and TAC procedures and followed for a 2 week-period to provoke cardiac dysfunction. Then, animals were randomized to treatment with BT2 (n=9) or vehicle (n=8) at day 16 post-TAC. Mean left ventricular fractional shortening (FS) (FIG. 36A) and ejection fraction (EF) (FIG. 36B) for both groups are presented, which shows no significant differences before treatment. In following 6 weeks treatment, BT2 therapy led to significant conserved systolic performance characterized by ΔFS (FIG. 36C) and AEF which were normalized by EF and FS at day 16 respectively (FIG. 36D). Representative M-mode echocardiographs of mouse hearts following post-sham or TAC treated with vehicle or BT2 are presented (FIG. 36E). *, p<0.05, data presented as mean±SEM.

FIGS. 37A-37D show that BT2 therapy alleviates pressure-overload induced cardiac structural remodeling. After 6 weeks BT2 or vehicle treatment following 2 weeks TAC procedures, mean changes of left ventricular internal diameter (LVID) at systole (FIG. 37A) and diastole (FIG. 37B) compared with corresponding parameters at the initial time of treatment (day 16). Average left ventricular volume at systole (FIG. 37C) and diastole (FIG. 37D) obtained from echocardiographic assay was showed. *, p<0.05, data presented as mean±SEM.

FIGS. 38A-B show a table of number of survivals of BT2 or vehicle treated mice (FIG. 38A) and a Kaplan-Meier survival plot showing percent survival of BT2 and vehicle treated mice during 8 weeks post-TAC (FIG. 38B).

FIGS. 39A-39I show the development of a pharmacologically treated mouse model with existing cardiac dysfunction. FIG. 39A shows a schematic timeline and experimental design. C57BL/6J adult male mice (n=21) were subjected to transverse aortic constriction (TAC), and echocardiography was performed to confirm left ventricular contractility and cardiac dysfunction. Mice were then intragastrically administered a BCKDK inhibitor BT2 (n=9) or vehicle (n=8) for 6 weeks and cardiac function was assessed every week. Mice were subjected to TAC procedure to provoke hypertrophy and contractile dysfunction. Mean left ventricular ejection fraction (EF) (FIG. 39B) showed dramatically decrease after TAC. Left ventricular mass (LV Mass) (FIG. 39C) significantly is presented which shows significant increase during the observed period of time. And average left ventricular internal diameter at systole (FIG. 39D) and LV volume at systole (FIG. 39E) obtained from echocardiography are showed. FIG. 39F shows the expression of total, phosphorylated BCKD subunit E1α and BCKDK in mouse heart were determined by Western blot. FIG. 39G shows the average of phosphorylated E1α level that was normalized by total E1α level, average BCKDK level was normalized by GAPDH. Cardiac BCAA (FIG. 39H) and BCKA (FIG. 39I) were assayed as Methods described. ***, p<0.001, **, p<0.01, *, p<0.05 data presented as mean±SEM.

FIGS. 40A-40G show the effects of BT2 treatment on systolic function in pressure-overloaded mouse heart. Mice were subjected to sham and TAC procedures and followed for a 2 wk-period to provoke cardiac dysfunction. Then, animals were randomized to treatment with BT2 (n=9) or vehicle (n=8) at week 2 post-TAC. Mean left ventricular fractional shortening (FS) (A) and ejection fraction (EF) (FIG. 40B) for both groups are presented, which shows no significant differences before treatment. In following 6 weeks treatment, BT2 therapy led to significant conserved systolic performance characterized by ΔFS (FIG. 40C) and ΔEF which were normalized by EF and FS at week 2 respectively (FIG. 40D). *, p<0.05, data presented as mean±SEM. FIG. 40E shows heart weight to tibia length (HW/TL) ratio at week 8 post-sham or post-TAC were presented. Markers of the fetal gene program ANF (FIG. 40F) and BNP (FIG. 40G) were quantified by qRT-PCR.

FIGS. 41A-41L show BCKDK inhibitory therapy effects on myocardium contractility. Mice were subjected to sham and TAC procedures and followed for a 2 wk-period to provoke cardiac dysfunction. Then, animals were randomized to treatment with BT2 (n=9) or vehicle (n=8) at week 2 post-TAC. Mean and changes of global longitudinal strain (GLS) (FIG. 41A and FIG. 41D) and strain rate (GLSR) (FIG. 41G and FIG. 41J), global radial strain (GRS) (FIG. 41B and FIG. 41E) strain rate (GRSR) (FIG. 41H and FIG. 41K) and global circumferential strain (GCS) (FIG. 41C and FIG. 41F) and strain rate (GCSR) (FIG. 41I and FIG. 41L) for both groups are presented, which shows in following treatment period, BT2 therapy led to significant increased myocardial contractility. ***, p<0.001, **, p<0.01, *, p<0.05, data presented as mean±SEM.

FIGS. 42A-42H show BCKDK inhibitory therapy effects on myocardial wall motion. Mice were subjected to sham and TAC procedures and followed for a 2 wk-period to provoke cardiac dysfunction. Then, animals were randomized to treatment with BT2 (n=9) or vehicle (n=8) at week 2 post-TAC. Mean and changes of systolic longitudinal velocity (FIG. 42A and FIG. 42C), systolic radial velocity (FIG. 42B and FIG. 42D) and longitudinal displacement (FIG. 42C and FIG. 42F), radial displacement (FIG. 42D and FIG. 42H) for both groups are presented, which shows in following treatment period, BT2 therapy led to significant increased myocardial wall motion. *, p<0.05, **, p<0.01, data presented as mean±SEM.

FIGS. 43A-43F show BCKDK inhibitory therapy effects on diastolic LV mechanics. Mice were subjected to sham and TAC procedures and followed for a 2 wk-period to provoke cardiac dysfunction. Then, animals were randomized to treatment with BT2 (n=9) or vehicle (n=8) at week 2 post-TAC. Mean and changes of diastolic longitudinal strain rate (FIG. 43A and FIG. 43D), diastolic radial strain rate (FIG. 43B and FIG. 43E) and diastolic circumferential strain rate (FIG. 43C and FIG. 43F) for both groups are presented, which shows in following treatment period, BT2 therapy led to significant increased myocardial diastolic function. *, p<0.05, **, p<0.01, data presented as mean±SEM.

FIGS. 44A-44D show metabolic genes expressed in pressure-overloaded mouse heart. FIG. 44A shows mitochondrial DNA amount analyzed by means of quantitative PCR using primers specific for the mitochondrial cytochrome b (CytB) gene and normalized to genomic DNA by amplification of the large ribosomal protein p0 (36B4) nuclear gene. PGC-1a mRNA was analyzed by means of quantitative RT-PCR. Data presented as mean±SEM (FIG. 44B). mRNA expression of genes involving in glucose transportation (Glut1, Glut4) and oxidation (PDK4) were analyzed by quantitative PCR (FIG. 44C) and mRNA expression of genes involving in fatty acids transport (CD36), and β-oxidation (Acox1, Acadl and Acadm) were analyzed by quantitative PCR. *, p<0.05, data presented as mean±SEM (FIG. 44D).

FIGS. 45A-45E show BT2 treatment was not toxic to mouse cardiac function. Mice were subjected to sham procedure and 2 weeks later animals were randomized to treatment with BT2 (n=3) or vehicle (n=3). Mean left ventricular ejection fraction (EF) (FIG. 45A) and left ventricular internal diameter at systole (LVIDs) (FIG. 45B) and body weight for (FIG. 45C) both groups are presented which showed no significant effects during the observed period of time. After 6 weeks of administration, (FIG. 45D) Representative M-mode echocardiographs of mouse hearts following post-sham treated with vehicle or BT2 are presented. FIG. 45E shows the expression of total, phosphorylated BCKD subunit E1α in mouse heart were determined by Western blot (left). The average phosphorylated E1α level was normalized by total E1α level (right). *, p<0.05, data presented as mean±SEM.)

FIG. 46 shows a table of the KEGG pathways enriched in pressure overload-induced failing mouse hearts.

FIG. 47 shows a table of downregulated genes in pressure overload-induced failing mouse hearts.

FIG. 48 shows a table of proteins identified by upstream regulator analysis involved in the expression of downregulated genes in pressure overload-induced failing mouse hearts.

### DETAILED DESCRIPTION OF THE INVENTION

The present invention relates, in part, to a discovery that BCAA defects in cardiomyocytes are associated with heart failure and in fat tissue are associated with obesity and diabetes. Specifically, BCAA downstream metabolites, including branched-chain keto acids and 3,3-dimethylacrylic acid (DMAA) have potent signaling effects that impact cellular viability, adipogenesis, and inflammatory status of fatty tissue. In addition, BCAA catabolic flux defects can be restored by inhibiting BCKD kinase in the pathological settings of heart failure and diabetes associated with obesity.

Accordingly, the present invention provides, at least, methods of diagnosing and/or prognosing a disease or disorder in a subject with a biomarker selected from BCAA and its metabolites, such as branched-chain keto acids and 3,3-dimethylacrylic acid (DMAA). The present invention also provides an in vitro assay to analyze the levels of BCAA and/or its metabolites for such diagnosis and/or prognosis. Similarly, such biomarkers may be used to predict and/or measure the effectiveness of a therapy to a subject having such disease or disorder, wherein the therapy comprises, e.g., a supplement of BCAA and/or its metabolites and/or an inhibitor or antagonist of a natural inhibitor of BCAA (such as BCKDK). In some embodiments, a BCKDK inhibitor (such as BT-2 or related compounds) may be administered to the subject to treat the disease or disorder. Such disease or disorder may refer to any metabolic disease or disorder, such as, without limitation, cardiovascular diseases (e.g., heart failure), diabetes, overweight, etc. In some embodiments, the subject has glucose intolerance or insulin resistance. In some embodiments, the subject has BCAA and/or BCKD deficiency. In some embodiments, the subject has ischemia/reperfusion injury and/or organ damage. Accordingly, the present invention also provides a method of treating a subject having any such metabolic disturbance or disorder with BCAA, one or more BCAA metabolites, BCKA, and/or BCKDK inhibitors. Optionally, such method may further comprise an additional therapy, such as a high-BCAA diet.

### I. Definitions

The articles “a” and “an” are used herein to refer to one or to more than one (i.e. to at least one) of the grammatical object of the article. By way of example, “an element” means one element or more than one element.

The term “administering” is intended to include routes of administration which allow an agent (such as the compositions described herein) to perform its intended function. Examples of routes of administration for treatment of a body which can be used include injection (subcutaneous, intravenous, parenterally, intraperitoneally, intrathecal, etc.), oral, inhalation, and transdermal routes. The injection can be bolus injections or can be continuous infusion. Depending on the route of administration, the agent can be coated with or disposed in a selected material to protect it from natural conditions which may detrimentally affect its ability to perform its intended function. The agent may be administered alone, or in conjunction with a pharmaceutically acceptable carrier. The agent also may be administered as a prodrug, which is converted to its active form in vivo. In some embodiments, the agent is orally administered. In other embodiments, the agent is administered through anal and/or colorectal route.

The term “increased/decreased amount” or “increased/decreased level” refers to increased or decreased absolute and/or relative amount and/or value of a biomarker (e.g., BCAA and/or its metabolites, as described herein) in a subject, as compared to the amount and/or value of the same biomarker in the same subject in a prior time and/or in a normal and/or control subject.

The amount of a biomarker (e.g., BCAA and/or its metabolites, as described herein) in a subject is “significantly” higher or lower than the normal amount of the biomarker, if the amount of the biomarker is greater or less, respectively, than the normal level by an amount greater than the standard error of the assay employed to assess amount, and preferably at least 20%, 30%, 40%, 50%, 60%, 70%, 80%, 90%, 100%, 150%, 200%, 300%, 350%, 400%, 500%, 600%, 700%, 800%, 900%, 1000% or than that amount. Alternately, the amount of the biomarker in the subject can be considered “significantly” higher or lower than the normal amount if the amount is at least about two, and preferably at least about three, four, or five times, higher or lower, respectively, than the normal amount of the biomarker. Such “significance” can also be applied to any other measured parameter described herein, such as for expression, inhibition, cytotoxicity, cell growth, and the like.

The term “assigned score” refers to the numerical value designated for each of the biomarkers after being measured in a patient sample. The assigned score correlates to the absence, presence or inferred amount of the biomarker in the sample. The assigned score can be generated manually (e.g., by visual inspection) or with the aid of instrumentation for image acquisition and analysis. In certain embodiments, the assigned score is determined by a qualitative assessment, for example, detection of a fluorescent readout on a graded scale, or quantitative assessment. In certain embodiments, an “aggregate score,” which refers to the combination of assigned scores from a plurality of measured biomarkers, is determined. For example, the aggregate score may be a summation of assigned scores. Alternatively, combination of assigned scores may involve performing mathematical operations on the assigned scores before combining them into an aggregate score. In certain embodiments, the aggregate score is also referred to herein as the “predictive score.”

The term “biomarker” refers to a measurable parameter of the present invention that has been determined to be predictive of the effects of the therapy described herein, either alone or in combination with at least one other therapies, on a target disease or disorder described herein. Biomarkers can include, without limitation, BCAA and its metabolites, amino acid metabolites, and metabolic index parameters of a subject, including those shown in the Examples, the Figures, and otherwise described herein. As described herein, a biomarker may be detected and analyzed by any methods, such as detecting and/or quantifying the BCAA and its metabolites by in vivo or in vitro assays, tec. A metabolite biomarker (e.g., branched-chain keto acids and 3.3-dimethylacrylic acid (DMAA)) may be detected and/or quantified by any methods for chemicals. A metabolic biomarker (e.g., body mass index (BMI), Yale Food Addiction Scale (YFAS), hunger (fasting), desire for high calorie food, etc.) may be measured by any suitable methods.

The term “antibody” as used herein also includes an “antigen-binding portion” of an antibody (or simply “antibody portion”). The term “antigen-binding portion”, as used herein, refers to one or more fragments of an antibody that retain the ability to specifically bind to an antigen (e.g., a biomarker (such as BCAA and its metabolites) polypeptide or fragment thereof, or a target for treatment (such as BCKDK)). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al. (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent polypeptides (known as single chain Fv (scFv); see e.g., Bird et al. (1988) Science 242:423-426; and Huston et al. (1988) Proc. Natl. Acad. Sci. USA 85:5879-5883; and Osbourn et al. 1998, Nature Biotechnology 16: 778). Such single chain antibodies are also intended to be encompassed within the term “antigen-binding portion” of an antibody. Any VH and VL sequences of specific scFv can be linked to human immunoglobulin constant region cDNA or genomic sequences, in order to generate expression vectors encoding complete IgG polypeptides or other isotypes. VH and VL can also be used in the generation of Fab, Fv or other fragments of immunoglobulins using either protein chemistry or recombinant DNA technology. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123).

Still further, an antibody or antigen-binding portion thereof may be part of larger immunoadhesion polypeptides, formed by covalent or noncovalent association of the antibody or antibody portion with one or more other proteins or peptides. Examples of such immunoadhesion polypeptides include use of the streptavidin core region to make a tetrameric scFv polypeptide (Kipriyanov, S. M., et al. (1995) Human Antibodies and Hybridomas 6:93-101) and use of a cysteine residue, biomarker peptide and a C-terminal polyhistidine tag to make bivalent and biotinylated scFv polypeptides (Kipriyanov, S. M., et al. (1994) Mol. Immunol. 31:1047-1058). Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion polypeptides can be obtained using standard recombinant DNA techniques, as described herein.

The term “body fluid” refers to fluids that are excreted or secreted from the body as well as fluids that are normally not (e.g., amniotic fluid, aqueous humor, bile, blood and blood plasma, cerebrospinal fluid, cerumen and earwax, cowper's fluid or pre-ejaculatory fluid, chyle, chyme, stool, female ejaculate, interstitial fluid, intracellular fluid, lymph, menses, breast milk, mucus, pleural fluid, pus, saliva, sebum, semen, serum, sweat, synovial fluid, tears, urine, vaginal lubrication, vitreous humor, vomit). For example, any body fluid may be taken to detect and/or measure at least one biomarker described herein.

The term “control” refers to any reference standard suitable to provide a comparison to the expression products in the test sample. In certain embodiments, the control comprises obtaining a “control sample” from which expression product levels are detected and compared to the expression product levels from the test sample. Such a control sample may comprise any suitable sample, including but not limited to a sample from a control subject (can be stored sample or previous sample measurement) with a known outcome; normal tissue or cells isolated from a subject, such as a normal subject or the subject with obesity or other metabolic disturbance or intolerance, cultured primary cells/tissues isolated from a subject such as a normal subject or the subject with such disease or disorder, adjacent normal cells/tissues obtained from the same organ or body location of the normal subject or the subject, a tissue or cell sample isolated from a normal subject, or a primary cells/tissues obtained from a depository. In other preferred embodiments, the control may comprise a reference standard expression product level from any suitable source, including but not limited to housekeeping genes, an expression product level range from normal tissue (or other previously analyzed control sample), a previously determined expression product level range within a test sample from a group of patients, or a set of patients with a certain outcome (for example, survival for one, two, three, four years, etc.) or receiving a certain treatment. It will be understood by those of skill in the art that such control samples and reference standard expression product levels can be used in combination as controls in the methods of the present invention. In the former case, the specific expression product level of each patient can be assigned to a percentile level of expression, or expressed as either higher or lower than the mean or average of the reference standard expression level. In other preferred embodiments, the control may comprise normal cells, cells from patients treated with combination chemotherapy. In other embodiments, the control may also comprise a measured value for example, average level of expression of a particular gene in a population compared to the level of expression of a housekeeping gene in the same population.

A “kit” is any manufacture (e.g., a package or container) comprising at least one reagent, e.g. a probe or small molecule, for specifically detecting and/or affecting the expression of a marker of the present invention. The kit may be promoted, distributed, or sold as a unit for performing the methods of the present invention. The kit may comprise one or more reagents necessary to express a composition useful in the methods of the present invention. In certain embodiments, the kit may further comprise a reference standard. One skilled in the art can envision many such controls, including, but not limited to, common molecules. Reagents in the kit may be provided in individual containers or as mixtures of two or more reagents in a single container. In addition, instructional materials which describe the use of the compositions within the kit can be included.

The term “neoadjuvant therapy” refers to a treatment given before the primary treatment.

The “normal” level of expression and/or activity of a biomarker is the level of expression and/or activity of the biomarker in cells of a subject, e.g., a human patient, not afflicted with the disease or disorder described herein. An “over-expression” or “significantly higher level of expression” of a biomarker refers to an expression level in a test sample that is greater than the standard error of the assay employed to assess expression, and is preferably at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more higher than the expression activity or level of the biomarker in a control sample (e.g., sample from a healthy subject not having the biomarker associated disease or disorder) and preferably, the average expression level of the biomarker in several control samples. A “significantly lower level of expression” of a biomarker refers to an expression level in a test sample that is at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more lower than the expression level of the biomarker in a control sample (e.g., sample from a healthy subject not having the biomarker associated disease) and preferably, the average expression level of the biomarker in several control samples. The same determination can be made to determine overactivity or underactivity.

In some embodiments, the instant invention is drawn to a therapeutic composition for treating a disease or disorder described herein in a subject with a supplement of BCAA, its metabolites, BCKA, or a BCKDK inhibitor (e.g., BT-2). Such disease or disorder may include, e.g., metabolic disturbance or intolerance, such as glucose intolerance or insulin resistance, cardiovascular diseases (e.g., heart failure), obesity, overweight, cancer (such as pancreatic cancer), autism, etc.

In some embodiments, the weight loss may be measured by BMI change in 6 months, hunger (fasting), Yale Food Addiction Scale (YFAS), excess weight loss (% EWL), and/or desire for high caloric food.

In some embodiments, the therapeutic composition described herein further comprises another agent capable of treating the disease or disorder described herein.

In some embodiments, the subject is not obese. In other embodiments, the subject described herein has obesity. The term “obesity” can refer to any condition in which the subject is overweight relative to a control subject or the same subject at a prior time. Obesity is generally defined by measuring the body mass index (BMI), defined as the body mass divided by the square of the body height, and is universally expressed in units of kg/m², resulting from mass in kilograms and height in meters. Excess body weight (EBW) is defined as the amount of weight that is in excess of the ideal body weight (IBW). Ideal body weight is conventionally determined by the Metropolitan Life Tables, or as a BMI of 25 kg/m². In 1991, the National Institutes of Health defined morbid obesity as a BMI of ≥35 kg/m² and severe, obesity-related comorbidity as a BMI of ≥40 kg/m². Generally, a BMI of about 25.0-29.9 is referred to as overweight. A BMI value of about 30-34.9 is referred to obesity (class 1). A BMI value of about 35-39.9 is referred to severe obesity (class 2). A BMI value of about 40-49.9 is referred to severe obesity (class 3). A BMI value above about 50 is referred to superobesity. The term “obesity” preferably refers to a status of at least being overweight, for example, when the BMI value of a subject is at least about 25.0, or above.

In some embodiments, the subject described herein has a cardiovascular disease (such as heart failure). The term “cardiovascular disease” refers to a class of diseases that involve the heart and/or blood vessels. Cardiovascular disease includes coronary artery diseases (CAD) such as angina and myocardial infarction (commonly known as a heart attack). Other CVDs are stroke, heart failure, hypertensive heart disease, rheumatic heart disease, cardiomyopathy, heart arrhythmia, congenital heart disease, valvular heart disease, carditis, aortic aneurysms, peripheral artery disease, and venous thrombosis. The term “heart failure,” often referred to as congestive heart failure (CHF), occurs when the heart is unable to pump sufficiently to maintain blood flow to meet the body's needs. Signs and symptoms commonly include shortness of breath, excessive tiredness, and leg swelling. The condition is diagnosed based on the history of the symptoms and a physical examination with confirmation by echocardiography. Blood tests, electrocardiography, and chest radiography may be useful to determine the underlying cause (Chronic Heart Failure: National Clinical Guideline for Diagnosis and Management in Primary and Secondary Care: Partial Update. National Clinical Guideline Centre: 34-47, August 2012). Treatment depends on the severity and cause of the disease. In people with chronic stable mild heart failure, treatment commonly consists of lifestyle modifications such as stopping smoking, physical exercise, and dietary changes, as well as medications. In those with heart failure due to left ventricular dysfunction, angiotensin converting enzyme inhibitors or angiotensin receptor blockers along with beta blockers are recommended. For those with severe disease, aldosterone antagonists, or hydralazine with a nitrate may be used. Diuretics are useful for preventing fluid retention. Sometimes, depending on the cause, an implanted device such as a pacemaker or an implantable cardiac defibrillator may be recommended. In some moderate or severe cases cardiac resynchronization therapy (CRT) may be suggested or cardiac contractility modulation may be of benefit. A ventricular assist device or occasionally a heart transplant may be recommended in those with severe disease despite all other measures.

The therapeutic composition described herein may be administered, alone or in combination with a therapeutically acceptable carrier, to the subject through any suitable route. Such administration may be systemic (e.g., IV) or local (e.g., directly to stomach or intestines). A preferred administration route is oral administration. Other routes (e.g., rectal) may be also used.

The term “pre-determined” biomarker amount and/or activity measurement(s) may be a biomarker amount and/or activity measurement(s) used to, by way of example only, evaluate a subject that may be selected for a particular treatment, evaluate a response to a treatment such as using a composition described herein, alone or in combination with other therapy to improve weight loss. A pre-determined biomarker amount and/or activity measurement(s) may be determined in populations of patients with or without a disease (e.g., obesity, overweight, heart failure, etc.). The pre-determined biomarker amount and/or activity measurement(s) can be a single number, equally applicable to every patient, or the pre-determined biomarker amount and/or activity measurement(s) can vary to reflect differences among specific subpopulations of patients. Age, weight, height, and other factors of a subject may affect the pre-determined biomarker amount and/or activity measurement(s) of the individual. Furthermore, the pre-determined biomarker amount and/or activity can be determined for each subject individually. In certain embodiments, the amounts determined and/or compared in a method described herein are based on absolute measurements. In other embodiments, the amounts determined and/or compared in a method described herein are based on relative measurements, such as ratios (e.g., serum biomarker normalized to the expression of housekeeping or otherwise generally constant biomarker). The pre-determined biomarker amount and/or activity measurement(s) can be any suitable standard. For example, the pre-determined biomarker amount and/or activity measurement(s) can be obtained from the same or a different subject for whom a subject selection is being assessed. In some embodiments, the pre-determined biomarker amount and/or activity measurement(s) can be obtained from a previous assessment of the same subject. In such a manner, the progress of the selection of the patient can be monitored over time. In addition, the control can be obtained from an assessment of another subject or multiple subjects, e.g., selected groups of subjects. In such a manner, the extent of the selection of the subject for whom selection is being assessed can be compared to suitable other subjects, e.g., other subjects who are in a similar situation to the human of interest, such as those suffering from similar or the same condition(s) and/or of the same ethnic group.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The term “prognosis” includes a prediction of the probable course and outcome of obesity or the likelihood of recovery from the disease or disorder described herein. In some embodiments, the use of statistical algorithms provides a prognosis of such disease or disorder in an individual.

The term “sample” used for detecting or determining the presence or level of at least one biomarker is typically brain tissue, cerebrospinal fluid, whole blood, plasma, serum, saliva, urine, stool (e.g., feces), tears, and any other bodily fluid (e.g., as described above under the definition of “body fluids”), or a tissue sample (e.g., biopsy) such as a small intestine, colon sample, or surgical resection tissue. In certain instances, the method of the present invention further comprises obtaining the sample from the individual prior to detecting or determining the presence or level of at least one marker in the sample.

The term “synergistic effect” refers to the combined effect of two or more agents described herein can be greater than the sum of the separate effects of any one of agents alone.

The term “survival” includes all of the following: survival until mortality, also known as overall survival (wherein said mortality may be either irrespective of cause or obesity related); “recurrence-free survival” (wherein the weight loss fails and obesity re-occurs); obesity-free survival. The length of said survival may be calculated by reference to a defined start point (e.g. time of diagnosis or start of treatment) and end point (e.g., death or recurrence). In addition, criteria for efficacy of treatment can be expanded to include response to other therapies within a given time period, and probability of obesity recurrence.

The term “therapeutic effect” refers to a local or systemic effect in animals, particularly mammals, and more particularly humans, caused by a pharmacologically active substance. The term thus means any substance intended for use in the diagnosis, cure, mitigation, treatment or prevention of disease or in the enhancement of desirable physical or mental development and conditions in an animal or human. The phrase “therapeutically-effective amount” means that amount of such a substance that produces some desired local or systemic effect at a reasonable benefit/risk ratio applicable to any treatment. In certain embodiments, a therapeutically effective amount of a compound will depend on its therapeutic index, solubility, and the like. For example, certain compounds discovered by the methods of the present invention may be administered in a sufficient amount to produce a reasonable benefit/risk ratio applicable to such treatment.

### II. Subjects

In certain embodiments, the subject suitable for the compositions and methods disclosed herein is a mammal (e.g., mouse, rat, primate, non-human mammal, domestic animal, such as a dog, cat, cow, horse, and the like), and is preferably a human. In other embodiments, the subject is an animal model of metabolic disturbance or intolerance.

In other embodiments of the methods of the present invention, the subject has not undergone treatment for the disease or disorder. In still other embodiments, the subject has undergone treatment for the disease or disorder.

The methods of the present invention can be used to treat and/or determine the responsiveness to a composition described herein, alone or in combination with other therapies to achieve weight loss, in subjects such as those described herein.

### 3. Pharmaceutical Compositions

The present invention provides pharmaceutically acceptable compositions of the compositions disclosed herein. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles.

The therapeutic compositions of the present invention may also include known antioxidants, buffering agents, and other agents such as coloring agents, flavorings, vitamins or

minerals.

In some embodiments, the therapeutic compositions of the present invention are combined with a carrier which is physiologically compatible with the tissue of the species to which it is administered. Carriers can be comprised of solid-based, dry materials for formulation into tablet, capsule or powdered form; or the carrier can be comprised of liquid or gel-based materials for formulations into liquid or gel forms. The specific type of carrier, as well as the final formulation depends, in part, upon the selected route(s) of administration. The therapeutic composition of the present invention may also include a variety of carriers and/or binders. A preferred carrier is micro-crystalline cellulose (MCC) added in an amount sufficient to complete the one gram dosage total weight. Carriers can be solid-based dry materials for formulations in tablet, capsule or powdered form, and can be liquid or gel-based materials for formulations in liquid or gel forms, which forms depend, in part, upon the routes of administration. Typical carriers for dry formulations include, but are not limited to: trehalose, malto-dextrin, rice flour, microcrystalline cellulose (MCC) magnesium stearate, inositol, FOS, GOS, dextrose, sucrose, and like carriers. Suitable liquid or gel-based carriers include but are not

limited to: water and physiological salt solutions; urea; alcohols and derivatives (e.g., methanol, ethanol, propanol, butanol); glycols (e.g., ethylene glycol, propylene glycol, and the like). Preferably, water-based carriers possess a neutral pH value (i.e., pH 7.0). Other carriers or agents for administering the compositions described herein are known in the art, e.g., in U.S. Pat. No. 6,461,607.

The phrase “pharmaceutically acceptable” is employed herein to refer to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of one or more agents as disclosed herein.

The present invention also encompasses kits for detecting and/or modulating biomarkers described herein. A kit of the present invention may also include instructional materials disclosing or describing the use of the kit or an antibody of the disclosed invention in a method of the disclosed invention as provided herein. A kit may also include additional components to facilitate the particular application for which the kit is designed. For example, a kit may additionally contain means of detecting the label (e.g., enzyme substrates for enzymatic labels, filter sets to detect fluorescent labels, appropriate secondary labels such as a sheep anti-mouse-HRP, etc.) and reagents necessary for controls (e.g., control biological samples or standards). A kit may additionally include buffers and other reagents recognized for use in a method of the disclosed invention. Non-limiting examples include agents to reduce non-specific binding, such as a carrier protein or a detergent.

### Further Uses and Methods of the Present Invention

The compositions described herein can be used in a variety of diagnostic, prognostic, and therapeutic applications. In any method described herein, such as a diagnostic method, prognostic method, therapeutic method, or combination thereof, all steps of the method can be performed by a single actor or, alternatively, by more than one actor. For example, diagnosis can be performed directly by the actor providing therapeutic treatment. Alternatively, a person providing a therapeutic agent can request that a diagnostic assay be performed. The diagnostician and/or the therapeutic interventionist can interpret the diagnostic assay results to determine a therapeutic strategy. Similarly, such alternative processes can apply to other assays, such as prognostic assays.

### 1) Predictive Medicine

The present invention can pertain to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining the amount and/or activity level of a biomarker described herein (e.g., obesity, overweight, heart failure, or other metabolic disturbance or intolerance, such as glucose and/or insulin intolerance) in the context of a biological sample (e.g., blood, serum, cells, or tissue) to thereby determine whether an individual afflicted with the disease or disorder is likely to respond to a composition as disclosed herein. Such assays can be used for prognostic or predictive purpose alone, or can be coupled with a therapeutic intervention to thereby prophylactically treat an individual prior to the onset or after recurrence of a disorder characterized by or associated with biomarker polypeptide, nucleic acid expression or activity. The skilled artisan will appreciate that any method can use one or more (e.g., combinations) of biomarkers described herein, such as those in the tables, figures, examples, and otherwise described in the specification.

### 2) Diagnostic Assays

The present invention provides, in part, methods, systems, and code for accurately classifying whether a biological sample is associated with the disease or disorder described herein that is likely to respond to a composition as disclosed herein. In some embodiments, the present invention is useful for classifying a sample (e.g., from a subject) as associated with or at risk for responding to or not responding to a composition as disclosed herein using a statistical algorithm and/or empirical data (e.g., the amount or activity of a biomarker described herein, such as in the tables, figures, examples, and otherwise described in the specification).

An exemplary method for detecting the amount or activity of a biomarker described herein, and thus useful for classifying whether a sample is likely or unlikely to respond to a composition as disclosed herein involves obtaining a biological sample from a test subject and contacting the biological sample with an agent, such as a protein-binding agent like an antibody or antigen-binding fragment thereof, or a nucleic acid-binding agent like an oligonucleotide, capable of detecting the amount or activity of the biomarker in the biological sample. In some embodiments, at least one antibody or antigen-binding fragment thereof is used, wherein two, three, four, five, six, seven, eight, nine, ten, or more such antibodies or antibody fragments can be used in combination (e.g., in sandwich ELISAs) or in serial. In certain instances, the statistical algorithm is a single learning statistical classifier system. For example, a single learning statistical classifier system can be used to classify a sample as a based upon a prediction or probability value and the presence or level of the biomarker. The use of a single learning statistical classifier system typically classifies the sample as, for example, a likely therapy responder or progressor sample with a sensitivity, specificity, positive predictive value, negative predictive value, and/or overall accuracy of at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

Other suitable statistical algorithms are well-known to those of skill in the art. For example, learning statistical classifier systems include a machine learning algorithmic technique capable of adapting to complex data sets (e.g., panel of markers of interest) and making decisions based upon such data sets. In some embodiments, a single learning statistical classifier system such as a classification tree (e.g., random forest) is used. In other embodiments, a combination of 2, 3, 4, 5, 6, 7, 8, 9, 10, or more learning statistical classifier systems are used, preferably in tandem. Examples of learning statistical classifier systems include, but are not limited to, those using inductive learning (e.g., decision/classification trees such as random forests, classification and regression trees (C&RT), boosted trees, etc.), Probably Approximately Correct (PAC) learning, connectionist learning (e.g., neural networks (NN), artificial neural networks (ANN), neuro fuzzy networks (NFN), network structures, perceptrons such as multi-layer perceptrons, multi-layer feed-forward networks, applications of neural networks, Bayesian learning in belief networks, etc.), reinforcement learning (e.g., passive learning in a known environment such as naive learning, adaptive dynamic learning, and temporal difference learning, passive learning in an unknown environment, active learning in an unknown environment, learning action-value functions, applications of reinforcement learning, etc.), and genetic algorithms and evolutionary programming. Other learning statistical classifier systems include support vector machines (e.g., Kernel methods), multivariate adaptive regression splines (MARS), Levenberg-Marquardt algorithms, Gauss-Newton algorithms, mixtures of Gaussians, gradient descent algorithms, and learning vector quantization (LVQ). In certain embodiments, the method of the present invention further comprises sending the sample classification results to a clinician, e.g., an oncologist.

In other embodiments, the diagnosis of a subject is followed by administering to the individual a therapeutically effective amount of a defined treatment based upon the diagnosis.

In some embodiments, the methods further involve obtaining a control biological sample (e.g., biological sample from a subject who does not have obesity), a biological sample from the subject during remission, or a biological sample from the subject during treatment for developing obesity progressing despite a composition as disclosed herein.

### 3) Prognostic Assays

The diagnostic methods described herein can furthermore be utilized to identify subjects having or at risk of developing obesity or weight gain that is likely or unlikely to be responsive to a composition as disclosed herein. The assays described herein, such as the preceding diagnostic assays or the following assays, can be utilized to identify a subject having or at risk of developing a disorder associated with a misregulation of the amount or activity of at least one biomarker described herein. Alternatively, the prognostic assays can be utilized to identify a subject having or at risk for developing a disorder associated with a misregulation of the at least one biomarker described herein. Furthermore, the prognostic assays described herein can be used to determine whether a subject can be administered a composition as disclosed herein and/or an additional therapeutic regimen to treat a disease or disorder associated with the aberrant biomarker expression or activity.

An “isolated” or “purified” biomarker (e.g., metabolic products) is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, 20%, 10%, or 5% (by dry weight) of heterologous protein (also referred to herein as a “contaminating protein”). When the protein or biologically active portion thereof is recombinantly produced, it is also preferably substantially free of culture medium, i.e., culture medium represents less than about 20%, 10%, or 5% of the volume of the protein preparation. When the protein is produced by chemical synthesis, it is preferably substantially free of chemical precursors or other chemicals, i.e., it is separated from chemical precursors or other chemicals which are involved in the synthesis of the protein. Accordingly such preparations of the protein have less than about 30%, 20%, 10%, 5% (by dry weight) of chemical precursors or compounds other than the polypeptide of interest.

In some embodiments, agents that specifically bind to a biomarker protein other than antibodies are used, such as peptides. Peptides that specifically bind to a biomarker protein can be identified by any means known in the art. For example, specific peptide binders of a biomarker protein can be screened for using peptide phage display libraries.

### Sampling Methods

In some embodiments, biomarker amount and/or activity measurement(s) in a sample from a subject is compared to a predetermined control (standard) sample. The control sample can be from the same subject or from a different subject. The control sample is typically a normal, non-diseased sample. However, in some embodiments, such as for staging of disease or for evaluating the efficacy of treatment, the control sample can be from a diseased tissue. The control sample can be a combination of samples from several different subjects. In some embodiments, the biomarker amount and/or activity measurement(s) from a subject is compared to a pre-determined level. This pre-determined level is typically obtained from normal samples. As described herein, a “pre-determined” biomarker amount and/or activity measurement(s) may be a biomarker amount/levels, and/or activity measurement(s) used to, by way of example only, evaluate a subject that may be selected for treatment (e.g., based on the number of genomic mutations and/or the number of genomic mutations causing non-functional proteins for DNA repair genes), evaluate a response to a composition as disclosed herein, alone or in combination with other NK immunotherapies and with one or more additional anti-obesity or weight loss therapies. A pre-determined biomarker amount/levels and/or activity measurement(s) may be determined in populations of patients with or without obesity. The pre-determined biomarker amount and/or activity measurement(s) can be a single number, equally applicable to every patient, or the pre-determined biomarker amount and/or activity measurement(s) can vary according to specific subpopulations of patients. Age, weight, height, and other factors of a subject may affect the pre-determined biomarker amount and/or activity measurement(s) of the individual. Furthermore, the pre-determined biomarker amount and/or activity can be determined for each subject individually. In some embodiments, the amounts determined and/or compared in a method described herein are based on absolute measurements.

The term “disease” or “disorder” includes a metabolic disorder (such as a metabolic disturbance or intolerance) in the subject. For example, obesity, overweight, heart failure, glucose or insulin intolerance are all included in the scope of “diseases” or “disorder” described herein, whether or not it fits in the medical definition of a disease according to a medical professional. A metabolic disorder includes any disease, disorder, or symptom when normal metabolic process in body is disturbed, due to either inherited or acquired causes. Some of the possible symptoms that can occur with metabolic disorders are: lethargy, weight loss, jaundice, seizures, cancer (such as pancreatic cancer), autism, etc. Metabolic syndrome includes, at least, abdominal (central) obesity (cf. TOFI), elevated blood pressure, elevated fasting plasma glucose, high serum triglycerides, low high-density lipoprotein (HDL) levels, etc. Common metabolic disorders include, at least, obesity, diabetes (e.g., type II), impaired glucose tolerance, impaired fasting glucose or insulin resistance, dyslipidemia, microalbuminuria, and hypertension.

In other embodiments, the amounts determined and/or compared in a method described herein are based on relative measurements, such as ratios (e.g., biomarker amount, levels, and/or activity before a treatment vs. after a treatment, such biomarker measurements relative to a spiked or man-made control, such biomarker measurements relative to the expression of a housekeeping gene, and the like). For example, the relative analysis can be based on the ratio of pre-treatment biomarker measurement as compared to post-treatment biomarker measurement. Pre-treatment biomarker measurement can be made at any time prior to initiation of anti-obesity or weight loss therapy. Post-treatment biomarker measurement can be made at any time after initiation of therapy. In some embodiments, post-treatment biomarker measurements are made 1, 2, 3, 4, 5, 6, 7, 8, 9, 10, 11, 12, 13, 14, 15, 16, 17, 18, 19, 20 weeks or more after initiation of therapy, and even longer toward indefinitely for continued monitoring. Treatment can comprise weight loss therapy, such as a therapeutic regimen comprising a composition as disclosed herein, or further in combination with other agents.

The pre-determined biomarker amount and/or activity measurement(s) can be any suitable standard. For example, the pre-determined biomarker amount and/or activity measurement(s) can be obtained from the same or a different human for whom a patient selection is being assessed. In some embodiments, the pre-determined biomarker amount and/or activity measurement(s) can be obtained from a previous assessment of the same patient. In such a manner, the progress of the selection of the patient can be monitored over time. In addition, the control can be obtained from an assessment of another human or multiple humans, e.g., selected groups of humans, if the subject is a human. In such a manner, the extent of the selection of the human for whom selection is being assessed can be compared to suitable other humans, e.g., other humans who are in a similar situation to the human of interest, such as those suffering from similar or the same condition(s) and/or of the same ethnic group.

In some embodiments of the present invention the change of biomarker amount/levels and/or activity measurement(s) from the pre-determined level is about 0.1, 0.2, 0.3, 0.4, 0.5, 0.6, 0.7, 0.8, 0.9, 1.0, 1.5, 2.0, 2.5, 3.0, 3.5, 4.0, 4.5, or 5.0 fold or greater, or any range in between, inclusive. Such cutoff values apply equally when the measurement is based on relative changes, such as based on the ratio of pre-treatment biomarker measurement as compared to post-treatment biomarker measurement.

Biological samples can be collected from a variety of sources from a patient including a body fluid sample, cell sample, or a tissue sample comprising nucleic acids and/or proteins. “Body fluids” refer to fluids that are excreted or secreted from the body as well as fluids that are normally not (e.g., amniotic fluid, aqueous humor, bile, blood and blood plasma, cerebrospinal fluid, cerumen and earwax, cowper's fluid or pre-ejaculatory fluid, chyle, chyme, stool, female ejaculate, interstitial fluid, intracellular fluid, lymph, menses, breast milk, mucus, pleural fluid, pus, saliva, sebum, semen, serum, sweat, synovial fluid, tears, urine, vaginal lubrication, vitreous humor, vomit). In preferred embodiments, the subject and/or control sample is selected from the group consisting of cells, cell lines, histological slides, paraffin embedded tissues, biopsies, whole blood, nipple aspirate, serum, plasma, buccal scrape, saliva, cerebrospinal fluid, urine, stool, and bone marrow. In some embodiments, the sample is serum, plasma, or urine. In other embodiments, the sample is serum.

The samples can be collected from individuals repeatedly over a longitudinal period of time (e.g., once or more on the order of days, weeks, months, annually, biannually, etc.). Obtaining numerous samples from an individual over a period of time can be used to verify results from earlier detections and/or to identify an alteration in biological pattern as a result of, for example, disease progression, drug treatment, etc. For example, subject samples can be taken and monitored every month, every two months, or combinations of one, two, or three month intervals according to the present invention. In addition, the biomarker amount and/or activity measurements of the subject obtained over time can be conveniently compared with each other, as well as with those of normal controls during the monitoring period, thereby providing the subject's own values, as an internal, or personal, control for long-term monitoring.

Sample preparation and separation can involve any of the procedures, depending on the type of sample collected and/or analysis of biomarker measurement(s). Such procedures include, by way of example only, concentration, dilution, adjustment of pH, removal of high abundance polypeptides (e.g., albumin, gamma globulin, and transferrin, etc.), addition of preservatives and calibrants, addition of protease inhibitors, addition of denaturants, desalting of samples, concentration of sample proteins, extraction and purification of lipids.

The sample preparation can also isolate molecules that are bound in non-covalent complexes to other protein (e.g., carrier proteins). This process may isolate those molecules bound to a specific carrier protein (e.g., albumin), or use a more general process, such as the release of bound molecules from all carrier proteins via protein denaturation, for example using an acid, followed by removal of the carrier proteins.

Removal of undesired proteins (e.g., high abundance, uninformative, or undetectable proteins) from a sample can be achieved using high affinity reagents, high molecular weight filters, ultracentrifugation and/or electrodialysis. High affinity reagents include antibodies or other reagents (e.g., aptamers) that selectively bind to high abundance proteins. Sample preparation could also include ion exchange chromatography, metal ion affinity chromatography, gel filtration, hydrophobic chromatography, chromatofocusing, adsorption chromatography, isoelectric focusing and related techniques. Molecular weight filters include membranes that separate molecules on the basis of size and molecular weight. Such filters may further employ reverse osmosis, nanofiltration, ultrafiltration and microfiltration.

Ultracentrifugation is a method for removing undesired polypeptides from a sample. Ultracentrifugation is the centrifugation of a sample at about 15,000-60,000 rpm while monitoring with an optical system the sedimentation (or lack thereof) of particles. Electrodialysis is a procedure which uses an electromembrane or semipermable membrane in a process in which ions are transported through semi-permeable membranes from one solution to another under the influence of a potential gradient. Since the membranes used in electrodialysis may have the ability to selectively transport ions having positive or negative charge, reject ions of the opposite charge, or to allow species to migrate through a semipermable membrane based on size and charge, it renders electrodialysis useful for concentration, removal, or separation of electrolytes.

Separation and purification in the present invention may include any procedure known in the art, such as capillary electrophoresis (e.g., in capillary or on-chip) or chromatography (e.g., in capillary, column or on a chip). Electrophoresis is a method which can be used to separate ionic molecules under the influence of an electric field. Electrophoresis can be conducted in a gel, capillary, or in a microchannel on a chip. Examples of gels used for electrophoresis include starch, acrylamide, polyethylene oxides, agarose, or combinations thereof. A gel can be modified by its cross-linking, addition of detergents, or denaturants, immobilization of enzymes or antibodies (affinity electrophoresis) or substrates (zymography) and incorporation of a pH gradient. Examples of capillaries used for electrophoresis include capillaries that interface with an electrospray.

Capillary electrophoresis (CE) is preferred for separating complex hydrophilic molecules and highly charged solutes. CE technology can also be implemented on microfluidic chips. Depending on the types of capillary and buffers used, CE can be further segmented into separation techniques such as capillary zone electrophoresis (CZE), capillary isoelectric focusing (CIEF), capillary isotachophoresis (cITP) and capillary electrochromatography (CEC). CE techniques can be coupled to electrospray ionization through the use of volatile solutions, for example, aqueous mixtures containing a volatile acid and/or base and an organic such as an alcohol or acetonitrile.

Capillary isotachophoresis (cITP) is a technique in which the analytes move through the capillary at a constant speed but are nevertheless separated by their respective mobilities. Capillary zone electrophoresis (CZE), also known as free-solution CE (FSCE), is based on differences in the electrophoretic mobility of the species, determined by the charge on the molecule, and the frictional resistance the molecule encounters during migration which is often directly proportional to the size of the molecule. Capillary isoelectric focusing (CIEF) allows weakly-ionizable amphoteric molecules, to be separated by electrophoresis in a pH gradient. CEC is a hybrid technique between traditional high performance liquid chromatography (HPLC) and CE.

Separation and purification techniques used in the present invention include any chromatography procedures known in the art. Chromatography can be based on the differential adsorption and elution of certain analytes or partitioning of analytes between mobile and stationary phases. Different examples of chromatography include, but not limited to, liquid chromatography (LC), gas chromatography (GC), high performance liquid chromatography (HPLC), etc.

### EXAMPLES

### Example 1: Exemplary Materials and Methods Used in Example 2

Animals and Human Cohorts

PP2Cm germ-line knockout (PP2Cm-KO) mice were generated as previously described (Lu et al. (2009), supra). PP2Cm KO mice were backcrossed for more than 8 generations into a C57BL/6 background. Wild type C57BL/6 mice and PP2Cm KO mice were housed at 22° C. with a 12-hour light, 12-hour dark cycle with free access to water and standard chow. Studies were performed with male mice. Human cohorts of dilated cardiomyopathy and controls were obtained from Columbia and Duke with Institutional Review Board approval. All animal procedures were carried out in accordance with the guidelines and protocols approved by the University of California at Los Angeles Institutional Animal Care and Use Committee (IACUC). Transaortic constriction (TAC) and cardiac echocardiography were performed as reported earlier 17 on mice from different genotypes between 14 and 16 weeks of age. Compound BT2 (3,6-dichlorobenzo[b]thiophene-2-carboxylic acid) was purchased from Sigma-Aldrich and administrated by oral gavage at 40 mg·kg⁻¹·d⁻¹ as previously described (Tso et al. (2014) Journal of Biological Chemistry 289:20583-20593). Administration of BT2 started one week before TAC surgery and continued for 4 weeks post-TAC. Measurements of BCKD activity in mouse cardiac tissue and plasma BCKA/BCAA concentrations were performed as previously described (Tso et al. (2014), supra).

Transverse Aortic Constriction

In mice, transverse aortic constriction (TAC) was performed as described (Gao et al. (2016) J Clin Invest. 126:195-206) in anesthetized (pentobarbital 60 mg/kg, IP) and ventilated mice (age 14-16 weeks) to induce hypertrophy and heart failure. After left anterolateral thoracotomy with blunt dissection through the intercostal muscles, aortic constriction was induced by ligating the transverse aorta around a 27½-gauge blunt needle using 6-0 silk suture. The needle was subsequently removed. Sham-operated mice underwent a similar surgical procedure without constriction of the aorta. All mice were maintained in the same environment with regular lab chow and water ad libitum. At the end of the experiments, animals were euthanized and the hearts and lungs were removed and weighed. Hearts were dissected and tissues were either immediately immersed into 4% buffered formaldehyde or quickly frozen in liquid nitrogen for further experiments.

Echocardiography

The mice were anesthetized and maintained with 1-2% isofluorane in 95% oxygen. Echocardiography was performed with a VisualSonics Vevo 770 (VisualSonics Inc, Toronto, Canada) equipped with a 30-MHz linear transducer. A parasternal short axis view was used to obtain M-mode images for analysis of fractional shortening, ejection fraction, and other cardiac parameters.

Molecular Methods and Reagents

The details of expression vectors, transfection methods, cell culture, immunoblotting, reverse transcription-polymerase chain reaction (RT-PCR), and chromatin immunoprecipitation methods were provided below. Superoxide measurement was performed by the electron spin resonance method, and BCKA and BCAA measurements from tissue or plasma were performed following the method published by Olson et al. (2013) Anal Biochem. 439:116-122 with modifications; details are given below. The global metabolomic analysis was carried out by Metabolon, Inc. (Durham, N.C.) using heart tissues from PP2Cm-KO and wild-type male mice at 14 to 16 weeks of age. A detailed description of the analysis is given in the online-only Data Supplement.

Mitochondrial Assay

The isolation of mitochondria to measure oxygen consumption was performed as described elsewhere (Korge et al. (2011) J. Biol. Chem. 286:34851-34857). Briefly, mitochondria were isolated from heart tissue and oxygen consumption was measured using an Ocean Optics fiber optic spectrofluorometer. Mitochondria (0.25 mg/ml) were added to the assay buffer (125 mM KCl, 10 mm HEPES-KOH, pH 7.4). The oxygen concentration in the buffer was continuously recorded via an Ocean Optics FOXY fiber optic oxygen sensor. Pyruvate, malate, and glutamate were added as free acids buffered with Tris (pH 7.4) for Complex I activity assay. Addition of 0.2 mM ADP initiated oxygen consumption. NaCl or BCKA-Na mixture was added to the reaction system after the first pulse of ADP was consumed. Then the second pulse of ADP was added. The oxygen consumption rate (OCR) was calculated with each ADP addition. The relative rate of oxygen consumption was calculated by dividing the OCR of second pulse of ADP by the OCR of the first pulse of ADP. Succinate was used for Complex II activity assay in presence of rotenone (1 μM). The oxygen consumption rate (OCR) was calculated with each ADP addition. The presented data represented the average values of three independent experiments.

Western Blot Analysis

Proteins from heart tissue or cells were harvested in buffer (50 mM HEPES (pH7.4), 150 mM NaCl, 1% NP-40, 1 mM EDTA, 1 mM EGTA, 1 mM glycerophosphate, 2.5 mM sodium pyrophosphate, 1 mM Na3VO4, 20 mM NaF, 1 mM phenylmethylsulfonyl fluoride, 1 μg/mL of aprotinin, leupeptin, and pepstatin). Samples were separated on 4-12% Bis-Tris gels (Invitrogen), and transferred onto a nitrocellulose blot (Amersham). The blot was probed with the indicated primary antibodies. Protein signals were detected using HRP conjugated secondary antibodies and enhanced chemiluminescence (ECL) western blotting detection regents (Pierce). Rabbit polyclonal antisera against the E1 and E2 subunits of BCKD complex is a kind gift from Dr. Yoshiharu Shimomura (Nagoya Institute of Technology, Nagoya, JP). PP2Cm and phosphor-E1α antibodies were generated in the lab. The KLF15 primary antibody was purchased from Abcam.

Real-Time RT-PCR and Microarray Analysis

Total RNA was extracted from cells or tissues using Trizol Reagent (Invitrogen) according to the manufacturer's instructions. For neonatal mouse, 3-5 hearts were combined to extract RNA as one individual sample. Total RNA was reverse-transcribed into the first-strand cDNA using the Superscript First-Strand Synthesis Kit (Invitrogen). Then, cDNA transcripts were quantified by the Step-One Plus Real-Time PCR System (ABI) using SYBR Green (ABI). 18sRNA were used for normalization except where indicated. PCR primer information is available upon request. The cDNA from wild type and PP2Cm deficient mice was applied to Illumina MouseRef-8 v2.0 Expression BeadChips at the UCLA DNA Microarray Core Facility for whole-genome expression profiling. The data were analyzed with BeadStudio Gene Expression Module v3.4 program.

Bioinformatics Analysis

The transcriptomes of sham and failing mouse hearts 3 were analyzed using the Database for Annotation, Visualization, and Integrated Discovery (DAVID) (at the World Wide Web address of david.abcc.ncifcrf.gov). The lists of genes showing either up-regulation (782 genes) or down-regulation (653 genes) in failing hearts were separately entered into the DAVID and subjected to Functional Annotation Chart analysis with a EASE score of 0.05 using the KEGG pathway database. KEGG pathway tool was utilized through DAVID online tools to visually map down-regulated genes involved in BCAA degradation pathway in failing heart.

To predict upstream regulators of target genes, the down-regulated gene list (653 genes) was analyzed using Ingenuity Pathway Analysis (IPA) Software (at the World Wide Web address of www.ingenuity.com). A Fisher's Exact Test p-value is calculated to assess the significance of enrichment.

Expression Constructs, Cell Culture, Transient Transfection and Luciferase Assay

Mouse KLF15 cDNA was generated from heart mRNA, inserted into the pFLAG-CMV-4 expression vector, and used to generate adenovirus 4. Utilizing the NCBI GenBank, the genomic sequence of mouse PP2Cm (Ppm1k) was identified. Five proximal 5′ regions (−468, −412, −296, −254 to +20 bp relative to transcript start site, respectively) were amplified by PCR. Promoter PCR product was cloned into a firefly luciferase reporter pGL3-Basic vector (Promega, Madison, Wis.) to drive luciferase expression (PP2Cm-luc). The site-specific deletion was accomplished with the Agilent QuikChange XL site directed mutagenesis kit. Neonatal rat ventricular myocytes (NRVM) were isolated and cultured as previously described (Prosdocimo et al. (2014) J. Biol. Chem. 289:5914-5924). HeLa cell lines were maintained in DMEM (Invitrogen) supplemented with 10% fetal bovine serum, 2 mM L-glutamine, 100 U/mL penicillin, and 100 μg/ml streptomycin. Transient transfections were performed with the use of Lipofectamine 2000 reagent (Invitrogen). HeLa cells were seeded into 12 well plates at a density of 2×10⁵ cells per well the day before transfection. For each well of cells 0.2 μg of the promoter constructs were co-transfected with 0.02 m of the pSV40-Renilla vectors. The transfected cells were collected after transfection 48 hours. Luciferase activities were measured with the Dual-Luciferase Reporter Assay System (Promenade). To normalize for transfection efficiency, the promoter activity was expressed as the ratio of firefly activity to renilla activity. For each construct, more than three independent experiments were performed in triplicate.

Chromatin Immunoprecipitation (ChIP) Assay

Neonatal Rat Ventricular Myocytes (NRVM) (4.0×10⁷) were mock or FLAG-KLF15 infected with adenovirus. 48 hr post infection, cells were cross-linked with 1% formaldehyde for 10 min at room temperature. ChIP analysis was performed using SimpleChIP Enzymatic Chromatin IP Kit (Cell Signaling) according to the manufacture's protocol. Briefly, cross linking was terminated by adding glycine into cells for 5 min at room temperature. Cells were harvested by PBS/PMSF and chromatin DNA was extracted. Following Micrococcal nuclease treatment, chromatin DNA was further sheared by sonication. Immuno-precipitation was performed using Normal Rabbit IgG (Santa Cruz) or DYKDDDDK antibody (Cell Signaling) with ChIP Grade Protein G Magnetic Beads overnight. Following immune-precipitation, magnetic beads were washed with low salt buffer and high salt buffer according to the protocol, and the cross-linking was reversed by proteinase K digestion at 65 degree for 2 hr. The eluted DNA was further purified using column provided in SimpleChIP Enzymatic Chromatin IP Kit. PCR was performed to detect enrichment of rat PP2Cm promoter region. For PP2Cm, forward primer sequence is 5′-ACAAATTAAGACTAAAAAGT-3′ (SEQ ID NO:1) and reverse primer sequence is 5′-CCCACAGGAACTAGTCAAGG-3′(SEQ ID NO:2). PCR products were separated using agarose gel electrophoresis and visualized. IgG was used as negative control for ChIP specificity.

Measurement of BCAA and BCKA Concentrations in Hearts

Mice on normal chow diet were either fasted for 6 hours or overnight followed with a high protein diet (40%, Teklad) feeding for two hours before tissue collection. BCKA level in KLF15 knockout heart was measured in mice fasted for 48 hours. Human left ventricular RNA samples were obtained as previously described (Chokshi et al. (2012) Circulation 125:2844-253). In brief, myocardial specimens were collected before and after left ventricular assisted device (LVAD) implantation and explantation as a bridge to transplantation for end-stage HF patients. Control heart samples were obtained from non-failing hearts as previously described (Chokshi et al. (2012), supra). The use of all mouse and human samples was approved by the Institutional Review Board of Columbia University and Case Western Reserve University (IRB-AAAE7393). Freeze clamped mouse hearts were crushed with a metal mortar and pestle that was maintained at the temperature of liquid nitrogen. The powdered tissue was transferred to a tared tube, the weight recorded, and then processed with perchloric acid as previously described (Lynch et al. (1995) The Biochemical Journal 310 (Pt 1):197-202). A ratio of 300 μl perchloric acid per 100 mg of tissue was used. The aliquotted perchloric acid supernatant was stored at −80° C. until further assay. For BCAA determinations, a 20 μl perchloric acid supernatant aliquot of the mouse heart was thawed and neutralized with 0.25 M MOPS-3M KOH buffer for analysis of free amino acids in the heart. The amino acids were derivatized and extracted as previously described using internal and external standards (Wilson et al. (2011), American journal of physiology Endocrinology and metabolism. 301:E1236-E1242). The samples were analyzed using a Waters Synapt HDMS hybrid QTOF with Ion Mobility. BCKA measurements were performed as described elsewhere (Olson et al. (2013), supra). Briefly, the perchloric acid heart supernatants were derivatized by o-phenylenediamine (OPD), extracted with ethyl acetate, and dried down in glass tubes in an unheated vacuum centrifuge. Following drying, the ketoacids were reconstituted in 200 mM ammonium acetate, pH 6.8, and analyzed using a Shimadzu ultra-fast liquid chromatography (UFLC) 20ADXR LC system in-line with an AB-Sciex 5600 TripleTOF Q-TOF mass spectrometer (MS). Both instruments used in this analysis were housed in the Penn State College of Medicine Macromolecular Core Facility. BCKA concentration were measured and normalized to the weight of tissue. For human heart result, statistical analyses were performed with Student's t-test after log transforming the data.

BCKA Measurements in mouse Plasma: The method published by Olson et al. (2013), supra was followed with modification. Briefly, plasma was cleared of proteins by adding an equal volume of methanol, followed by two rounds of centrifugation. The supernatant was lyophilized and re-suspended in distilled water (dH20). Stock solutions of each keto acid were prepared in dH20 and stored at −80° C. until they were used once and not refrozen. 10 ng of [¹³C] KIV was added to each vial of sample and standard which were then derivatized with freshly prepared O-phenylenediamine (OPD) and extracted twice with ethyl acetate as described in Olson et al. (2013), supra. The pure organic phase was transferred to an eppendorf tube and dried under mild heat (40° C.). The samples were re-suspended in 50:50 MeOH:5 mM NH4 acetate and analyzed by LC-MS/MS using a Sciex 3200 Q-Trap coupled to a Shimadzu Prominence LC. An Agilent C18 XDB 5 micron packing column (50×4.6 mm) was used for chromatography with the following conditions: Buffer A: 5 mM NH4 acetate, Buffer B: methanol, 0-2.0 min 50% B, 2.0-2.5 min gradient to 100% B, 2.5-3.5 min 100% B, 3.5-3.6 min gradient to 50% B, 3.6-4.6 min 50% B. The derivatized keto acids were detected with the mass spectrometer in positive MRM (multiple reaction monitoring) mode using the following transitions: KIC 203.1 to 161.1 (retention time: 2.91 min); KIV 189.145 to 119.2 (retention time 2.84 min); KMV 203.065 to 174.2 (retention time 2.99 min); [¹³C] KIV 194.109 to 120.1 (retention time: 2.84 min).

Human Cohort for BCAA/BCKA Measurements

One hundred forty one subjects with no history of diabetes were selected from the CATHGEN bio-repository for ketoacid analysis based on cardiomyopathy phenotype. This study was approved by the Duke University Institutional Review Board. Heart failure (n=91) cases were defined as those having left ventricular ejection fraction (LVEF) less than or equal to 30%, no history of heart transplantation, no significant valvular disease, with or without history of myocardial infarction (MI), and New York Heart Association (NYHA) Functional Classification of 2 or greater. The control group (n=50) was composed of those with LVEF greater than or equal to 55%, no coronary vessels occluded greater than or equal to 50%, no history of MI, no history of percutaneous coronary intervention (PCI), no history of coronary artery bypass grafting (CABG), no history of congestive heart failure or heart transplantation, and no significant valvular heart disease.

Electron Spin Resonance Measurement of Superoxide Production

Freshly isolated hearts were placed into chilled modified Krebs/HEPES buffer (composition in mmol/l: 99.01 NaCl, 4.69 KCl, 2.50 CaCl₂, 1.20 MgSO₄, 1.03 KH₂PO4, 25.0 NaHCO₃, 20.0 Na-HEPES, and 5.6 glucose [pH 7.4]), cleaned of excessive adventitial tissue. The homogenates from heart tissues were prepared by homogenizing with a pestle (50 strokes) in fresh homogenization buffer (50 mmol/L of Tris-HCl, [pH 7.4] 0.1 mmol/L of EDTA, 0.1 mmol/L of EGTA) containing protease inhibitor cocktail and centrifuged at 800 g for 10 min. After centrifugation, supernatants were collected and then subjected for protein assay. The specific superoxide (O2.-) spin trap methoxycarbonyl-2,2,5,5-tetramethyl-pyrrolidine (CMH, 1 mmol/L, Alexis) solution was prepared freshly in nitrogen gas bubbled Krebs/HEPEs buffer containing diethyldithiocarbamic acid (DETC, 5 μmol/L Sigma) and deferoxamine (25 μmol/L, Sigma). Homogenates (15 μg protein) was then mixed with the spin trap solution in the presence or absence of 100 units of SOD (manganese containing enzyme, Sigma) and loaded into a glass capillary (Fisher Scientific). For analysis of O2.-signal (CM. formed after trapping O2.-), the capillary was immediately loaded in an e-Scan electron spin resonance (ESR) spectrophotometer (Bruker Biospin, Germany). The ESR settings used were static-field, 3484 sweep width, 9.00 G (1 G=0.1 mT); microwave frequency, 9.748660 GHz; microwave power 21.02 mW; modulation amplitude, 2470 mG; resolution in X, 512, number of X-scan, 10; and receiver gain, 1000. Data was presented as fold change versus WT. The superoxide production in isolated mitochondria was performed following a similar protocol except using an assay buffer containing 250 mM sucrose, 10 mM HEPES, 10 mM Tris-HCl (pH7.4), and 4 mM ADP.

Metabolomic Analysis of Heart Tissue

The global metabolomic analysis was carried out by Metabolon, Inc. (Durham, N.C.) using heart tissues from PP2Cm KO and wildtype male mice at 14-16 weeks of age. Briefly, all samples were quickly frozen in liquid nitrogen and maintained at −80° C. until processed. Samples were prepared using the automated MicroLab STAR® system from Hamilton Company. Several types of controls were analyzed in concert with the experimental samples. The LC-MS portion of the platform was based on a Waters ACQUITY ultra-performance liquid chromatography (UPLC) and a Thermo-Finnigan LTQ mass spectrometer operated at nominal mass resolution, which consisted of an electrospray ionization (ESI) source and linear ion-trap (LIT) mass analyzer. The samples destined for analysis by GC-MS were dried under vacuum prior to being derivatized under dried nitrogen using bistrimethyl-silyltrifluoroacetamide. Derivatized samples were separated on a 5% diphenyl/95% dimethyl polysiloxane fused silica column and analyzed on a Thermo-Finnigan Trace DSQ fast-scanning single-quadrupole mass spectrometer using electron impact ionization (EI) and operated at unit mass resolving power. Raw data was extracted, peak-identified and QC processed using Metabolon's hardware and software. Peaks were quantified using area-under-the-curve. A collection of information interpretation and visualization tools for use by data analysts. Welch's two-sample t-test is used to test whether two unknown means are different from two independent populations. Principal component analysis (PCA) was used to visualize how individual samples in the cohort of wildtype and PP2Cm KO hearts differ from each other. The metabolites contributing the greatest to the differences among the two groups were determined in a random forest (RF) analysis. To determine which metabolites make the largest contribution to the classification, the “Mean Decrease Accuracy” is computed.

Statistics

Unless otherwise specified, statistical analyses to compare 2 groups were performed with either the Student t test or the Wilcoxon rank-sum test (when n<5 or when the variance distributions differed on the basis of the Bartlett test). When >2 groups were analyzed, standard ANOVA followed by the Newman-Keuls test was performed when n>5 for all groups and passed by the Bartlett test of homogeneity of variances. Otherwise, the Kruskal-Wallis test was performed followed by the Dunn multiple-comparison test. Presented values are mean with standard deviation or standard error of the mean. A linear mixed-effect model test was performed for repeated measurements over time using lmerTest package in R obtained from an on-line source (Alexandra Kuznetsova, Per Bruun Brockhoff and Rune Haubo Bojesen Christensen (2015). lmerTest: Tests in Linear Mixed Effects Models. R package version 2.0-29. At the World Wide Web address of CRAN.R-project.org/package=lmerTest). A repeated measures linear model was fitted for echocardiograph parameters such as LVIDs and FS using animal ID as a random effect and day, group and group*day as fixed effects. A value of P<0.05 was considered statistically significant.

### Example 2: Catabolic Defect of Branched-Chain Amino Acids Promotes Heart Failure

Alterations in cardiac metabolism are hallmarks of the pathological changes in the failing heart, with studies over the past several decades centered on fatty acid and glucose utilization. Suppression of oxidative phosphorylation with reduced utilization of fatty acid in conjunction with increased glucose consumption is a common feature of heart failure. However, little is known about the metabolic changes of amino acid and their functional relevance in the pathogenesis of heart failure.

Amino acids serve as building blocks for protein synthesis and energy-providing substrates, although the relative importance of a bioenergetic contribution by amino acids in the heart remains unclear under either physiological or pathological conditions. In addition, derivatives of amino acids such as taurine, creatine, carnitine, and glutathione are critical to bioenergenesis and cellular function in the heart. An early study by Peterson et al. (J Mol Cell Cardiol. (1973) 5:139-147) suggested that total free amino acid concentrations were increased in the failing right ventricle. In patients with mitral valve disease, higher glutamine and glutamate concentrations were detected in the dilated left ventricle compared with the right ventricle. A metabolomic study has also demonstrated that intratissue concentrations of several amino acids were changed significantly in the failing rat heart (Kato et al. (2010) Circ Heart Fail. 3:420-430). More recently, 2 reports using multisystems analysis in hypertrophied and early-stage failing mouse hearts after pressure overload or myocardial infarction also revealed profound metabolic derangement, including amino acid metabolism, associated with pathological remodeling (Lai et al. (2014) Circ Heart Fail. 7:1022-1031; Sansbury et al. (2014) Circ Heart Fail. 7:634-642). These observations indicate that amino acid homeostasis is perturbed in diseased heart tissue.

In this study, it was found that the branched-chain amino acid (BCAA) catabolic pathway was the most significantly altered metabolic change in the mouse failing heart and that this coordinated suppression of BCAA catabolic pathway was regulated by Krüppel-like factor 15 (KLF15). Furthermore, it was found the loss of BCAA catabolic gene expression and the resulting accumulation of intramyocardial levels of BCAA catabolic mediators such as branched-chain α-keto acids (BCKAs) were conserved metabolic signatures in human failing hearts. Impairment of BCAA catabolic pathway impaired heart function and promoted pressure overload-induced heart failure, associated with elevated superoxide production, oxidative injury, and profound metabolic changes in the heart. Finally, pharmacological enhancement of BCAA catabolic activity significantly preserved cardiac function after pressure overload. These findings established that defect of BCAA catabolism is an underappreciated integral part of the metabolic reprogramming in stressed hearts, that amino acid metabolism makes a significant contribution to the progression of heart failure, and that the BCAA catabolic pathway can serve as a potential therapeutic target for the disease.

BCAA Catabolic Gene Regulation in Developing and Pathologically Stressed Hearts

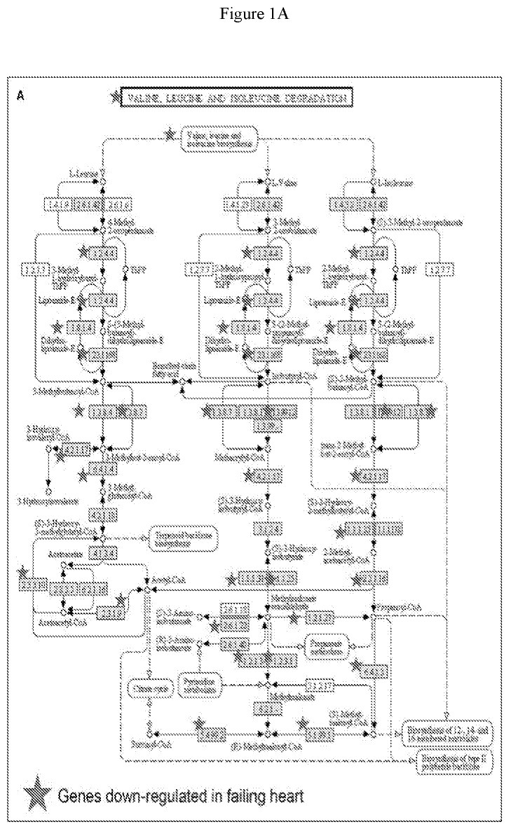

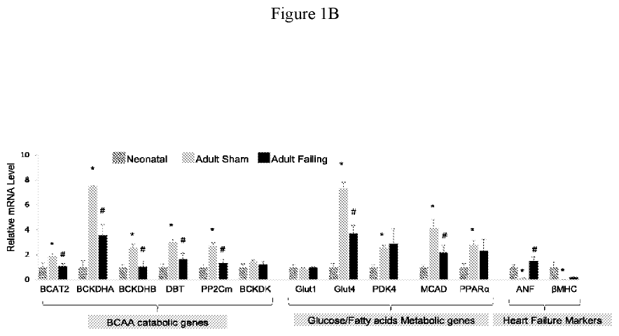

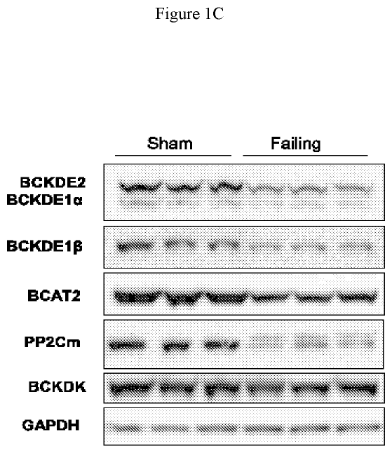

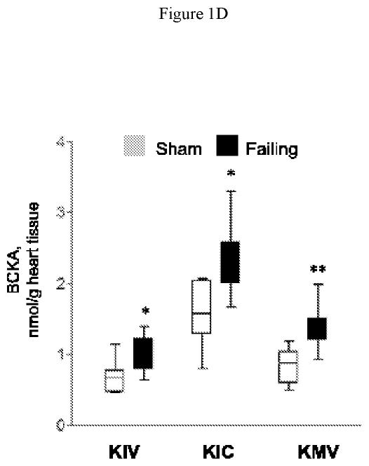

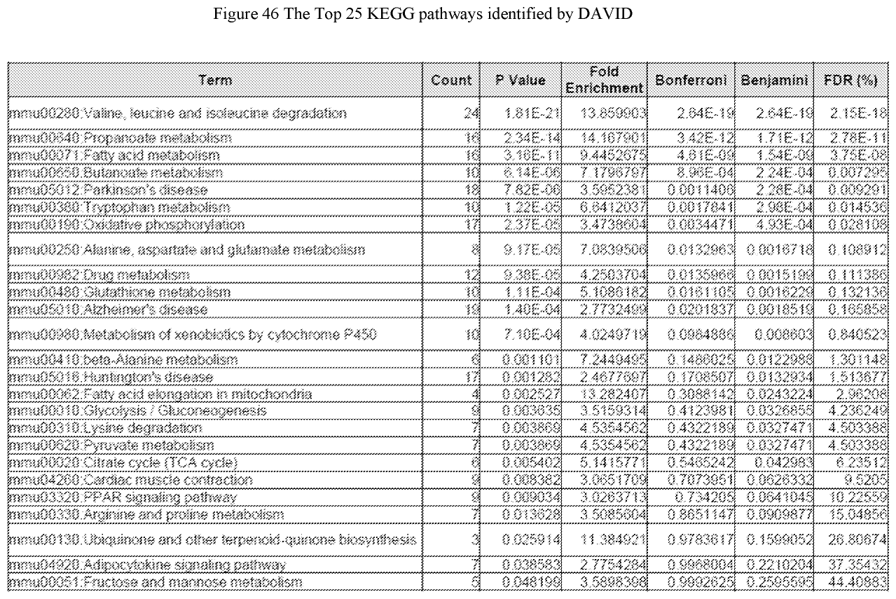

It has been well established that postnatal maturation of developing heart results in dynamic shifts from glucose to fatty acid utilization, a phenotype that is reversed in the diseased heart. These changes are orchestrated, at least in part, at the transcriptional level as part of the so-called fetal-like gene expression reprogramming. From cardiac transcriptome in pressure overload-induced failing mouse hearts, functional annotation analysis was performed using the Database for Annotation, Visualization and Integrated Discovery (at the World Wide Web address of david.abcc.ncifcrf.gov) to identify Kyoto Encyclopedia of Genes and Genomes pathways significantly overrepresented in differentially expressed genes. The analysis of downregulated genes in the failing heart revealed >20 specific metabolic pathways that were significantly enriched (FIG. 46). Unexpectedly, among them, the valine, leucine, and isoleucine (or BCAAs) catabolic pathway demonstrated the most significant changes associated with heart failure (FIG. 1A).

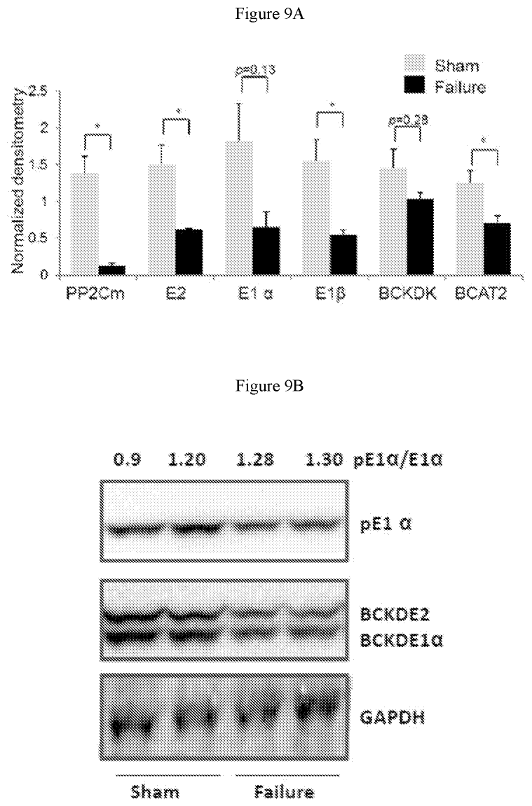

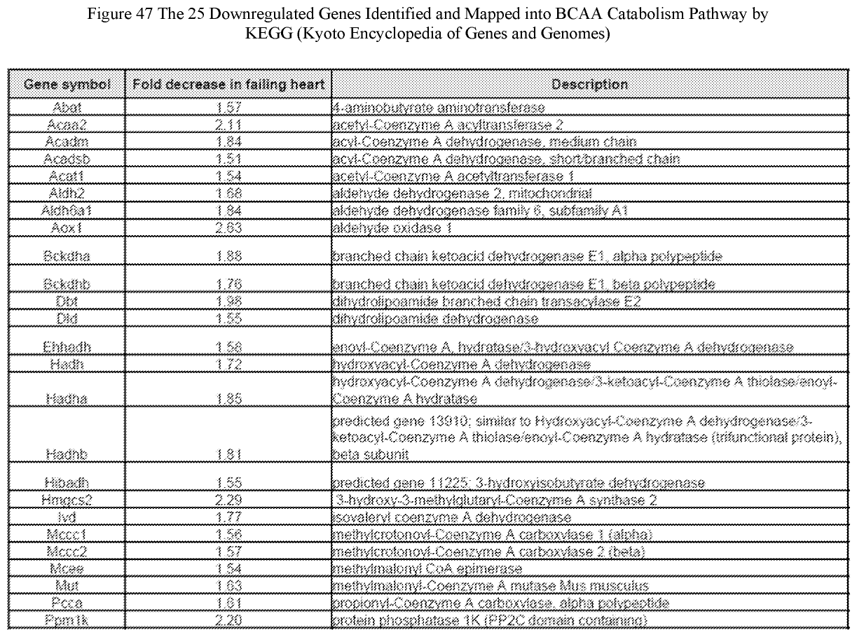

A total of 25 of 46 genes in the Kyoto Encyclopedia of Genes and Genomes BCAA catabolic pathway showed reduced expression in the failing heart compared with the sham controls (FIG. 1A and FIG. 47). The reduced expression of these key BCAA catabolic enzymes, including BCAT2, BCKD subunits E1α, E1β and E2, and the BCKD phosphatase PP2Cm, was verified at both the mRNA and protein levels (FIGS. 1B and 1C). In contrast, no reduction was seen in the expression of BCKD kinase (BCKDK; FIGS. 1B and 1C and FIG. 9A). However, a coordinated induction of the same set of genes during postnatal maturation from neonatal to adult was observed. This dynamic expression pattern is comparable to what is observed for glucose and fatty acid metabolic genes, including Glut4 (glucose transporter 4) and Mcad (medium-chain acyl-CoA dehydrogenase), along with other well established fetal-like marker genes, including Nppa and Myh7 (FIG. 1B). Therefore, the rate-limiting and downstream steps of the BCAA catabolic pathway are coordinately downregulated as part of the fetal-like transcriptome remodeling in failing heart.

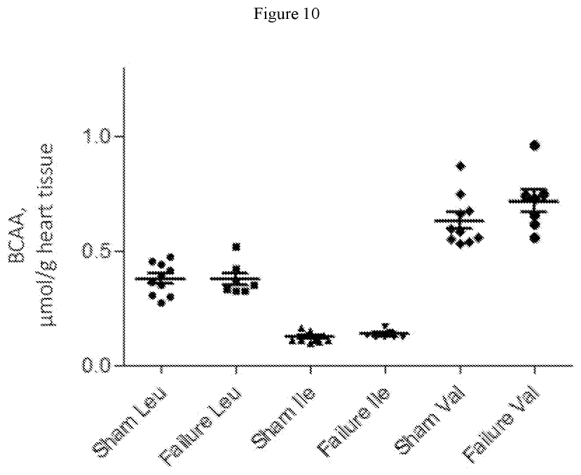

A significant reduction in PP2Cm expression along with an unchanged expression of BCKDK led to enhanced phosphorylation of BCKD regulatory subunit E1α in the failing hearts comparing with controls (FIG. 9B). Phosphorylation level of BCKD E1α subunit inversely correlates with the BCKD enzymatic activity; consequently, the levels of the intramyocardial BCKA were significantly increased in the mouse failing hearts (FIG. 1D), whereas the total BCAA levels remained unchanged (FIG. 10).

Defect of BCAA Catabolism in Human Failing Hearts

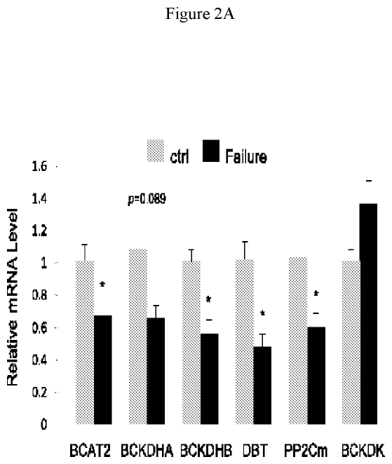

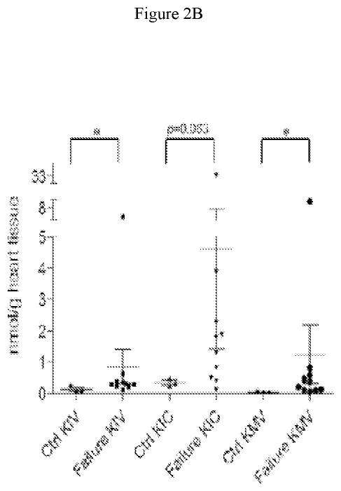

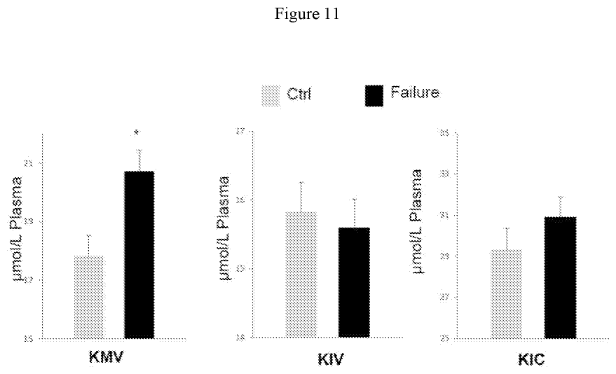

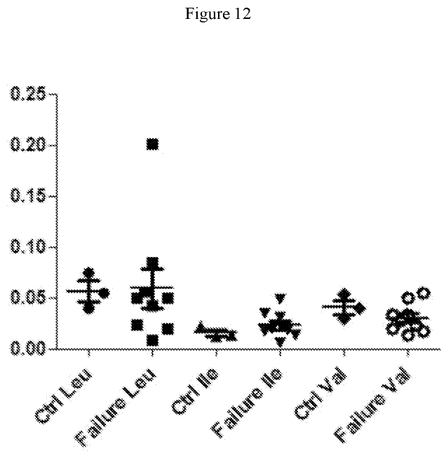

Human cardiomyopathy hearts demonstrated a striking parallel to the observations in rodents, with coordinated reduction of all key BCAA catabolic gene products, including BCAT2, BCKD subunits, and PP2Cm, whereas BCKDK expression was slightly increased (FIG. 2A). Importantly, intramyocardial levels of BCKA were also significantly increased in human cardiomyopathy hearts (FIG. 2B). A significantly higher level of KMV but not KIC or KIV was also observed in plasma from humans with heart failure (FIG. 11). In contrast, intramyocardial BCAA levels were not significantly altered (FIG. 12). Therefore, impairment of BCAA catabolic activity and intramyocardial accumulation of BCKA metabolites are conserved metabolic alterations in mouse and human failing hearts.

KLF15 Regulates Cardiac BCAA Catabolic Gene Expression

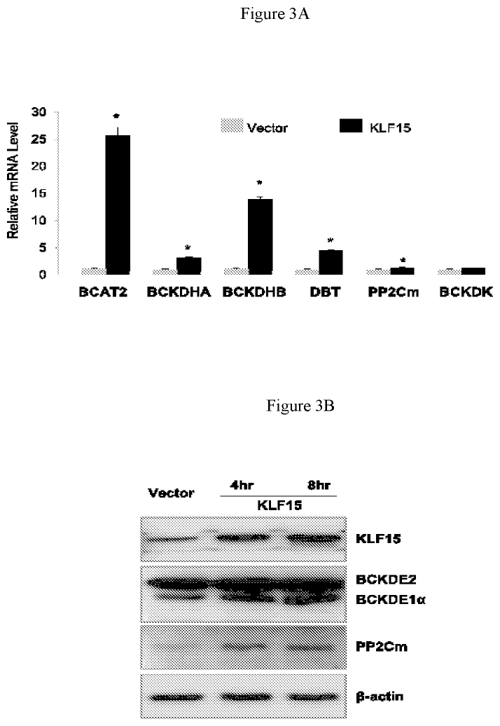

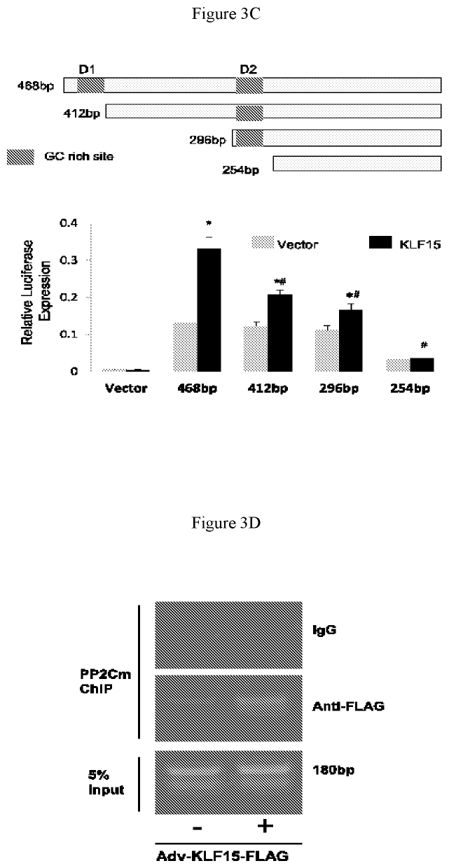

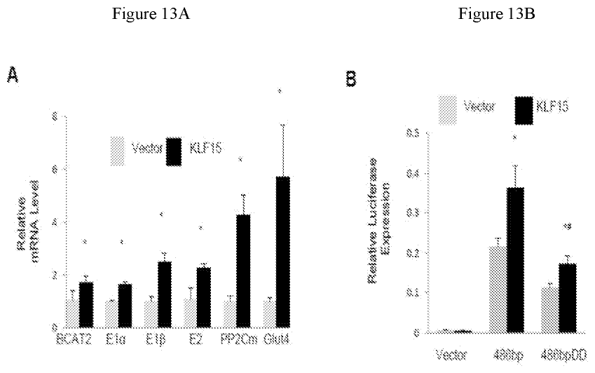

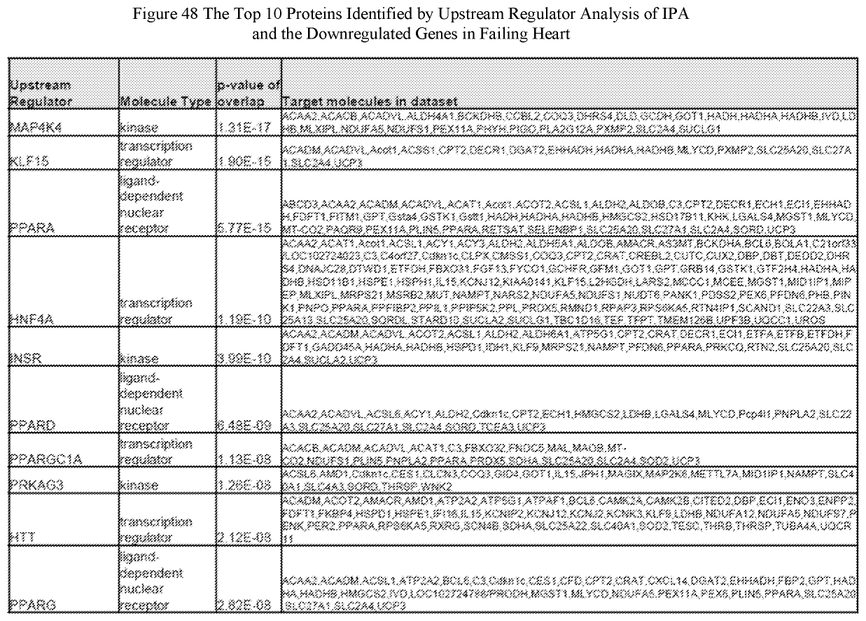

Coordinated regulation of BCAA gene products suggests a shared regulatory mechanism at the transcriptional level. An upstream regulator analysis was performed using Ingenuity Pathway Analysis software (at the World Wide Web address of www.ingenuity.com) for the genes showing altered expression in the mouse failing heart (Huang et al. (2008) Nat Protocols. 4:44-57). The analysis of downregulated genes in the failing heart predicted numerous factors involved in their regulation (FIG. 48). The top 3 candidates were MAP4K4, KLF15, and PPARA. KLF15 was reported to be a direct transcriptional activator of BCAT2 (Gray et al. (2007) Cell Metab. 5:305-312; Chaillou et al. (2013) J Appl Physiol (1985). 115:1065-1074; Zhou et al. (2012) J Biol Chem. 287:23397-23406). In cultured cardiomyocytes, overexpression of KLF15 significantly induced the mRNA expression of BCAT2, BCKD subunits, and PP2Cm, with a notable exception of BCKDK (FIG. 3A). Ectopic expression of KLF15 also induced the expression of these targets in non-myocytes (FIG. 3B and FIG. 13A). Using a PP2Cm promoter luciferase reporter, we showed that KLF15 directly induced the transcriptional activity of the PP2Cm promoter containing putative KLF15 binding motifs (FIG. 3C and FIG. 13B). Finally, chromatin immunoprecipitation analysis revealed a significant accumulation of KLF15 binding to the endogenous PP2Cm promoter in cardiomyocytes (FIG. 3D). Taken together, these data support a previously unidentified broad regulatory role for KLF15 in myocardial BCAA catabolic gene expression.

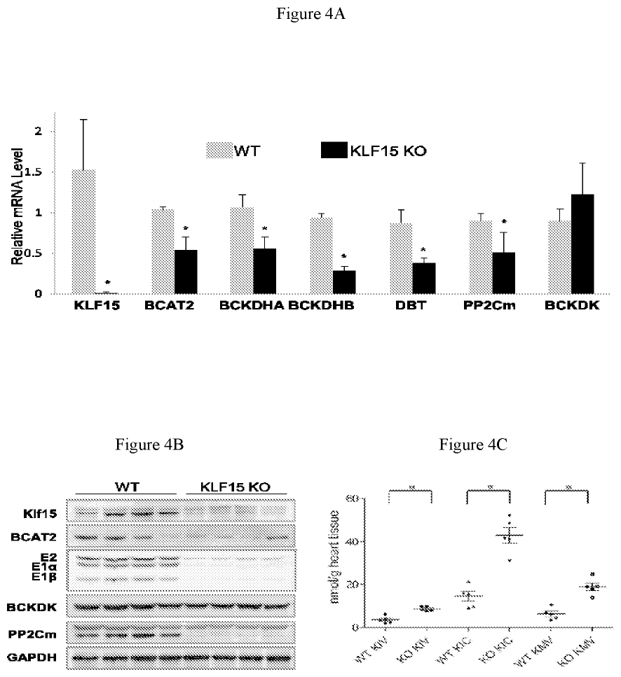

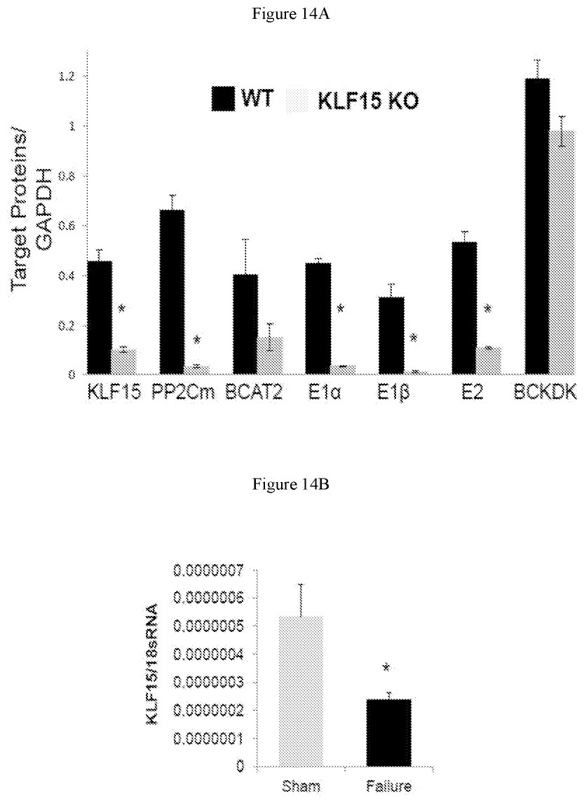

The abundance of BCAA catabolic genes was examined in Klf15-null hearts. Consistent with in vitro observations, the KLF15-deficient hearts displayed reduced expression of BCAT2, BCKD (E1a, E1b, E2), and PP2Cm, again with the notable exception of BCKDK, at both the mRNA and protein levels (FIGS. 4A and 4B and FIG. 14A), phenocopying what was observed in diseased mouse and human hearts (FIGS. 1 and 2). Also similar to the observed in failing human and mouse heart samples, elevated intramyocardial BCKA levels were identified in the Klf15-null hearts (FIG. 4C). Moreover, KLF15 expression was reduced in pressure-over-loaded murine hearts (FIG. 14B) and in human cardiomyopathy, as previously demonstrated (Haldar et al. (2010) Sci Transl Med. 2:26ra26; Prosdocimo et al. (2014) J Biol Chem. 289:5914-5924). Therefore, our data identify KLF15 as a central transcriptional regulator of the BCAA catabolic pathway and show that loss of KLF15 is a potential molecular mechanism underlying stress-induced BCAA catabolic defects in the diseased heart.

BCAA Catabolic Defect Impaired Cardiac Contractile Function

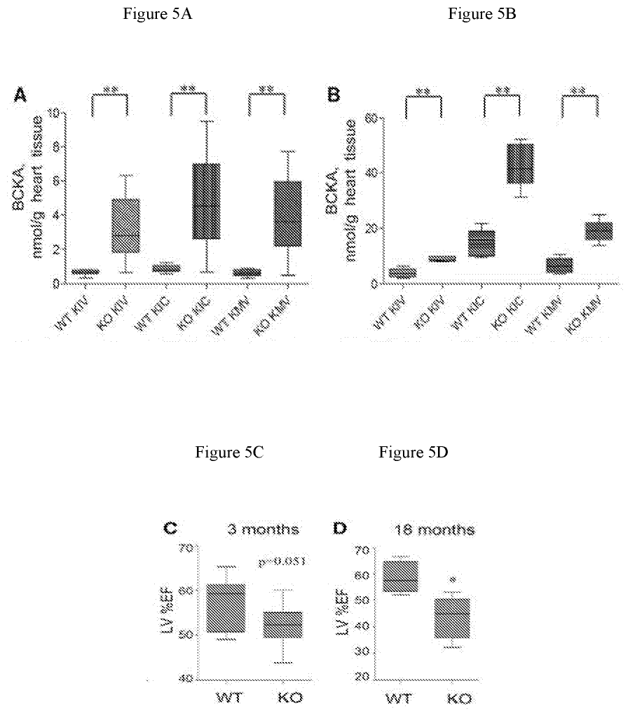

To directly assess the effect of BCAA catabolic defect on heart function, a mouse model is used carrying the genetically inactivated PP2Cm coding gene ppm1k (PP2Cm-KO) in which BCKD activity is significantly inhibited because of the constantly elevated E1α phosphorylation. Indeed, compared with wild-type controls, intramyocardial BCKA and BCAA levels were significantly increased in the PP2Cm-deficient hearts from mice fasted for 6 hours (FIG. 5A) at levels (<5 nmol·L⁻¹·g⁻¹) comparable to what was observed in mouse and human failing hearts (FIGS. 1D and 2B). However, cardiac BCKA concentrations became much higher (15-45 nmol·L⁻¹·g⁻¹) in the PP2Cm-KO heart under feeding conditions (FIG. 5B and FIG. 15A), highlighting the potential dietary influence on BCKA accumulation in the diseased heart when BCAA catabolic activity is compromised. Echocardiogram measurements showed a modest but statistically significant reduction in cardiac systolic function in the PP2Cm-deficient mice at 3 months of age (FIG. 5C). By 18 months of age, their cardiac function was further reduced compared with the age-matched wild-type controls (FIG. 5D). However, young PP2Cm-deficient mice exhibited no major changes in cardiac morphology, histology, and ultrastructure, as well as molecular markers of myocardial remodeling (FIG. 5E-5G and FIG. 15B). Therefore, abnormal BCAA catabolism is sufficient to promote contractile dysfunction over time in the absence of any external pathological stressor.

BCAA Catabolic Defect Enhances Susceptibility to Heart Failure in Response to Pathological Stress

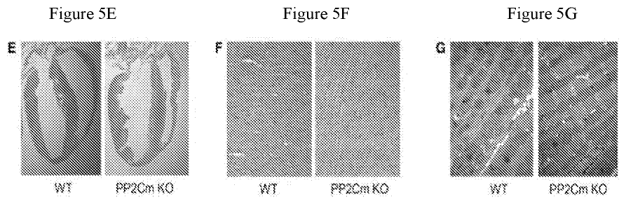

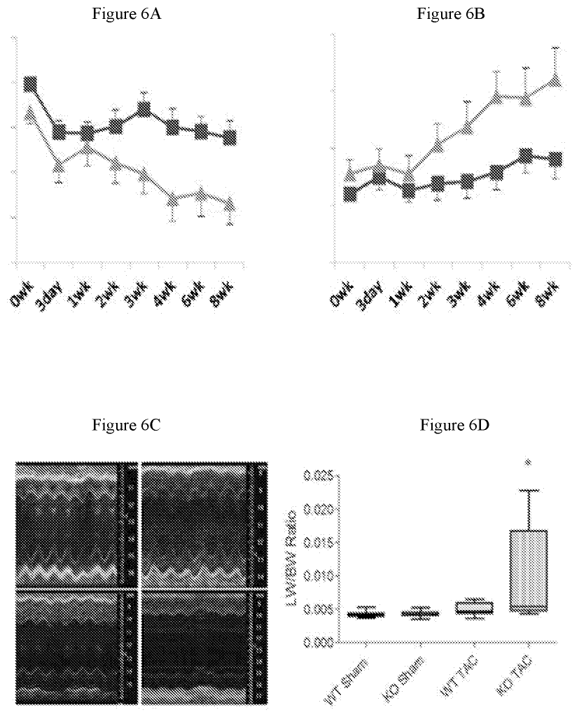

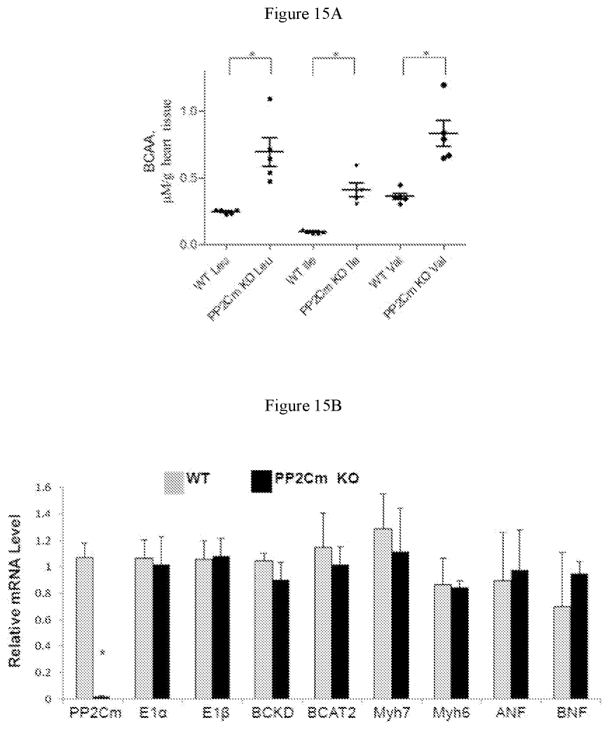

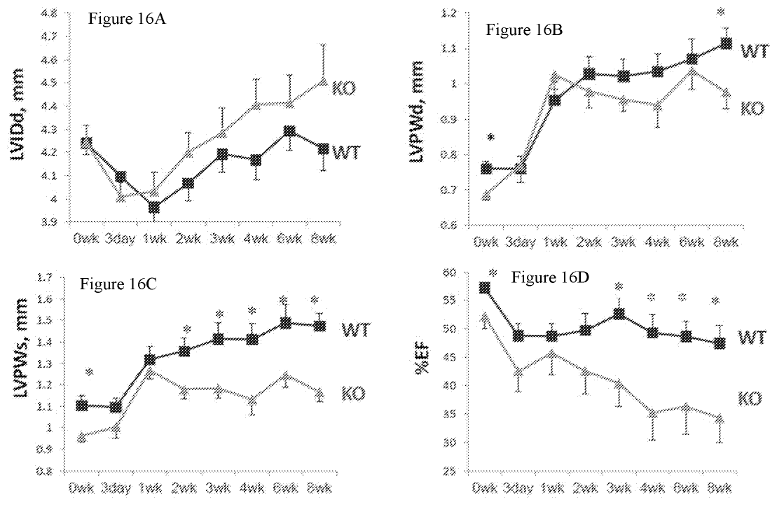

Wild-type and PP2Cm-KO mice (3-4 months of age) were subjected to pressure overload. From the second week of TAC, PP2Cm-deficient mice exhibited a marked reduction in contractile function (FIG. 6A and FIGS. 16A-16D). A repeated-measures linear model analysis demonstrated that changes in cardiac echocardiogram parameters such as left ventricular internal dimension in systole for group×day (P=3.54e⁻⁶) and left ventricular fractional shortening (P=1.27e⁻³) were significant between the PP2Cm-deficient and wild-type mice. At 8 weeks after TAC, PP2Cm-deficient mice displayed signs of heart failure, as evidenced by significantly reduced left ventricular ejection fraction, chamber dilation, and elevated wet lung weights, an indicator of severe pulmonary congestion resulting from heart failure (FIG. 6). Collectively, these data indicate that deficient BCAA catabolism can directly impair cardiac function and accelerates pressure overload-induced cardiomyopathy.

Impaired Metabolic and Redox Homeostasis by BCAA Catabolic Deficiency in Heart

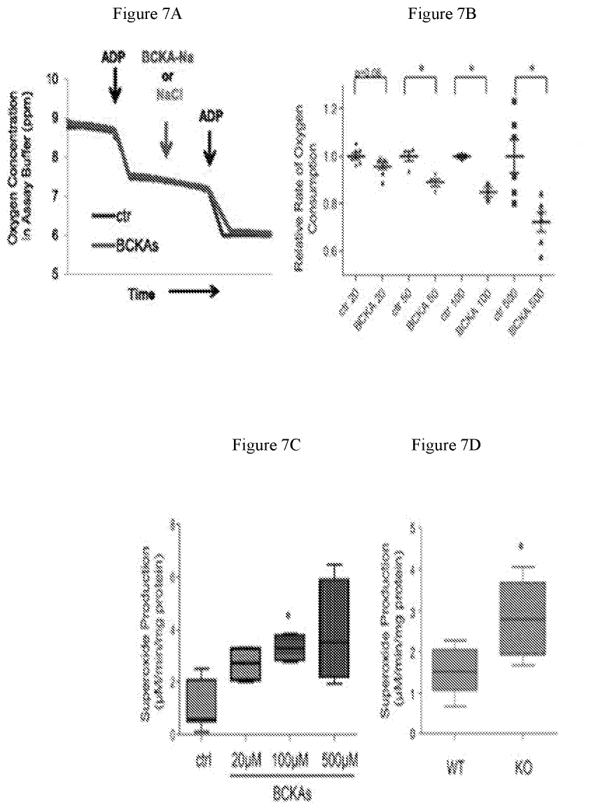

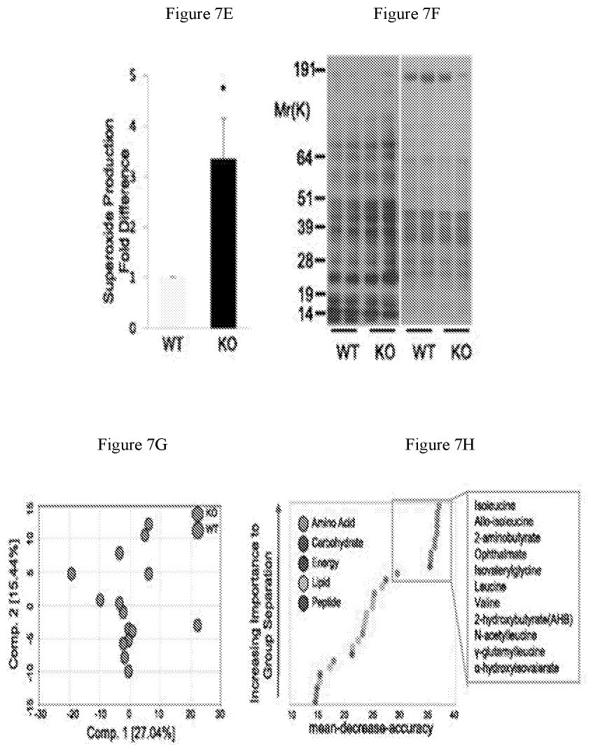

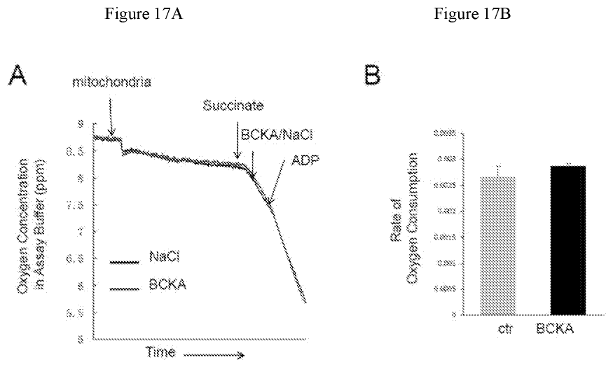

The impact of BCAA catabolic defects on cardiac function is consistently correlated with elevated BCKA metabolites in the diseased heart tissue. It was found that in isolated cardiac mitochondria, BCKAs directly inhibited complex I—but not complex II—mediated respiration (FIG. 7A and FIGS. 17A and 17B). The inhibition was dose dependent with a marked decrease observed at concentrations as low as 20 μmol/L BCKAs (FIG. 7B). In the meantime, BCKAs also promoted superoxide production in isolated cardiac mitochondria in a dose-dependent manner (FIG. 7C). A significant increase in superoxide production was detected from the PP2Cm-deficient mitochondria (FIG. 7D) and myocardium (FIG. 7E), associated with the enhanced oxidative injury to cardiac proteins (FIG. 7F). These data suggest that accumulated BCKAs resulting from BCKD inactivation may directly affect cardiac mitochondrial activity and redox homeostasis.

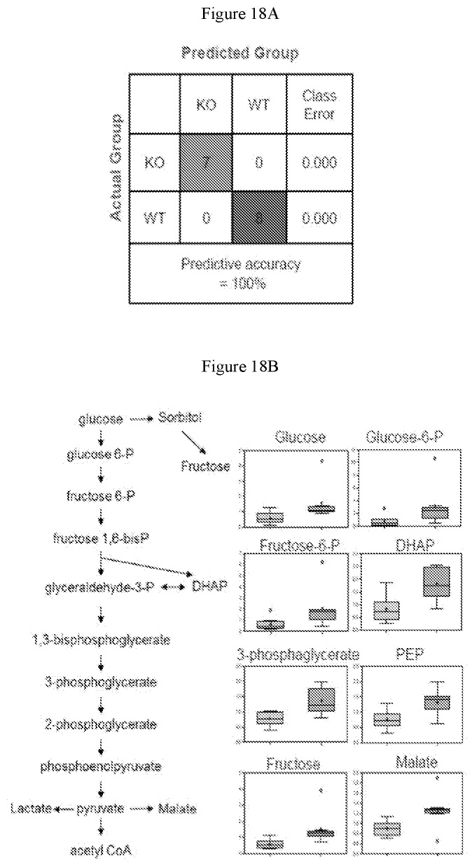

Additional targeted metabolomic analysis were performed on >300 metabolic intermediates in hearts from wild-type and PP2Cm-KO mice. Principal component analysis revealed a divergent separation between wild-type and PP2Cm-KO hearts (FIG. 7G), suggesting global metabolic changes associated with BCKD inhibition. When random forest analysis was applied to cluster all samples, changes in intracardiac metabolites separated the wild-type and PP2Cm-KO mice with 100% predictive accuracy (FIG. 18A). In addition to BCAA and their metabolites, the top 30 most significantly changed metabolites in the PP2Cm-KO hearts include lipids and carbohydrates (FIG. 7H). Specifically, the levels of glucose, glycolytic intermediates, and glucose-derived sugars such as fructose and mannose 6-phosphate were markedly elevated in the PP2Cm-deficient heart (FIG. 18B). These results support the notion that BCAA catabolic deficiency and elevated BCKA can result in impaired mitochondrial function, reactive oxygen species induction, and global perturbations in the myocardial metabolic profile.

Inhibition of BCKDK Promoted BCKA Degradation and Preserved Heart Function

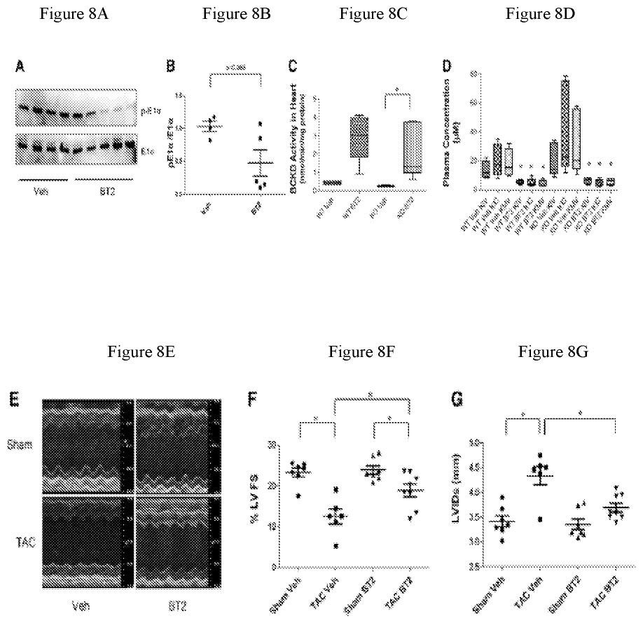

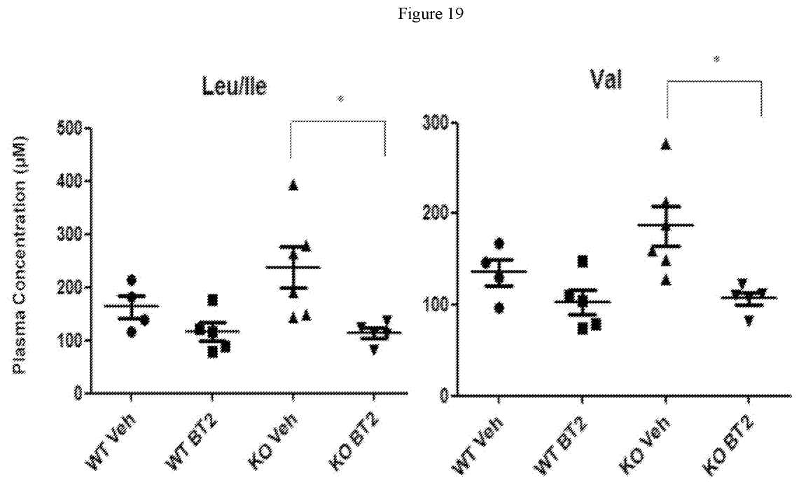

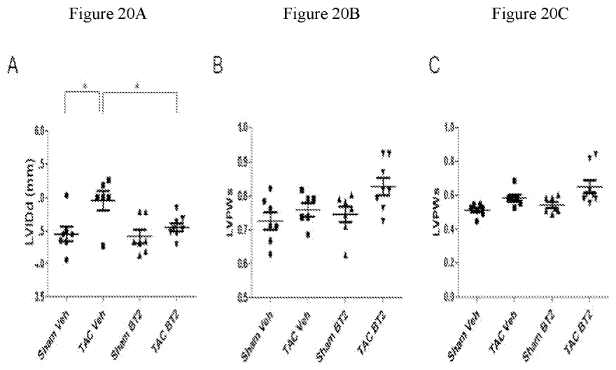

Given the significant contribution of BCAA catabolic defect to cardiac dysfunction, the impact of enhancing BCAA catabolic activity on pressure over-load-induced heart failure was investigated in mice. 3,6-Dichlorobenzo[b] thiophene-2-carboxylic acid (BT2) is a highly specific and potent inhibitor of BCKDK (Olson et al. (2013), supra). Administration of BT2 in mice significantly reduced the phosphorylation of BCKD subunit E1α in heart (FIGS. 8A and 8B) and dramatically enhanced cardiac BCKD activity in both wild-type (˜7-fold) and the PP2Cm-KO mice (˜9-fold; FIG. 8C). Consequently, the plasma BCKA level in both wild-type and PP2Cm-KO mice was markedly reduced (FIG. 8D) but with modest impact on plasma BCAA level (FIG. 19). More important, at 4 weeks after TAC, BT2-treated mice displayed significantly preserved left ventricular ejection fraction and reduced chamber dilation (FIG. 8D-8F and FIG. 20). These results suggested that enhancing BCKA degradation by targeted inhibition of BCKDK significantly preserved cardiac function in response to pathological stress.

Discussion

The present study reveals that BCAA catabolic gene expression is coordinately suppressed in both murine and human failing hearts as part of fetal-like gene expression and metabolic reprogramming. KLF15-mediated transcriptional regulation is central for this coordinated reduction of BCAA catabolism. Genetic and cellular analyses suggest that BCAA catabolic defects and the resulting accumulation of BCKA metabolites cause cardiac reactive oxygen species injury and global metabolic alteration and significantly contribute to the progression of heart failure.

Amino acids serve as both important nutrients and potent signaling molecules. However, compared with the extensive knowledge of fatty acid and glucose metabolism, current understanding of amino acid metabolic regulation under normal development or pathological conditions is very limited. BCAAs, including leucine, isoleucine, and valine, are essential amino acids with a shared catabolic pathway. In addition to participating in de novo protein synthesis, BCAAs function as potent nutrient signal molecules for cellular metabolism and growth. Through the mechanistic target of rapamycin pathway, BCAAs (particularly leucine) can regulate vital cellular processes, including protein translation, autophagy, and insulin signaling (Melnik (2012) World J Diabetes. 3:38-53), affecting glucose and fatty acid metabolism (Vary and Lynch (2007) J Nutr. 137:1835-1843), muscle anabolism (D'Antona et al. (2010) Cell Metab. 12:362-372), and life span (Barschak et al. (2008) Clin Biochem. 41:317-324). Genetic defect of BCAA catabolism leads to maple syrup urine disease (Mochel et al. (2007) PLoS One. 2:e647). Recently, abnormal plasma BCAA levels have been associated with neurological, cardiovascular, metabolic diseases, and cancer in numerous studies (see, e.g., Mayers et al. (2016) Science 353:1161-1165 and Mayers et al. (2014) Nat. Med. 20:1193-1198). These findings highlight the importance of BCAA metabolism in normal physiology and a broad spectrum of human diseases. Suppressed BCAA catabolic activity appears to be a common feature in the stressed heart. Earlier reports by Kato et al. (2010), supra using Dahl salt-sensitive rats demonstrate that cardiac valine, isoleucine, and leucine levels are elevated after a high-salt diet. Several other studies, including our present study in both rodents and humans, have now linked high levels of BCAAs with cardiac diseases. Therefore, BCAA catabolic defect is another metabolic hallmark of heart diseases that may be exploited as additional metabolic biomarkers for cardiac pathology.

The coordinated loss of BCAA catabolic gene expression suggests a common regulatory machinery for the pathway. This notion is consistent with 2 recent studies reported in hypertrophied and early-stage failing heart (Lai et al. (2014), supra; Sansbury et al. (2014), supra). From both bioinformatic and genetic approaches, this present study identified KLF15 as a master transcription factor responsible for BCAA catabolic gene expression in heart. The functional role of KLF15 is well documented in cardiac hypertrophy, heart failure, and cardiac fibrosis. In addition to hypertrophic genes, KLF15 serves as a key regulator of glucose, fatty acid, and amino acid metabolism. KLF15 is reported to directly modulate the expression of BCAT2 as a mechanism to modulate mechanistic target of rapamycin signaling in skeletal muscle. KLF15 has previously been shown to be regulated by diverse pathological stimuli. Human and murine forms of pressure-overload cardiomyopathy have been shown to reduce KLF15 levels, a result in humans that is reversed by mechanical unloading. Moreover, hypertrophic stimuli (including angiotensin II, phenylephrine, and endothelin-1) have been shown to reduce KLF15 levels both in vivo and in vitro. Our data reinforce the notion that KLF15 is an important regulator for metabolic reprogramming in heart by modulating several important branches of macronutrient metabolism, including fatty acid, glucose, and amino acids.

It is intriguing that metabolic profiling in hypertrophic (1 week after TAC) or post-myocardial infarction hearts revealed elevated BCAA concentrations in cardiac tissue, in contrast to what we observed in both end-stage human cardiomyopathy hearts and mouse failing hearts 8 weeks after TAC. It is plausible to speculate that BCAA catabolic reprogramming is a compensatory mechanism at least at the initial stage of the response of the myocardium to stress, given that BCAA preservation would redirect amino acids from catabolic consumption to protein synthesis and cell growth during cardiac hypertrophy. Perturbation of BCAA catabolic activity may have a significant impact on mechanistic target of rapamycin signaling, leading to potential changes in cardiac growth, metabolism, and survival. However, defective BCKD activity also causes accumulation of BCKA in hearts, which may lead to a detrimental effect resulting from cytotoxic effects on mitochondrial function and reactive oxygen species homeostasis. Indeed, a direct and dose-dependent impact of BCKA treatment on mitochondrial function and reactive oxygen species production as demonstrated in this study highlights the potential contribution of BCKA as the true pathogenic culprit underlying BCAA catabolic defects in the progression of heart failure. BCKA and BCKA-mediated mitochondrial and cellular defects should be further explored as both metabolic biomarkers and therapeutic targets for heart failure.

The present genetic data from this report clearly implicate BCAA catabolic defect as a significant contributor to the pathogenesis of heart failure. The results from this study using BCKDK inhibitor (FIG. 8) clearly demonstrate the translational value of targeting BCAA catabolism as a therapy for heart failure.

For a detailed report of this study, see Sun et al. (2016) Circulation 133:2038-2049, which is incorporated herein by reference to its entity.

### Example 3: Exemplary Materials and Methods Used in Example 4

Animals

Ob/ob mice or wildtype C57BL/6 male mice were purchased from Jackson Labs or SLAC Laboratory Animals Company Limited, Shanghai, China. PP2Cm germ-line knockout mice were generated as previously described (Lu et al. (2009) J Clin Invest 119:1678-1687). All animals were housed at 22° C. with a 12-hour light, 12-hour dark cycle with free access to water and standard chow. BCAA in water was prepared by BCAA powders. Isocaloric high protein diet (TD90018) and low protein diet (TD90016) were purchased from Envigo Teklad Diet. For glucose or insulin tolerance test, mice were fasted for 6 hours and injected (intraperitoneally) with insulin or D-glucose. Assays for the BCKD activity were carried out as prescribed previously (Tso et al. (2014) Journal of Biological Chemistry 289:20583-20593). Compound BT2 (3,6-dichlorobenzo[b]thiophene-2-carboxylic acid) was administrated by oral gavage at 40 mg/kg/day. For BCAA tolerance test, ob/ob mice at age of 10 weeks or high fat diet induced obese (DIO) mice were used. To induce DIO, high fat diet (60% kcal % fat) or control diet (10% kcal % fat) (Research Diets, New Brunswick, N.J.) was used to feed mice starting at 6 weeks of age and continued for 8 weeks. Obese mice were fasted for 6 hours and then i.p. administered a 150 mM leucine solution at the dose of 15 μl/g body weight. Plasma samples were collected at 0, 30, 60, 120 minute points to measure BCAA/BCKA levels. All animal procedures were carried out in accordance with the guidelines and protocols approved by the Committee for Humane Treatment of Animals at Shanghai Jiao Tong University School of Medicine, the University of Texas Southwestern Medical Center Institutional Animal Care and Use Committee, or the University of California at Los Angeles Institutional Animal Care and Use Committee.

Diets for Mouse Studies

45% kcal % fat high fat diet or control diet (10% kcal % fat) were purchased from ResearchDiets (New Brunswick, N.J.) and used to feed mice at 6 weeks of age for 8 weeks to induce obesity. BCAA in water was prepared by BCAA powders. High protein diet (TD90018) and low protein diet (TD90016) were purchased from Envigo Teklad Diet.

Mouse Treatment with BCKDK Inhibitor BT2

Obese mice (ob/ob mice at age of about 10 weeks or wildtype mice on HFD for about 10 weeks) were randomized into two groups for vehicle and BT2 treatment. BT2 was dissolved in DMSO and diluted into 5% DMSO, 10% cremophor EL, and 85% 0.1 M sodium bicarbonate, pH 9.0, for delivery. Animals were dosed daily by oral gavage with BT2 solution (40 mg/kg/day) or equal volume of vehicle for 8-10 weeks. Animals were euthanized using carbon dioxide asphyxiation followed by cervical dislocation and then dissected. Blood was harvested by cardiac puncture and centrifuged to isolate plasma which was stored at −80° C. Acidified citrate dextrose was used as an anticoagulant. Immediately after blood collection, heart, liver, kidneys, and both hind leg quadriceps muscles were removed and snap frozen in liquid nitrogen.

Assays for BCKD Activity in Mouse Tissues

Assays for the BCKD activity were carried out as prescribed previously (Tso et al. (2014), supra). Briefly, frozen tissues were thawed and homogenized in a homogenizing buffer (30 mM KPi, pH 7.5, 3 mM EDTA, 5 mM DTT, 1 mM KIC, 3% fetal bovine serum, 5% Triton X-100, and 1 μM leupeptin). Samples were spun at 25,000×g and supernatants were collected. Diluted samples were placed in 24-well assay plates with assay buffer (30 mMKPi, pH7.5, 0.4 mM CoA, 3 mM NAD, 5% fetal bovine serum, 2 mM thiamine diphosphate, 2 mM MgCl2, and 65 μg of human E3). Keto [1-¹⁴C] isovalerate substrate was added and assay plates were incubated at 37° C. for 30 min. 20% TCA solution was added to stop the reactions. Assay plates were incubated further at 37° C. for 45 min. ¹⁴CO2 trapped on 2 M NaOH soaked filter wicks was counted in a liquid scintillation counter.

Determination of BCAA/BCKA Concentrations

Plasma valine and leucine/isoleucine concentrations were determined by LC-MS/MS in a 4000 Qtrap mass spectrometer coupled to a Shimadzu Prominence LC as described previously (Tso et al. (2013) P.N.A.S. USA 110:9728-9733). In the present analysis, valine and leucine/isoleucine standard curves were prepared by spiking known concentrations of each amino acid into murine plasma. The endogenous signal from blank plasma was subtracted from each point on the standard curve, and the data were plotted in GraphPad Prism. BCAA concentrations in treated samples were determined based on this standard curve.

Leucine Challenge Test

Lean male wild type and obese (ob/ob or high-fat-diet-induced) mice at age of 14 weeks were fasted for 6 hours and then i.p. administered a 150-mM leucine solution at the dose of 15 μl/g body weight. Blood samples were collected at 0, 30, 60, 120 minute points as indicated. Plasma was then collected and BCAA/BCKA was measured.

Chemicals and Agents

BCAA, BCKA, insulin, and D-glucose were purchased from Sigma (St. Louis, Mo.). Adeno-GFP-Sfrp5 viruses were generated previously and used at 1×108 PFU per mouse (Guan et al. (2016) 10.1530/joe-15-0535).

Glucose and Insulin Tolerance Test

Mice were fasted for 6 hours. For insulin tolerance test, mice were injected (intraperitoneal) with insulin (0.75 U/kg body weight). For glucose tolerance test, mice were injected intraperitoneally with D-glucose (2 g/kg body weight). Experiments were performed between 14:00 and 16:00. Blood was drawn 0, 15, 30, 60, and 120 min after insulin or glucose administration. Blood glucose concentrations were measured through tail bleeding before and at the times indicated after injection. Blood glucose was measured using a portable glucometer (Johnson&Johnson). Plasma insulin and TNFα concentrations were measured by Milliplex Multiplex Immunoassay Kits MMHMAG-44K-14 (Merck Millipore, Darmstadt, Germany).

Western Blot Analysis

Proteins from tissue or cells were harvested in buffer (50 mM HEPES, pH7.4, 150 mM NaCl, 1% NP-40, 1 mM EDTA, 1 mM EGTA, 1 mM glycerophosphate, 2.5 mM sodium pyrophosphate, 1 mM Na3VO4, 20 mM NaF, 1 mM phenylmethylsulfonyl fluoride, 1 μg/mL of aprotinin, leupeptin, and pepstatin). Samples were separated on 4-12% Bis-Tris gels (Invitrogen, Carlsbad, Calif.), and transferred onto a nitrocellulose blot (Amersham, Little Chalfont, UK). The blot was probed with the indicated primary antibodies. Protein signals were detected using HRP conjugated secondary antibodies and enhanced chemiluminescence (ECL) western blotting detection regents (Pierce, Dallas, Tex.). Rabbit polyclonal antisera against the E1α and E1β subunits are a kind gift from Dr. Yoshiharu Shimomura (Nagoya Institute of Technology, Nagoya, JP). pE1α antibody (NBP2-04023) was purchased from Novus Biologicals (Littleton, Colo.). The BCKDK antibody (AV52131) was purchased from Sigma. Phospho-Akt (Ser473), Akt, and GAPDH antibodies were purchased from Cell Signaling Technology (Denver, Mass.). β-actin antibody were purchased from Sigma Chemical Co. The polyclonal antibody against mouse Sfrp5 was purchased from Santa Cruz Biotechnology (Dallas, Tex.) or Abcam (ab198206) (Cambridge, Mass.).

RNA Isolation and qRT-PCR

Total RNA was extracted using the Trizol (Invitrogen) according to the manufacturer's instructions. Total RNA (2 μg) was reverse transcribed using random primers and MMLV (Promega, Madison, Wis.). Each cDNA sample was analyzed in triplicate with the Applied Biosystems Prism 7900HT Real-Time PCR System using Absolute SYBR Green (ABI, Sterling, Va.). The relative amount of specific mRNA was normalized by 18sRNA. Sfrp5 primer sequences are: forward, 5′-TCCTCTGCTCGCTCTTCGCT-3′ (SEQ ID NO:3) and reverse, 5′-CCAATCAACTTTCGGTCCCC-3′ (SEQ ID NO:4).

Metabolomic Analysis

The global metabolomic analysis was carried out by Metabolon, Inc. (Durham, N.C.) using liver, white adipose tissues and plasma from male mice at 14-16 weeks of age, ob/ob mice at 14 weeks. Briefly, all samples were quickly frozen in liquid nitrogen and maintained at −80° C. until processed. Samples were prepared using the automated MicroLab STAR® system from Hamilton Company (Reno, Nev.). Several types of controls were analyzed in concert with the experimental samples. The LC-MS portion of the platform was based on a Waters ACQUITY ultra-performance liquid chromatography (UPLC) and a Thermo-Finnigan LTQ mass spectrometer operated at nominal mass resolution, which consisted of an electrospray ionization (ESI) source and linear ion-trap (LIT) mass analyzer. The samples destined for analysis by GC-MS were dried under vacuum prior to being derivatized under dried nitrogen using bistrimethyl-silyltrifluoroacetamide. Derivatized samples were separated on a 5% diphenyl/95% dimethyl polysiloxane fused silica column and analyzed on a Thermo-Finnigan Trace DSQ fast-scanning single-quadrupole mass spectrometer using electron impact ionization (EI) and operated at unit mass resolving power. Raw data was extracted, peak-identified and QC processed using Metabolon's hardware and software. Peaks were quantified using area-under-the-curve. A collection of information interpretation and visualization tools for use by data analysts. Welch's two-sample t-test is used to test whether two unknown means are different from two independent populations.

Human GWAS for Insulin Related Traits

Publicly available human GWAS datasets for fasting glucose, fasting insulin and insulin resistance (BMI unadjusted and adjusted) were retrieved from large meta-analysis consortia including MAGIC (the Meta-Analyses of Glucose and Insulin-related traits Consortium) (Dupuis et al. (2010) Nature genetics 42:105-116) and GENESIS (GENEticS of Insulin Sensitivity consortium) (Knowles et al. (2015) Journal of Clinical Investigation 125:1739-1751). After retrieving summary level statistics for all single nucleotide polymorphisms (SNPs), SNPs with weak association (lower 80%) were removed. Remaining SNPs with high linkage disequilibrium (LD r²>0.5) were filtered using a previously described method (Makinen et al. (2014) PLoS genetics 10:e1004502). LD data for European ancestry was from Hapmap3 (International HapMap Consortium (2003) Nature 426:789-796) and 1000 Genomes Project (1000 Genomes Project Consortium (2012) Nature 491:56-65).

Tissue-Specific Expression Quantitative Trait Loci (eQTLs)

Comprehensive lists of human cis-eQTLs from human adipose, liver and muscle tissues are accessible from the Mergeomics webserver (Arneson et al. (2016) BMC genomics 17:722).

Tissue-Specific Co-Expression Modules

A total of 1689 coexpression modules were constructed from adipose, liver and muscle tissue samples generated in multiple human and mouse studies using WGCNA (Langfelder et al. (2008) BMC bioinformatics 9:559).

Amino Acid Pathways and Other Canonical Pathways

BCAA catabolism and eleven non-BCAA amino acid pathways were retrieved from the KEGG database (Kanehisa and Goto (2000) Nucleic Acids Res 28:27-30). Due to the key regulatory role of PPM1K and BCKDK, they were manually added into the KEGG BCAA pathway. BCAA pathway was further categorized into groups of genes specific to the degradation of leucine, valine and isoleucine. For non-BCAA amino acid pathways, overlapping genes with BCAA were removed. Bonferroni corrected p<0.05, fold enrichment>5, and number of overlapping genes>2 were used as the cutoffs to determine significance when annotating co-expression modules.

Integrative Genomics Analysis Using the Mergeomics Pipeline

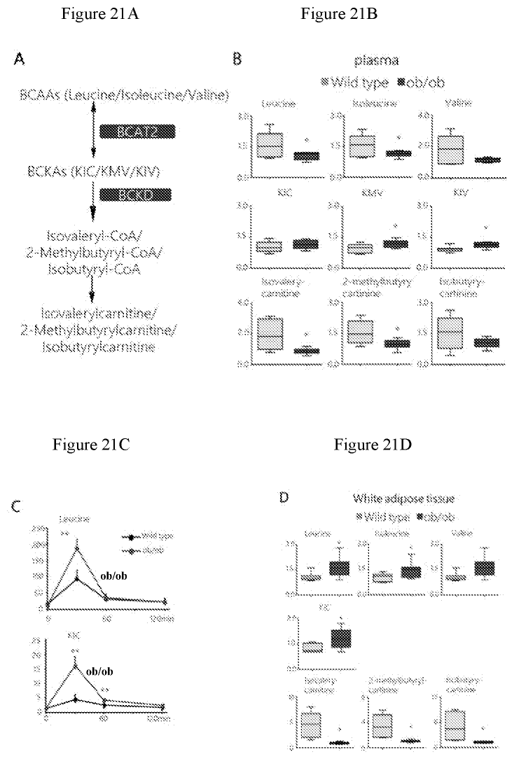

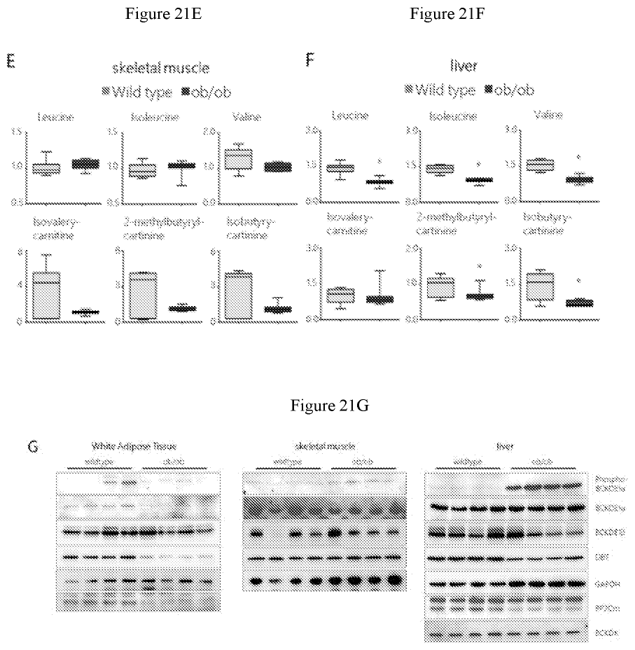

The Mergeomics pipeline is an R-based computational framework for the integration of multi-dimensional datasets (Shu et al. (2016) BioRviv doi: https://doi.org/10.1101/036012). The Marker Set Enrichment Analysis (MSEA) library in Mergeomics was used to determine the association of co-expression networks with human insulin traits by leveraging human GWAS and eQTLs (FIG. 21A). Specifically, co-expression modules were first mapped to adipose, liver and muscle eQTLs to derive the corresponding representative expression single nucleotide polymorphism (eSNP) sets. The disease association p values of the eSNPs were then extracted from the filtered summary level statistics as described above. Significance of enrichment for moderate to strong risk SNPs for a trait within each module was assessed using a chi-square like statistic followed by multiple testing correction to estimate FDR, as described in detail in Shu et al. (2016), supra. Modules at FDR<5% and p<0.05 were considered to be significantly or suggestively associated with the respective trait.

To test if BCAA genes play key regulatory role in the trait-associated BCAA modules, the weighted Key Driver Analysis (wKDA) library in Mergeomics was used to identify key drivers (KD) of the significant modules. KDs of a given module are defined as genes whose neighboring subnetwork exhibited significant enrichment (FDR<1%) for member genes in that module. Adipose Bayesian networks used in KDA were constructed through a previously developed method (Zhu et al. (2007) PLoS computational biology 3:e69; Zhu et al. (2008) Nature genetics 40:854-861).

Gene-Trait Correlation in Hybrid Mouse Diversity Panel (HMDP)

Pearson correlations of the expression levels of BCAA genes in liver, adipose, and muscle tissues with fasting glucose, fasting insulin and HOMA-IR measurements were retrieved from the HMDP panel (at the World Wide Web address of systems.genetics.ucla.edu/data/hmdp) comprised of ˜100 mouse strains fed with high-fat diet (Bennett et al. (2010) Genome research 20:281-290; Parks et al. (2013) Cell Metab 17:141-152). Clinical traits were assessed using 8-12 mice per strain, whereas gene expression profiling was performed using 3 mice per strain per tissue.

Statistics

Unless otherwise specified, statistical analyses were performed with Student's t-test (two groups) or one-way ANOVA (>two groups) where appropriate. Data are calculated as the mean±SEM (standard error of the mean) unless otherwise indicated. Statistical significance is represented in figures by *, p≤0.05; **, p≤0.01 unless otherwise noted.

### Example 4: Targeting Catabolic Defect of Branched-Chain Amino Acid to Treat Insulin Resistance

In the current study, the dietary intervention and pharmaceutical approaches targeting BCAA catabolic defect were explored to reveal the causal role of BCAA catabolic defect in IR in obese mice and identify the potential treatments for diabetes. It was found that reducing BCAA intake from diet efficiently improved glucose tolerance and insulin sensitivity in obese mice. Chemical inhibitor of BCKDK restored BCAA catabolism and markedly attenuated IR in both a genetic and diet-induced obese mouse models. Thus targeted manipulation of BCAA metabolism may serve as new strategy to treat insulin resistance.

### BCAA Catabolic Defect in Obese Mice is Characterized by BCKD Deficiency

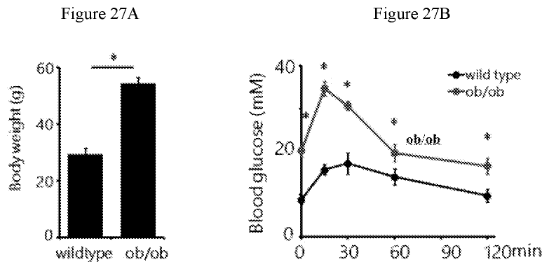

A targeted metabolomics analysis was performed to profile the plasma BCAA and their metabolites in 10 week old ob/ob mice when clinic features of IR had developed (FIG. 27 and FIG. 21A). Although plasma levels of fasting BCAA were unchanged comparing to wild type mice, the plasma BCKA concentrations were significantly elevated (FIG. 21B). In addition, the concentrations of BCKD downstream metabolites (isovalerylcarnitine from leucine, 2-methylbutyrylcarnitine from isoleucine, and isobutyrylcarnitine from valine) were all significantly decreased in the plasma of ob/ob mice. These changes are consistent with a deficient BCKD activity in the obese mice.

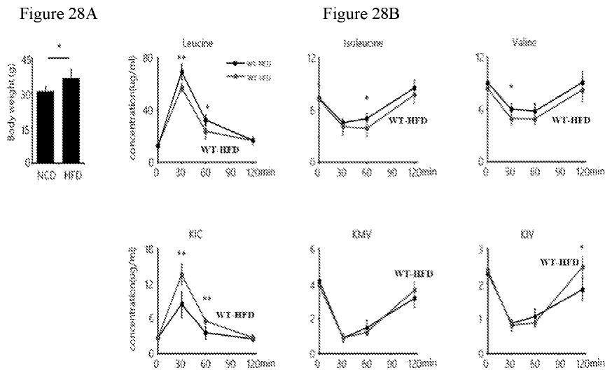

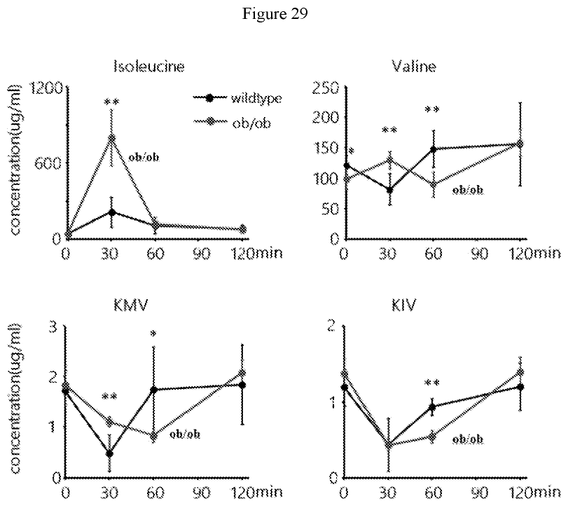

To better characterize the BCAA catabolic activity in intact animals, a BCAA tolerance test (BTT) was developed in which fasting mice were challenged with an i.p. administration of leucine followed by serial measurements of plasma BCAA/BCKA levels within two hours period. As expected, in control mice, leucine administration transiently increased the plasma leucine and KIC levels at 30 minutes post injection, which then recovered to near the basal level at 1 hour time point (FIG. 21C). In contrast, leucine administration in ob/ob mice led to significantly higher peak plasma levels of leucine and KIC and prolonged recovery to basal (FIG. 21C). Similar pattern of BCAA intolerance was also observed in high-fat-diet (HFD)-induced obese mice (FIG. 28). The magnitudes of transient induction of plasma BCKA concentrations in the obese mice were far more pronounced than those of BCAA. As expected, the plasma levels of isoleucine and valine and their corresponding keto acids were not consistently elevated by leucine challenge (FIG. 28 and FIG. 29). These data clearly demonstrate BCAA tolerance defects in obese mice, as would be expected for BCKD activity deficient mice (Lu et al. (2009), supra).

To reveal the tissue specific contribution to the systemic BCAA catabolic defect in obese mice, the metabolomics profiles of liver, white adipose tissue, and skeletal muscle were analyzed under fasting conditions (FIG. 21D-F). White adipose tissue demonstrated elevated BCAA and BCKA levels in combination with markedly lower levels of downstream metabolites in the obese mice relative to lean mice. In contrast, both BCAA and downstream metabolites showed lower abundance in liver while no significant changes were observed for BCAA and downstream metabolites in skeletal muscle. BCKA were below detection limits in liver and skeletal muscle by metabolic profiling. These data indicate that impaired BCAA catabolism in white adipose tissue and liver has potential major contribution to systemic BCAA catabolic defects observed in obese mice.

To further investigate tissue specific contribution to BCAA catabolic defects, BCAA catabolic gene expression among different tissues was measured (FIG. 21G). In white adipose tissue, BCKDE1α, BCKDE2, and PP2Cm were significantly downregulated in obese animals compared to lean controls. In liver, BCKDE2 and PP2Cm were downregulated, accompanied with a dramatic increase of BCKD E1α phosphorylation consistent with inhibited BCKD activity. In skeletal muscle, no significant change of BCKD subunits was detected.

Together, these data clearly demonstrate a systemic defect in BCAA catabolism in obese mice, characterized by impaired BCAA tolerance, deficient BCKD activity in liver and white adipose tissue, elevated BCAA/BCKA levels, and reduced abundance of BCKD metabolites.

### BCAA Catabolic Defect Promotes Insulin Resistance in Obese Mice

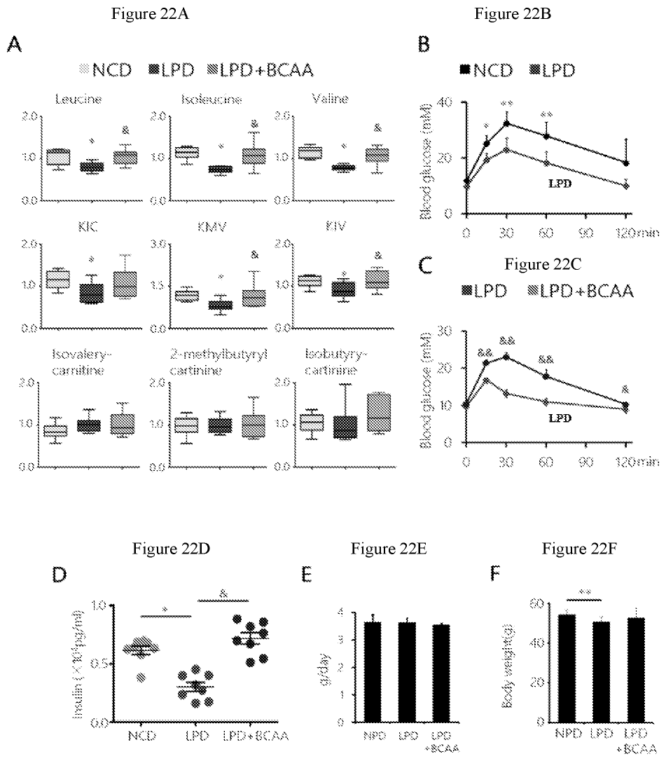

To demonstrate BCAA homeostasis has a direct causal role in the onset of insulin resistance, BCAA uptake in ob/ob mice was reduced by feeding the animal an isocaloric low protein diet (LPD, 6% protein by weight vs. normal diet NCD, 20% protein by weight). As shown in FIG. 22A, LPD significantly reduced the plasma levels of BCAA/BCKA as expected (FIG. 22A), and markedly improved glucose tolerance (FIG. 22B) in ob/ob mice. In contrast, increasing dietary intake by administrating BCAA in water raised plasma levels of BCAA/BCKA but not downstream metabolites (FIG. 22A), and significantly worsened glucose tolerance of the ob/ob mice on low protein diet (FIG. 22C). As expected, the hyperinsulinemia in ob/ob mice was significantly improved by LPD treatment but reversed by supplement of dietary BCAA (FIG. 22D). The body weight of ob/ob mice was slightly reduced by LPD treatment, however, BCAA supplement did not affect the food intake or body weight (FIG. 22E-22F). These results illustrate a direct causal role of BCAA catabolic defect and BCAA intake in the onset of insulin resistance.

Increasing BCAA Intake Impairs Insulin Sensitivity Independent of mTOR Activation and Plasma BCAA

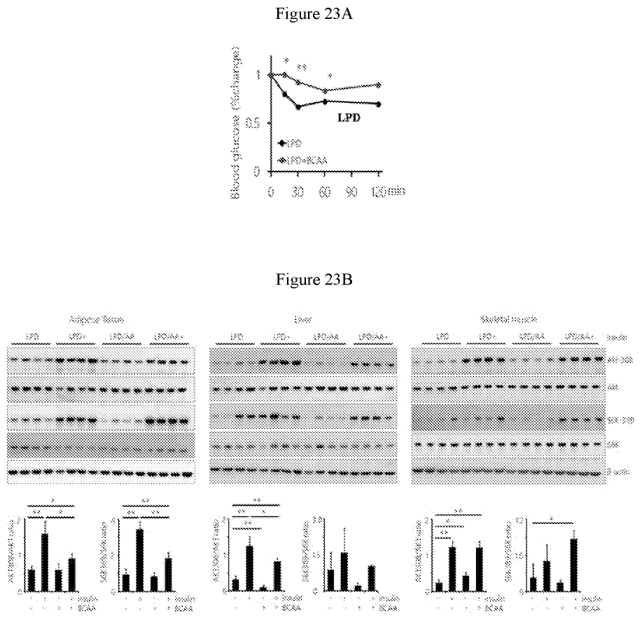

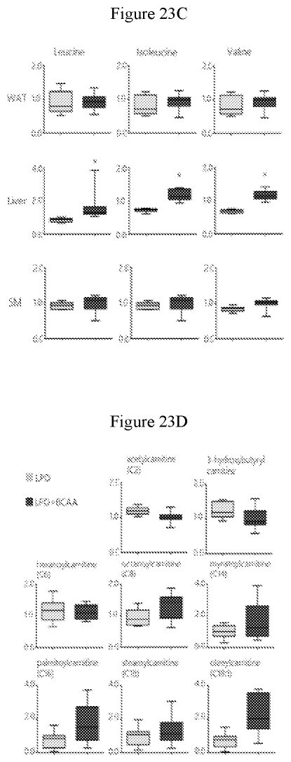

Based on insulin tolerance test (ITT), increasing BCAA significantly impaired systemic insulin sensitivity in ob/ob mice (FIG. 23A). Further examination of insulin signaling in fat, liver and skeletal muscle revealed an impaired insulin response (indicated by insulin stimulated induction of pAKT-Thr308) in white adipose tissue and liver, but not in skeletal muscle after increasing BCAA intake (FIG. 23B). It is noted, however, impaired insulin signaling was not associated with altered steady state mTOR activity (FIG. 23B). In addition, metabolomics analysis revealed elevated intra-tissue levels of BCAA in liver following increasing BCAA uptake, but not in skeletal muscle and white adipose tissue (FIG. 23C). Therefore, the abundance of intra-tissue BCAA levels were not consistently correlated with insulin response nor with intra-tissue mTOR activity. A recent report has suggested that enhanced BCAA oxidation interferes with complete lipid oxidation in skeletal muscle, leading to the accumulation of lipid metabolic intermediates that promotes IR (White et al. (2016) Molecular Metabolism 5:538-551). The present metabolomics analysis also detected a trend of elevated level of long chain acylcarnitines in the skeletal muscle in mice with increased BCAA intake (FIG. 23D). But this subtle accumulation was not associated with impaired insulin sensitivity in skeletal muscle (FIG. 23B). Together, these data suggest that increasing BCAA intake impairs insulin signaling in a tissue-specific manner, most significantly observed in liver and white adipose tissue but not in skeletal muscle, and the insulin signaling defect was independent of mTOR activity or intra-tissue abundance of BCAA.

Adipose BCAA Catabolic Pathway is Specifically Associated with Insulin Resistance in Human and Mouse Populations

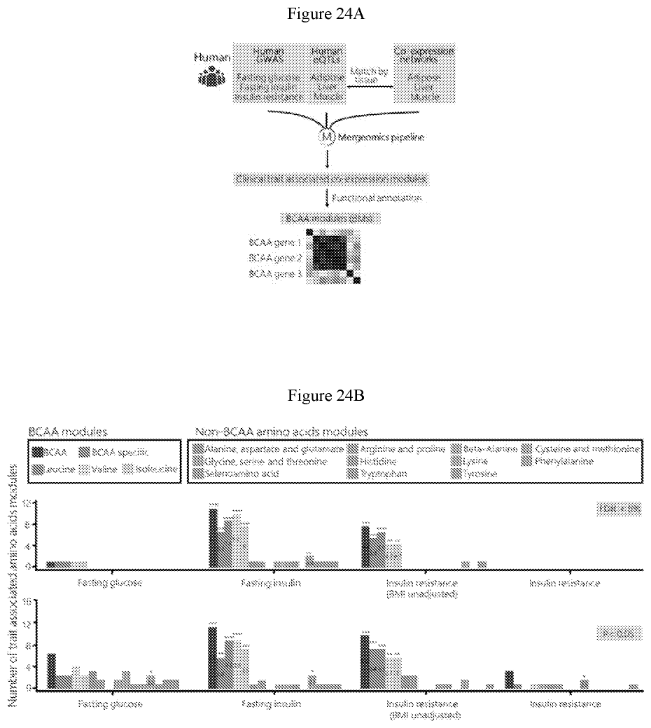

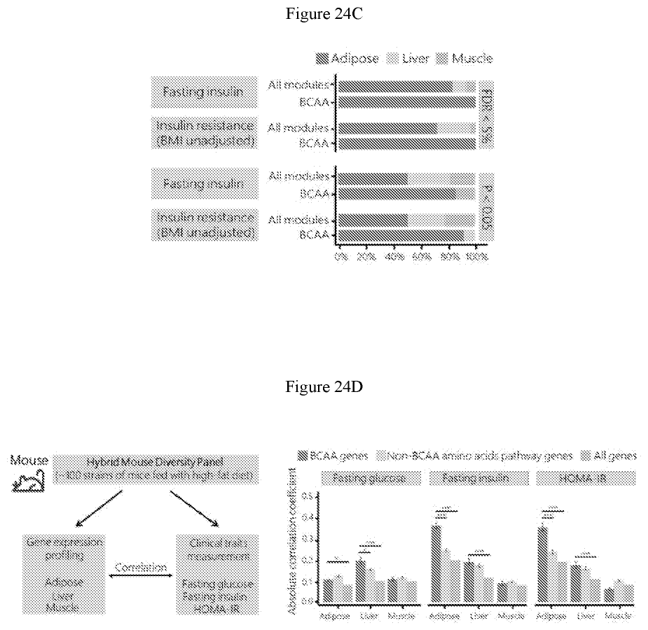

To evaluate the significance of the functional connection between BCAA catabolic pathway and insulin resistance as we established in the ob/ob mice, existing large-scale genetics and functional genomics datasets from human and mouse populations were leveraged using an integrative framework (FIG. 24A).

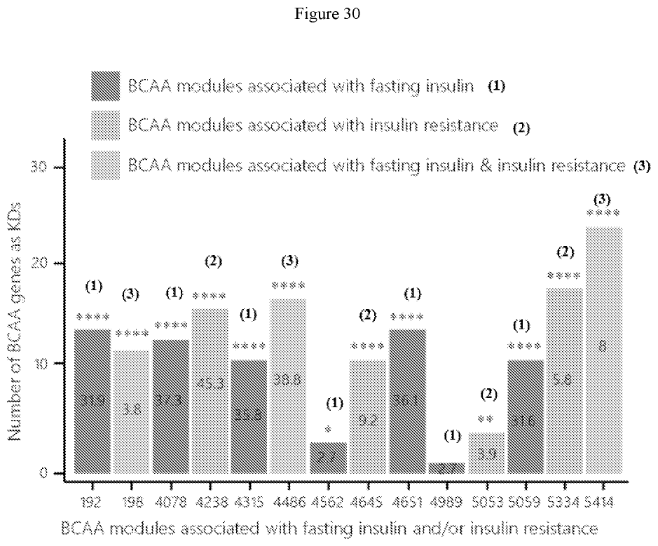

For the human focused analysis, genome-wide association studies (GWAS) of IR and related glycemic traits were compiled from the GENESIS and MAGIC consortia, and tissue-specific expression quantitative trait loci (eQTLs) and co-expression network modules containing sets of co-regulated genes in adipose, liver, and muscle tissues (FIG. 24A, see Methods in Example 3). These diverse datasets were integrated using the Marker Set Enrichment Analysis (MSEA) implemented in our Mergeomics pipeline (Shu et al. (2016), supra) to identify co-expression modules whose gene members show significant enrichment of genetic association with human IR and related metabolic traits. To differentiate the roles of white adipose tissue, liver, and skeletal muscle, the tissue origin of co-expression modules and eQTLs was matched when mapping GWAS SNPs to genes in the co-expression modules. Among all co-expression modules that demonstrated significant genetic association with fasting insulin and IR traits at either false discovery rate (FDR)<5% correcting for multiple testing or uncorrected p<0.05, significant over-representation of BCAA modules was observed (FIG. 24B). In contrast, BCAA modules were not enriched among co-expression modules associated with fasting glucose or body mass index (BMI)-adjusted IR, suggesting an adiposity-dependent association between BCAA and IR (FIG. 24B). Additionally, no association was observed for non-BCAA amino acid pathways (FIG. 24B), supporting a specific role of BCAA metabolism in human IR. When evaluating the tissue origin of co-expression modules, the BCAA modules associated with fasting insulin and BMI-unadjusted IR were found to be primarily from the adipose tissue, compared to the more heterogeneous tissue distribution among all significant modules (FIG. 24C). In addition, the weighted Key Driver Analysis (wKDA) was employed in the Mergeomics pipeline to pinpoint central network genes, termed key drivers, which orchestrate or regulate the genes in the 14 significant BCAA associated modules (7 associated with fasting insulin, 4 associated with IR and 3 associated with both traits) at FDR<5% (see Methods in Example 3). It was found that BCAA catabolic genes were among the key drivers for all 14 BCAA associated modules, supporting that BCAA metabolic genes play pivotal regulatory role in the IR-associated BCAA modules (FIG. 30).

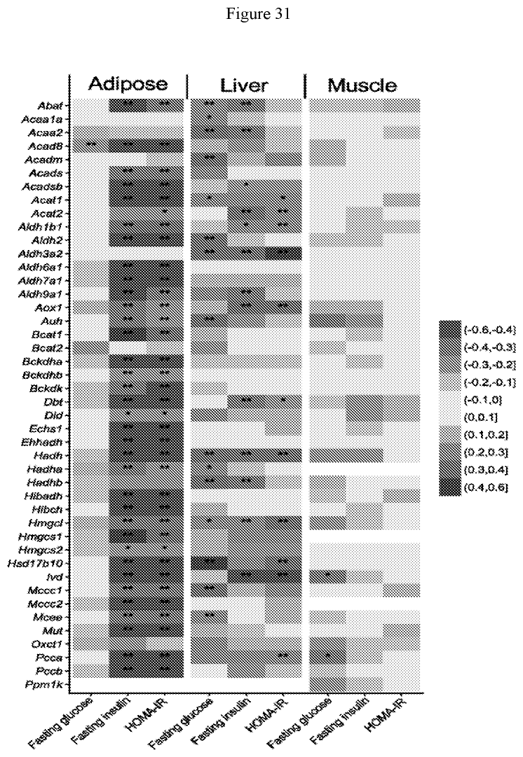

For the mouse-specific analysis, the association of the expression levels of individual BCAA genes in adipose, liver and muscle with IR-relevant traits we evaluated in ˜100 genetically divergent mouse strains in the Hybrid Mouse Diversity Panel (HMDP) fed with a high-fat diet (FIG. 24D) (Bennett et al. (2010), supra; Parks et al. (2013), supra). Consistent with the findings from the above human studies and the ob/ob mice, the expression levels of BCAA metabolic genes in the adipose tissue exhibited strong negative correlation with fasting insulin level and IR (HOMA-IR) in the HMDP cohort, but not fasting glucose level (FIG. 24D, FIG. 31). Although BCAA gene expression levels in the liver also showed a mild degree of association with fasting insulin and IR traits (FIG. 31), the correlation strength was not significantly different from the genes in non-BCAA amino acid pathways (FIG. 24D). The muscle tissue, on the other hand, did not exhibit specific correlations between BCAA and IR or other glycemic traits (FIG. 24D).

In summary, consistent with the findings from the ob/ob mouse model, unbiased multi-scale systems analysis from both human and mouse populations revealed robust genetic evidence that supports a unique and strong association between IR and BCAA metabolic pathway, particularly in adipose tissue.

### Adipokine Sfrp5 Mediates the Effect of BCAA Catabolic Defect on Insulin Sensitivity

To explore the underlying mechanisms linking BCAA catabolic defects to insulin resistance, whole transcriptome profiling was performed in white adipose tissue since it is a major contributor based on tissue specific analysis in ob/ob mice and systems studies from human and mouse populations. By differential gene expression analysis between wild type and PP2Cm-ablated mice where BCAA catabolism is defective and BCAA/BCKA accumulate (Lu et al. (2009), supra), a reduced expression of secreted frizzled-related protein 5 (Sfrp5) was found in the PP2Cm-ablated adipose tissue. Sfrp5 is an adipokine with insulin-sensitizing and anti-inflammatory properties and its expression is reduced in animals with obesity and metabolic disorder (Ouchi et al. (2010) Science 329:454-457). Lowering circulating BCAA by reducing protein intake in PP2Cm-null mice effectively restored Sfrp5 expression in the white adipose tissue while increasing protein intake in wild type mice significantly down-regulated Sfrp5 gene expression in the white adipose tissue (FIG. 25A). In cultured differentiated 3T3-L1 adipocytes, BCKA treatment potently suppressed Sfrp5 expression while BCAA demonstrated a similar trend, showing a direct cell-autonomous effect of BCKA/BCAA on Sfrp5 suppression (FIG. 25B). In ob/ob mice, low protein diet enhanced Sfrp5 expression while increasing BCAA intake again suppressed it in white adipose tissue (FIG. 25C), concurrently with the BCAA's influence on IR (FIG. 22B-22D and FIG. 23A). Importantly, ectopic expression of Sfrp5 via systemic delivery of a Sfrp5 expressing adenovirus restored glucose tolerance (FIG. 25D-25E), reduced plasma insulin and TNFα level in the obese/IR mice with high BCAA intake, (FIGS. 25F and 25G). These data suggest loss of Sfrp5 expression in adipose tissue is at least in part responsible for the impact of BCAA catabolic defect on systemic IR in ob/ob mice.

### Chemical Inhibitor of BCKDK Corrects BCAA Catabolic Defect and Attenuates Insulin Resistance in Obese Mice

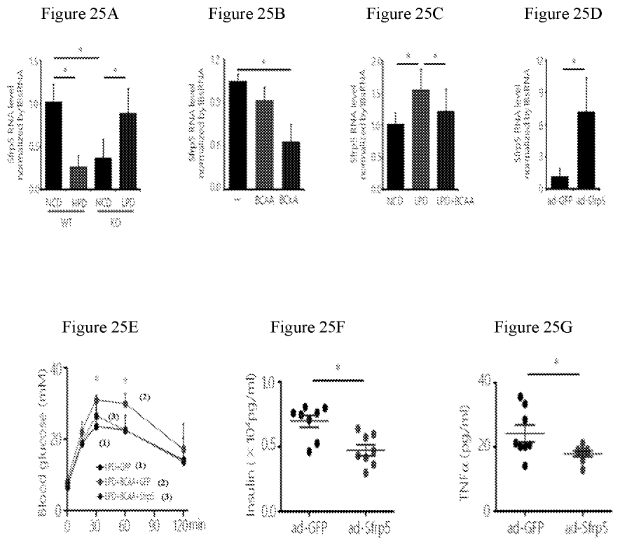

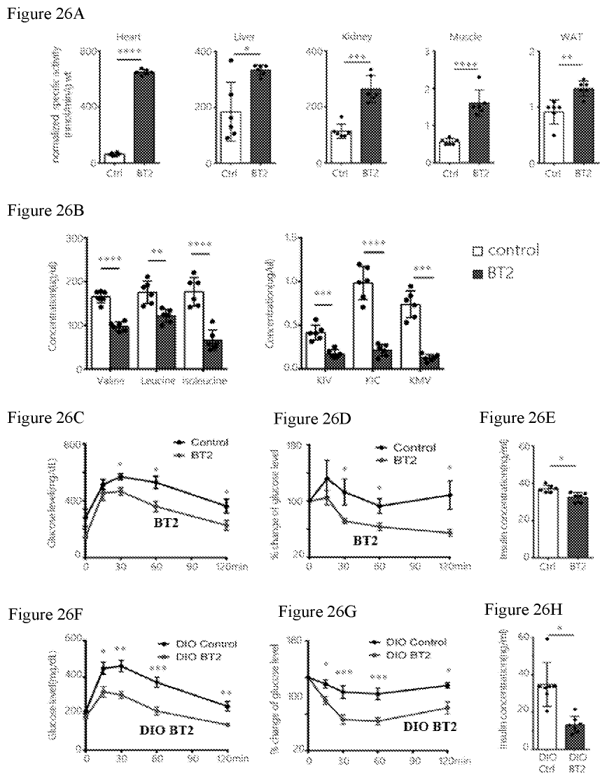

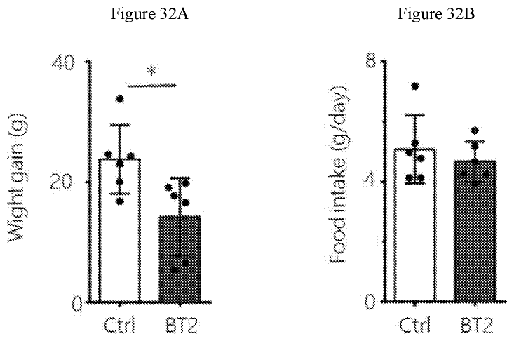

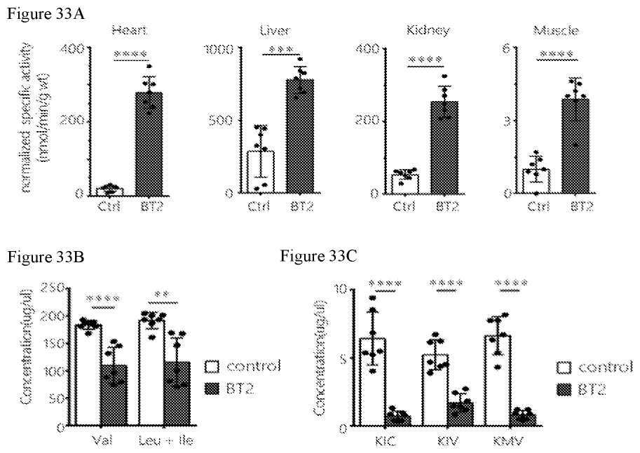

Given the significant contribution of BCAA catabolic defect to IR, the therapeutic impact of restoring BCAA catabolic activity on glycemic control was investigated in obese mice. Administration of BT2 (3,6-dichlorobenzo[b]thiophene-2-carboxylic acid), a specific and potent inhibitor of BCKDK (Tso et al. (2014), supra), significantly enhanced BCKD activity in different tissues of ob/ob mice, including white adipose tissues (FIG. 26A). Plasma BCAA levels in the ob/ob obese mice were significantly lowered following BT2 treatment; whereas the magnitudes of reductions in plasma BCKA concentrations were far more pronounced (FIG. 26B). Importantly, BT2-treated mice displayed significantly improved glucose tolerance and insulin sensitivity (FIGS. 26C and 26D), accompanied by the attenuation of hyperinsulinemia (FIG. 26E). BT2 treatment did not affect food intake but slightly reduced body weight gain of the ob/ob mice (FIG. 32). In parallel experiments, BT2 treatment also augments BCKD activity in heart, muscle liver and kidney and lowers plasma BCAA/BCKA levels in DIO mice (FIG. 33). Like in ob/ob mice, BT2 treatment also improved glucose tolerance, insulin sensitivity, and hyperinsulinemia in high fat diet (HFD)-induced obese (DIO) mice (FIG. 26F-26H). These results demonstrate that correcting BCAA catabolic defect by targeted inhibition of BCKDK with a small molecule inhibitor is a potentially effective therapeutic approach to treat IR in obese and Type 2 diabetes.

In the present study, it was demonstrated that in obese mice, BCAA catabolic defect in white adipose tissue due to BCKD deficiency was manifested in an accumulation of BCAA/BCKA and loss of systemic BCAA/BCKA tolerance. Restoration of BCAA homeostasis in obese mice by lowering BCAA intake effectively improved insulin sensitivity while increasing BCAA intake promoted IR, demonstrating a causal role of BCAA in the pathogenesis of IR. Further integrative genomic analyses of both human and mouse populations with diverse genetic background revealed a specific and robust association of IR with BCAA catabolic pathway, particularly in adipose tissue, substantiating the findings from the obese mouse model as a potential common mechanism in diabetes. At mechanistic level, instead of mTOR activity, a direct inhibitory effect of BCKA on Sfrp5 in adipose tissue was found to contribute to BCAA mediated regulated IR. Importantly, enhancing BCAA catabolic activity by a small molecule BCKD kinase inhibitor effectively attenuated IR in obese mice. All of these findings suggested that BCAA catabolic defect is a significant contributor to the pathogenesis of insulin resistance, and BCAA catabolic pathway is a valid and potentially important target of pharmacological intervention to improve insulin signaling in diabetes.

An unexpected observation in this study was the lack of significant association between elevated fasting plasma BCAA and IR. High level of BCAA in blood has been reported as a metabolic hallmark of IR and diabetes in numerous studies (Lynch and Adams (2014), supra; Lu et al. (2013) Front. Med. 7:53-59; Lu et al. (2016) Diabetologia 59:2349-2359; Chen et al. (2016) Sci Rep 6:20594), although there was also evidence showing lower plasma BCAA level in diabetic subjects. On the other side, some recent studies noted an elevated BCKA level in blood in obese animals, associated with impaired fasting glucose and diabetes (White et al. (2016), supra; She et al. (2013) PLoS ONE 8:e59443; Menni et al. (2013) Diabetes 62(12):4270-4276). In the current study, it was found the plasma level of BCKA was elevated and tightly associated with the glycemic effect of BCAA intake (FIG. 22A). In addition, it was demonstrated that BCAA tolerance defects were evident and highly associated with the state of insulin resistance in mice. Therefore, plasma BCKA or leucine tolerance test as depicted in this study would be a better biomarker and more accurate diagnostic approach to detect BCAA catabolic deficiency leading to insulin resistance.

Although BCAA, particularly leucine, is a potent signal molecule to activate mTOR, and BCAA-stimulated mTOR over-activation has been implicated to impair insulin signaling in cells (Newgard et al. (2009) Cell Metabolism 9:311-326), the present metabolomic and molecular characterization showed that intra-tissue level of BCAA was not well correlated with either steady state mTOR activity or insulin signaling in vivo. Although a potential effect on the dynamic regulation of mTOR signaling cannot be excluded, it appears that intra-tissue BCAA level is not the only determinant to mTOR activity or insulin signaling.

The importance of adipose tissue in maintaining BCAA and lipid homeostasis and systemic insulin sensitivity is supported by tissue-specific manifestation of BCAA catabolic defects in the ob/ob mouse model and unbiased integrative genomics analyses from multi-scale human and mouse public datasets. At mechanistic level, elevated BCAA/BCKA exposure significantly reduced the expression of Sfrp5 in vitro and in vivo, which was demonstrated as a potential mediator linking BCAA/BCKA accumulation in adipose tissue with systemic insulin resistance (Newgard et al. (2009), supra; Ouchi et al. (2010), supra). Although some reports have argued against the “insulin sensitizing” and “anti-inflammatory” function of Sfrp5 (Mori et al. (2012) The Journal of Clinical Investigation 122:2405-2416; Wang et al. (2014) Journal of Molecular Endocrinology 53:405-415), the present data showed restoring plasma Sfrp5 through ectopic expression reduced plasma level of TNFα and alleviates BCAA-overload induced IR in ob/ob mice.

Giving the importance of BCAA catabolic defects in the pathogenesis of insulin resistance, the therapeutic potential of targeted manipulation of BCAA catabolic activity through small molecule treatment had not yet been demonstrated. In both ob/ob and DIO obese mice, treatment with the BCKDK inhibitor BT2 results in robust increases of BCKD activity in major tissues tested (heart, kidney, muscle, and liver). The marked enhancement of BCKD activity led to drastically lower BCAA/BCKA concentrations, leading to improved glucose tolerance and insulin sensitivity in obese animals. This is the first in vivo proof-of-concept demonstration that validates targeting BCAA catabolic pathway as a potentially important therapeutic approach to improve insulin sensitivity and to treat diabetes. More pre-clinic studies in other clinically relevant type 2 diabetes models will follow.

Obesity and related comorbidities are major public health threats worldwide. It is well documented that dietary modification can exert beneficial effect to reduce the progression of IR and diabetes. High protein intake has been considered beneficial for certain metabolic parameters, but whether high-protein diet improves glycemic control in obese patients is still very controversial (Ajala et al. (2013) The American Journal of Clinical Nutrition 97:505-516; Khazrai et al. (2014) Diabetes/Metabolism Research and Reviews 30:24-33). The present study identifies a casual role of BCAA catabolic defect in IR in obese animals, and offers a compelling mechanistic basis for the strong association between elevated plasma BCAA/BCKA and the onset of IR in obesity. These results highlight the potential impact of protein/BCAA intake on promoting IR in obese people. Therefore, nutrition guidelines for diabetes management may need to be re-evaluated with the consideration of adiposity level and systemic BCAA catabolic activities. Plasma BCKA and BCAA (Leucin) tolerance test (LTT) may be used as valuable biomarker and effective diagnostic tool to stratify insulin resistant patients from general at risk population for dietary modulation and BCKDK inhibitor therapy.

### Example 5: Efficacy of BCKDK Inhibition on Disease Progress in Heart Failure

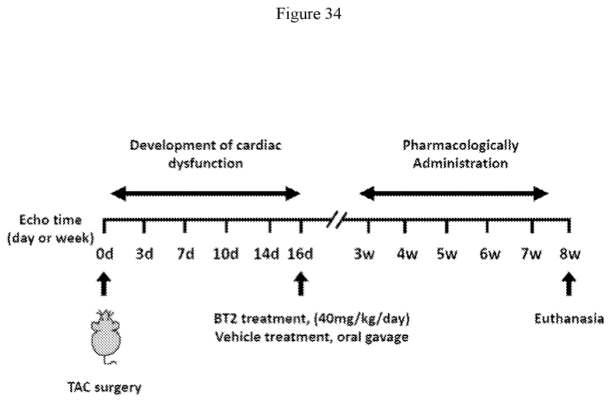

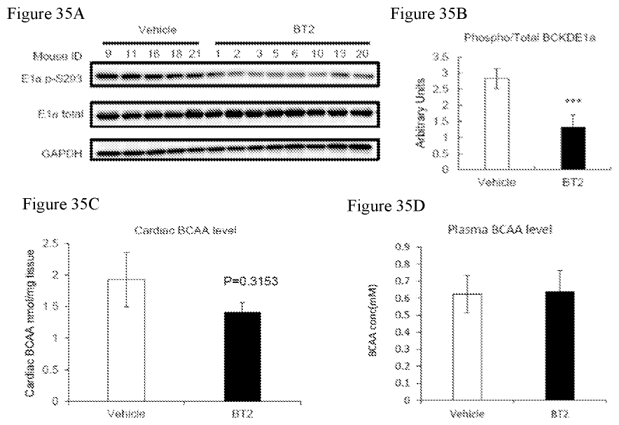

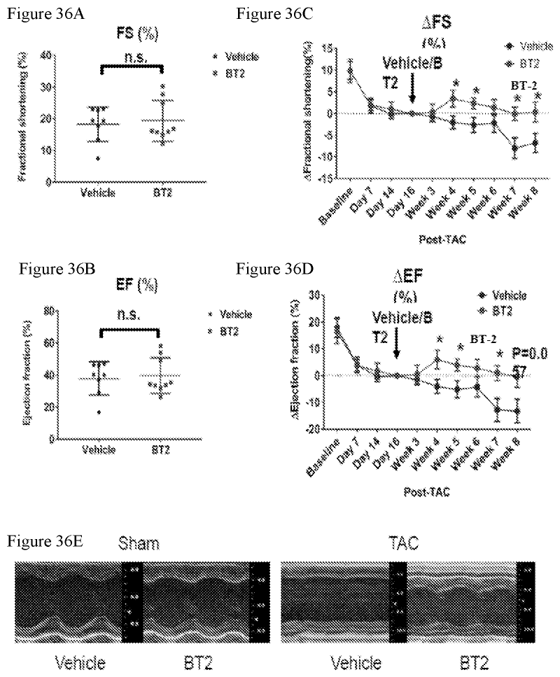

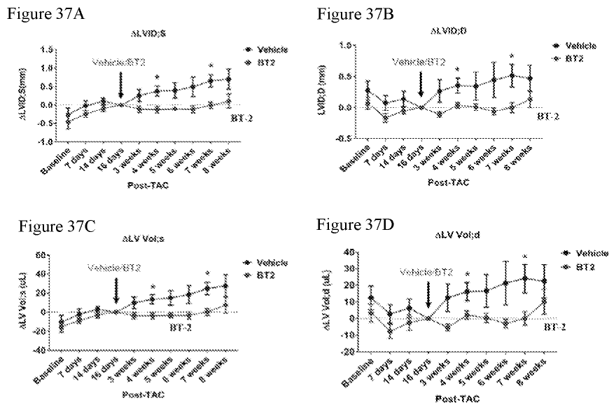

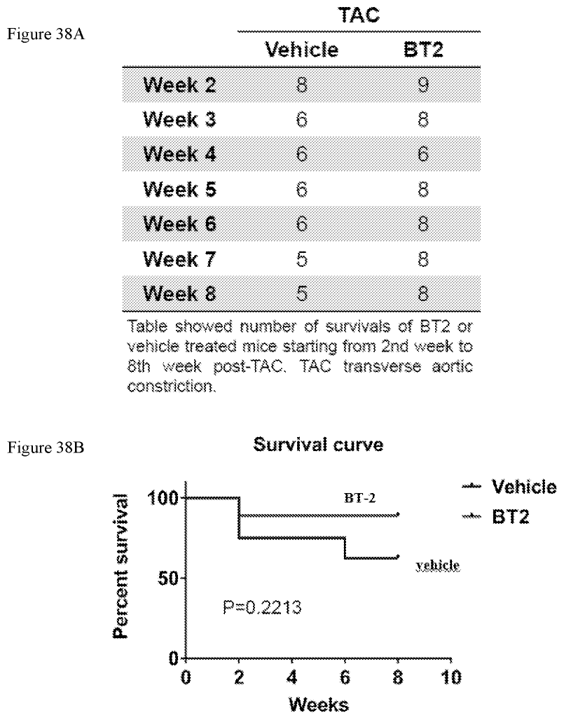

BT-2 treatment was carried out in a mouse model of heart failure. Specifically, mice were intragastrically administered with BT-2 or vehicle for six weeks and cardiac function was assessed every week (FIG. 34). As the result, BT-2 enhanced cardiac BCAA catabolic activities post-TAC (FIG. 35). Systolic function was preserved in pressure-overloaded mouse heart (FIG. 36). Clearly, BT-2 therapy alleviated pressure-overload-induced cardiac structural remodeling (FIG. 37) and improved mouse survival (from two weeks after treatment as the first time point of sampling) (FIG. 38).

### Example 6: Exemplary Materials and Methods Used in Example 7

Animals

Wild type C57BL/6J mice were purchased from ENVIGO, and housed at 22° C. with a 12-hour light, 12-hour dark cycle with free access to water and standard chow. Studies were performed with male mice. Terminal tissue collection was performed on mice under isoflurane anesthesia with additional cervical dislocation. All animal procedures were carried out in accordance with the guidelines and protocols approved by the University of California at Los Angeles Institutional Animal Care and Use Committee (IACUC).

Transverse Aortic Constriction

In mice, transverse aortic constriction (TAC) was performed as described (Lee et al. (2011) Circulation Research 109:1332-1341) in anesthetized (pentobarbital 60 mg/kg, IP) and ventilated mice (age 12-14 weeks) to induce hypertrophy and heart failure. After left anterolateral thoracotomy with blunt dissection through the intercostal muscles, aortic constriction was induced by ligating the transverse aorta around a 27½-gauge blunt needle using 6-0 silk suture. The needle was subsequently removed. Sham-operated mice underwent a similar surgical procedure without constriction of the aorta. All mice were maintained in the same environment with regular lab chow and water ad libitum. At the end of the experiments, animals were euthanized and the hearts and lungs were removed and weighed. Hearts were dissected and tissues were either immediately immersed into 4% buffered formaldehyde or quickly frozen in liquid nitrogen for further experiments.

BT2 Treatment

Compound BT2 (3,6-dichlorobenzo[b]thiophene-2-carboxylic acid) was a kind gift from doctor David T. Chuang lab (University of Texas Southwestern Medical Center). Administration of BT2 was performed as previously described (Tso et al. (2014) Journal of Biological Chemistry 289:20583-20593) except that animals (age˜12 weeks) were dosed daily by oral gavage at 40 mg/kg/day. Administration of BT2 started 2 weeks after TAC surgery and continued for 6 weeks post-TAC.

Echocardiography

The mice were anesthetized and maintained with 1-2% isofluorane in 95% oxygen. Echocardiography was performed with a VisualSonics Vevo 770 (VisualSonics Inc, Toronto, Canada) equipped with a 30-MHz linear transducer. A parasternal short axis view was used to obtain M-mode images for analysis of fractional shortening, ejection fraction, and other cardiac parameters. Speckle-tracking echocardiography was performed as described previously (Bhan et al. (2014) American Journal of Physiology—Heart and Circulatory Physiology 306:H1371-H1383). Strain analyses were conducted by the same trained investigator on all animals and using a speckle-tracking algorithm provided by VisualSonics (VevoStrain, VisualSonics). In brief, suitable B-mode loops were selected from digitally acquired echocardiographic images based on adequate visualization of the endocardial border and absence of image artifacts. Three consecutive cardiac cycles were selected for analysis based on image quality. Semi-automated tracing of the endocardial borders were performed and verified over all 3 cardiac cycles and then corrected as needed to achieve good quality tracking throughout each cine loop. Tracked images were then processed in a frame-by-frame manner for strain measurements. Strain measures were averaged over the obtained cardiac cycles (with temporal smoothing filters turned off for all measurements), resulting in curvilinear strain and SR data. Each long- and short-axis view of the LV myocardium was divided into 6 standard anatomic segments for regional or global speckle-tracking based strain analysis throughout the cardiac cycle. For global strain values, peak strain and SR measurements were averaged across all 6 segments, as well as the motion measurements (velocity and displacement). Parasternal long-axis views were found to provide the most reproducible myocardial views for longitudinal strain analyses in mice, whereas parasternal short-axis views (at the mid-papillary level) were obtained for circumferential and radial (short-axis) strain analyses. For the diastolic myocardial strain analysis, the data presented in this study are the mean global strain rate values at all three axes: longitudinal, radial and circumferential. As described in previous study (Schnelle et al. (2017) Journal of Molecular and Cellular Cardiology 114:20-28), to quantify the peak strain rate during early LV filling, the “reverse peak” option in the Vevo2100 1.5.0 image Software was used, and obtain this parameters of peak reverse strain rate at different planes (longitudinal, radial and circumferential).

Western Blot Analysis

Proteins from heart tissue were harvested in buffer (50 mM HEPES [pH7.4], 150 mM NaCl, 1% NP-40, 1 mM EDTA, 1 mM EGTA, 1 mM glycerophosphate, 2.5 mM sodium pyrophosphate 1 mM Na3VO4, 20 mM NaF, 1 mM phenylmethylsulfonyl fluoride, 1 μg/mL of aprotinin, leupeptin, and pepstatin). Samples were separated on 4-12% Bis-Tris gels (Invitrogen), and transferred onto a nitrocellulose blot (Amersham). The blot was probed with the indicated primary antibodies. Protein signals were detected using HRP conjugated secondary antibodies and enhanced chemiluminescence (ECL) western blotting detection regents (Pierce). Rabbit polyclonal antisera against the E1α subunits of BCKD complex and phosphor-E1α antibodies were purchased from Abcam. PP2Cm antibody was generated in the lab.

Real-Time RT-PCT Analysis

Total RNA was extracted from heart tissues using Trizol Reagent (Invitrogen) according to the manufacturer's instructions. Total RNA was reverse-transcribed into the first-strand cDNA using ProtoScript® II Reverse Transcriptase (New England BioLabs). Then, cDNA transcripts were quantified by the CFX Real-Time PCR System (BioRad). 18sRNA were used for normalization except where indicated. PCR primer information is available upon request.

Cardiac BCAA and BCKA Assay

BCAA and BCKA concentration in mouse heart tissue were determined by mass spectrometer using the following transition s: KIC 203.1 to 161.1 (retention time: 2.91 min); KIV 189.145 to 119.2 (retention time 2.84 min); KMV 203.065 to 174.2 (retention time 2.99 min); [¹³C] KIV 194.109 to 120.1 (retention time: 2.84 min). Val 174.1/72.1 (retention time 3.2 min), Leu 188.1/86.1 (retention time 5.9 min), Ile 188.2/86.1 (retention time 5.7 min).

Statistics Analysis

All the data were presented as mean±standard error of mean (SEM) and were analyzed using Graphpad Prism software (Graphpad Software, USA). For two groups, the student's t-test was conducted. P values less than 0.05 were considered as statistically significant.

### Example 7: Therapeutic Efficacy of Pharmacological Inhibition of BCKDK

In the current study, the therapeutic efficacy of restoring BCAA metabolic flux through pharmacological inhibition of BCKDK in a mouse heart failure model induced by chronic pressure overload was assessed. Genetic and cellular analysis suggest that BCAA catabolic defect is not only a consequence but also a significant contributor to heart failure progression, at least in part due to elevated ROS and metabolic disturbance (Sun et al. (2016) Circulation 133:2038-2049) such as suppressed glucose metabolism (Li et al. (2017) Cell Metabolism 25:374-385). These findings highlight the importance of BCAA metabolism for maintaining cardiac function in response to pathological stress. Notably, two recent studies reported cardiac protective effects of BCKDK inhibitory treatment by BT2 compound in TAC and MI mouse model (Sun et al. (2016) supra; Wang et al. (2016) American Journal of Physiology—Heart and Circulatory Physiology 311:H1160-H1169) and these beneficial effects of BT2 treatment started from the onset of surgical operation before pathologic features emerging. However, in clinical practice, therapeutic intervention usually begins at the time of symptom development. Therefore, it's persuasive to ask whether the benefits from BT2 treatment would be observed following existing pathological changes and test its therapeutic potential for the treatment of HF.

Establishment of a Pharmacologically Treated Mouse Model with Preexisting Cardiac Dysfunction

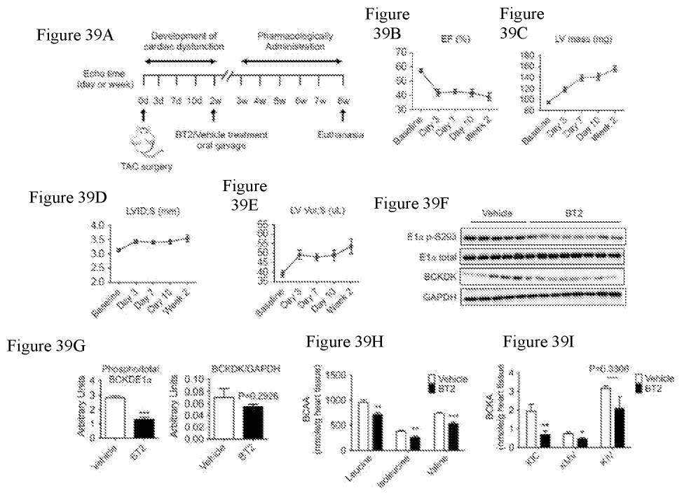

Often, patients present to medical attention at the time of symptom development. As such, prevention of existing pathological deterioration is a typical objective of therapeutic intervention. In order to investigate the functional impact of BCKDK inhibition on the progression of heart failure, pressure-overload to left ventricle was initiated by transaortic constriction (TAC) for two weeks followed by treating the animals with 40 mg/kg/day BT2 or vehicle via oral gavage for 6 additional weeks (FIG. 39A). In the hearts with aortic stricture before treatment, very robust hypertrophic growth with dramatically increased LV mass and ventricular dilatation, and significant declines in systolic function demonstrated by echocardiography were detected (FIG. 39B-FIG. 39E).

Mice were then randomly assigned to receive BT2 or vehicle for additional 6 weeks. As expected, BT2 treatment significantly reduced BCKDK mediated E1α phosphorylation versus vehicle treated samples with only insignificant effect or BCKDK protein expression, which implicated enhanced BCKDH activities (FIG. 39F and FIG. 39G). Cardiac intra-tissue concentrations of BCAA and BCKA were significant reduced (FIG. 39H and FIG. 39I), consistent with enhanced BCAA catabolic activities and degradation flux.

BCKDK Inhibitor Therapy Alleviate Pressure Overload Induced Systolic Dysfunction without Affecting Hypertrophy

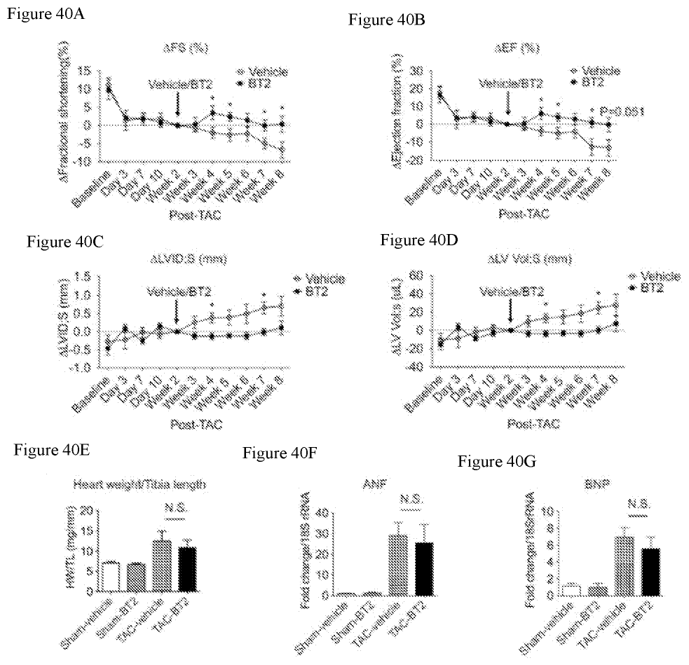

In order to detect responses in each mouse during the treatment period, changes of ejection fraction (EF) and fractional shortening (FS) were measured over time, and ΔEF and ΔFS were generated using parameters in week 2 as baseline. The average ΔEF and ΔFS in each time points were plotted (FIG. 40A and FIG. 40B). By 2 weeks of BT2 treatment (4 weeks post-TAC), an increase of FS by 3.5±1.9 points, and EF by 6.0±3.4 points from baseline in BT2 treated mouse hearts was observed, as tracked by noninvasive echocardiography. Continuation of treatment with BT2 for additional 4 weeks led to additional alleviations in systolic dysfunction, however without affecting cardiac hypertrophy indicated by heart weight and tibia length ratio (FIG. 40E) and hypertrophic markers (FIG. 40F and FIG. 40G). LV structural measurements displayed stable maintenance in the chamber size in BT2 treated mouse hearts as seen in lower changes of LV internal dimensions (ΔLVIDs, FIG. 40C) and LV volume (ΔLV Vols, FIG. 40D). In contrast, vehicle treated animals developed significant chamber dilatation, as indicated by increasing changes of LVID and LV volume during the treatment period. Together, these data demonstrated that BCKDK inhibitory therapy was capable of attenuating existing systolic dysfunction.

BCKDK Inhibitory Therapy Improved Myocardium Contractility

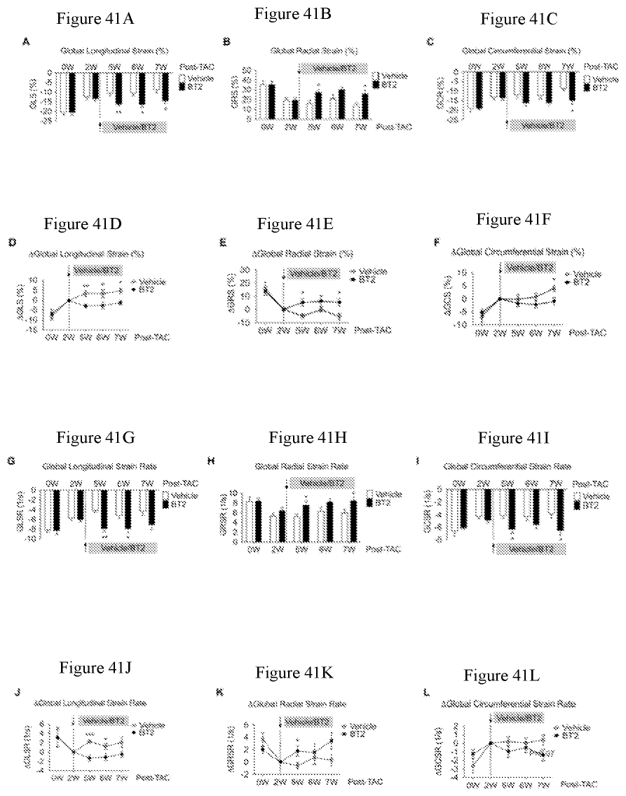

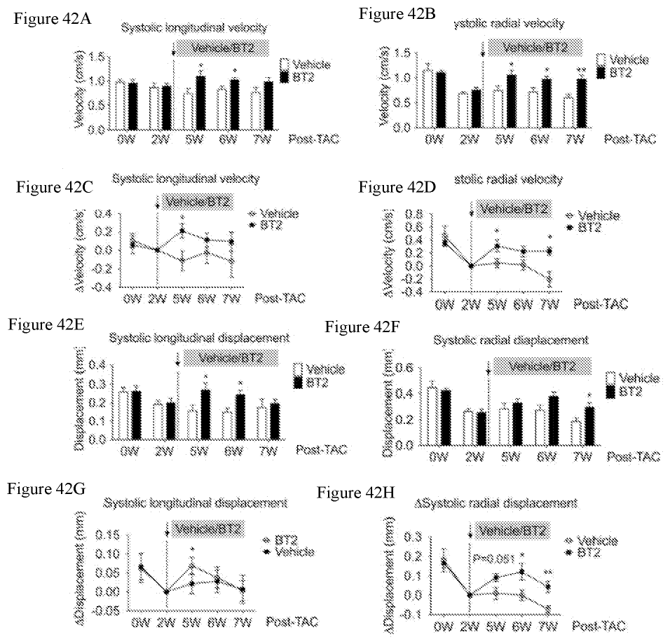

Cardiac function assessment is of great importance for the management of a broad range of heart diseases (Bijnens et al. (2009) European Journal of Echocardiography 10:216-226), however conventional echocardiographic measures such as ejection fraction (EF) and fraction shortening (FS) lack sensitivity for capturing subtle changes in LV ventricular performance (Bauer et al. (2011) Circulation Research 108:908-916) and fail to detect early myocardial dysfunction given that these parameters are typically considered late manifestations of diseases (Bauer et al. (2011), supra). Thus assessment of intrinsic cardiac function by measuring true contractility of myocardium is needed (Bijnens et al. (2009), supra). To fully describe the impact of BT2 treatment on murine myocardium contractile function in the setting of heart failure, a novel non-Doppler-based echocardiography technique was used to measure myocardial mechanics including deformation (strain and strain rate) and wall motion (velocity and displacement). With the use of Speckle Tracking Echocardiography (STE) technique, global and myocardial strain and strain rate in all three orthogonal axes (longitudinal, radial and circumferential) were assessed. Consistent with the conventional systolic measurements, BT2 treatment significantly improved myocardial strain (FIG. 41A-FIG. 41F) and strain rate (FIG. 41G-FIG. 41L) after 5 weeks post-TAC (3 weeks post-treatment). Interestingly, BT2 treatment affected longitudinal strain and strain rate most dramatically compared to radial and circumferential planes, which was plausible in that Global Longitudinal strain (GLS) has been considered as the best evaluated strain parameters (Ternacle et al. (2013) European Heart Journal—Cardiovascular Imaging 14:77-84; Smiseth et al. (2016) European Heart Journal 37:1196-1207) and has been shown as a more sensitive assessment than LVEF and a better predictor of mortality and cardiac events (Lumens et al. (2015) JACC: Cardiovascular Imaging 8:1360-1363; Adamo et al. (2017) Circulation: Heart Failure 10; Tower-Rader et al. (2017) Journal of the American Heart Association 6). Furthermore, BT2 treatment was found to increase systolic velocity and displacement in both longitudinal and radial planes (FIG. 42A-FIG. 42H), which indicated that BT2 significantly improved myocardial wall motion. Together, these data suggested that BT2 improved myocardial mechanics to preserved cardiac performance under pathological stress.

BT2 Treatment Improved Diastolic LV Mechanics of Mice with Heart Failure

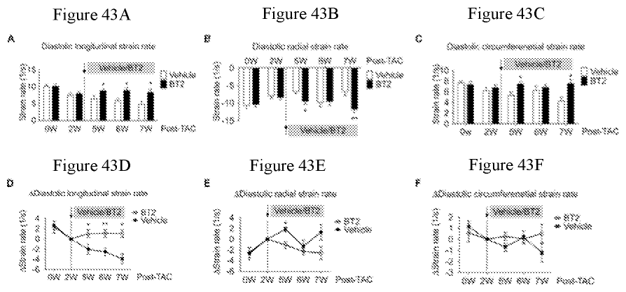

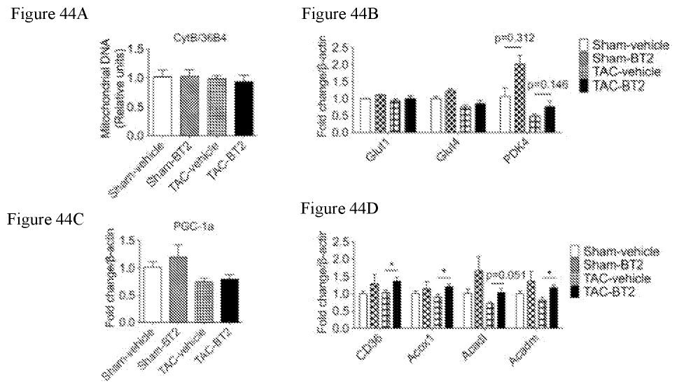

Several previous investigations suggested that early diastolic strain rate (SRe) can served as a novel marker of elevated LV filling pressure (Wang et al. (2007) Circulation 115:1376-1383; Ersboll et al. (2014) European Heart Journal 35:648-656; Flachskampf et al. (2015) JACC: Cardiovascular Imaging 8:1071-1093) and contributes to evaluation of diastolic function (Schnelle et al. (2017), supra) and myocardial viability (Park et al. (2006) American Journal of Physiology—Heart and Circulatory Physiology 290:H724-H731; Park et al. (2009) Journal of the American Society of Echocardiography 22:183-189). Based on this findings, the effect BT2 has on diastolic myocardial mechanics in mouse heart was explored by measuring peak strain rate during early LV filling through two-dimensional (2D) speckle tracking. BT2 treatment remarkably improved longitudinal SRe in mouse heart 5 weeks post-TAC (FIG. 43A), however this beneficial effect showed dispersity at radial circumferential planes during the treatment period (FIG. 43B and FIG. 43C). This finding was probably accounted for by the LV geometric changes as longitudinal deformation was well related to subendocardial myofiber dysfunction that tended to occur early in the setting of hypoperfusions or mechanical stress (Bauer et al. (2011), supra), thus longitudinal mechanical measurements appeared to be more robust parameters to investigate in patients with cardiac diseases (Park et al. (2006) American Journal of Physiology—Heart and Circulatory Physiology, supra).

BT2 Treatment Increased Genes Expression Involving Fatty Acids Oxidation

Although contribution of BCAA catabolism to myocardial energetics is limited (Doenst et al. (2013) Circulation Research 113:709-724; Brand et al. (1981) Biochimica et Biophysica Acta (BBA)—General Subjects 677:126-132; Kolwicz et al. (2013) Circulation Research 113:603-616), it has been revealed that disruptive BCAA catabolism suppressed glucose oxidation by inhibiting pyruvate dehydrogenase (PDH) activity and promotes fatty acid oxidation (Li et al. (2017) Cell Metabolism 25:374-385) and affected global metabolic changes (Sun et al. (2016) Circulation 133:2038-2049), indicating the essential roles of BCAA metabolism in regulating cardiac substrate metabolism. To investigate the molecular mechanisms responsible for the therapeutic benefits by BT2, the level of key makers associated with cardiac metabolic synthesis and substrates utilization were measured. Mitochondrial biogenesis seemed unchanged since mitochondrial DNA (mtDNA) (FIG. 44A) mount and PGC-1a mRNA levels (FIG. 44B) remained unaffected between vehicle and BT2 groups post-sham or post-TAC. Interestingly, although gene expression related to glucose transport was unaffected, PDK4 mRNA level showed a trend of increase in BT2 treated mouse heart. (FIG. 44C). This increased trend may indicate decreased glucose catabolism via PDK4 inhibiting regulation on pyruvate dehydrogenase complex (PDC). Moreover, mRNA level of CD36, a marker of fatty acid uptake, moderately increased in BT2 treated mouse heart post-TAC (FIG. 44D). In addition, genes related to fatty acid β-oxidation (Acox1, Acadm) (FIG. 44D) also showed a mild increase in BT2 treated mouse hearts. These data suggested that, in vivo, BCKDK inhibitory therapy appeared to inhibit glucose metabolism and increase fatty acids utilization.

BT2 Treatment Showed No Signs of Toxicity on Mouse Cardiac Function

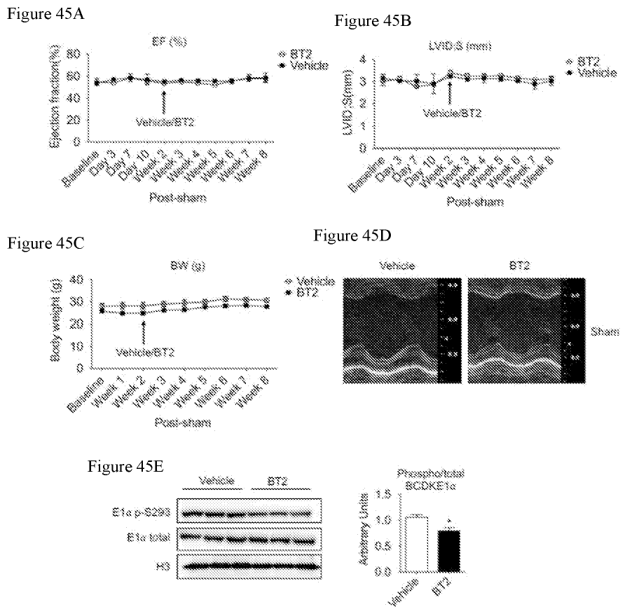

In clinical trials, drug toxicity and safety issues have become a major concern of treating cardiovascular disease. Therefore, evaluating the potential risks of each medication should be considered before initiation. To verify whether BT2 treatment resulted in toxic effects on normal heart function, animals were subjected to sham operation for 2 weeks, and then were randomized to receive vehicle or BT2 compound for additional 6 weeks by daily oral gavage. Cardiac function was assessed by echocardiography before and 8 weeks post-sham operation. Echocardiographic measurements showed that BT2 treatment didn't affect mice systolic function and myocardial structure as no significant changes were detected in EF and LVIDs between this two groups (FIG. 45A and FIG. 45B). Furthermore, mice body weight was not affected by BT2 treatment during the observation periods (FIG. 45C). Together, these data indicated that BT2 treatment had no toxicity on mice normal heart function and body weight, which suggested BT2 would be an ideal drug to further test its therapeutic effect on cardiac dysfunction since no major side effects were observed in this study.

### REFERENCES

1. Gropler et al. (2010) J Nucl Med. 51(suppl 1):88S-101S.
2. van Bilsen et al. (2004) Cardiovasc Res. 61:218-226.
3. van Bilsen et al. (2009) Cardiovasc Res. 81:420-428.
4. Tuunanen and Knuuti (2011) Cardiovasc Res. 90:251-257.
5. Des Rosiers et al. (20011) Cardiovasc Res. 90:210-219.
6. Taegtmeyer et al. (2008) Am J Cardiol. 101:S3-S7.
7. Soukoulis et al. (2009) J Am Coll Cardiol. 54:1660-1673.
8. Peterson et al. (1973) J Mot Cell Cardiol. 5:139-147.
9. Venturini et al. (2009) Mol Cell Biochem. 330:63-70.
10. Kato et al. (20010) Circ Heart Fail. 3:420-430.
11. Lai et al. (2014) Circ Heart Fail. 7:1022-1031.
12. Sansbury et al. (2014) Circ Heart Fail. 7:634-642.
13. Fisch et al. (2007) Proc Natl Acad Sci USA. 104:7074-7079.
14. Haldar et al. (2010) Sci Transl Med. 2:26ra26.
15. Prosdocimo et al. (2014) J Blot Chem. 289:5914-5924.
16. Lu et al. (2009) J Clin Invest. 119:1678-1687.
17. Gao et al. (2016) J Clin Invest. 126:195-206.
18. Tso et al. (2014)J Biol Chem. 289:20583-20593.
19. Olson et al. (2013) Anal Biochem. 439:116-122.
20. Jaswal et al. Molecular Changes in Fatty Acid Oxidation in the Failing Heart Molecular Defects in Cardiovascular Disease. Dhalla N S, Nagano M, Ostadal B, eds. New York: Springer 2011:153-175.
21. Taegtmeyer et al. (2010) Ann N Y Acad Sci. 1188:191-198.
22. Kolwicz and Tian (2011) Cardiovasc Res. 90:194-201.
23. Lee et al. (2011) Circ Res. 109:1332-1341.
24. Huang et al. (2008) Nat Protocols. 4:44-57.
25. Shimizu et al. (2011) Cell Metab. 13:170-182.
26. Gray et al. (2007) Cell Metab. 5:305-312.
27. Chaillou et al. (2013) J Appl Physiol (1985). 115:1065-1074.
28. Zhou et al. (2012) J Biol Chem. 287:23397-23406.
29. Kim et al. (2013) Mol Cells. 35:463-473.
30. Dodd et al. (2012) Am J Physiol Endocrinol Metab. 302:E1329-E1342.
31. Melnik (2012) World J Diabetes. 3:38-53.
32. Vary and Lynch (2007) J Nutr. 137:1835-1843.
33. D'Antona et al. (2010) Cell Metab. 12:362-372.
34. Barschak et al. (2008) Clin Biochem. 41:317-324.
35. Mochel et al. (2007) PLoS One. 2:e647.
36. Shah et al. (2010) Circ Cardiovasc Genet. 3:207-214.
37. Newgard et al. (2009) Cell Metab. 9:311-326.
38. Cheng et al. (2012) Circulation. 125:2222-2231.
39. Mayers et al. (2014) Nat Med. 20:1193-1198.
40. Novarino et al. (2012) Science. 338:394-397.
41. Turer et al. (2009) Circulation. 119:1736-1746.
42. Shah et al. (2009) Mol Syst Biot. 5:258.
43. Héliès-Toussaint et al. (2005) Am J Physiol Regul Integr Comp Physiol. 288:R1325-R1331.
44. Leenders et al. (2010) J Biol Chem. 285:27449-27456.
45. Wang et al. (2008) J Mol Cell Cardiol. 45:193-197.
46. Horie et al. (2009) Biochem Biophys Res Commun. 389:315-320.
47. Gray et al. (2002) J Biol Chem. 277:34322-34328.
48. Mori et al. (2005)J Biol Chem. 280:12867-12875.
49. Nagare et al. (2011)J Biol Chem. 286:37458-37469.
50. Jeyaraj et al. (2012) Cell Metab. 15:311-323.
51. Haldar et al. (2012) Proc Natl Acad Sci USA. 109:6739-6744.
52. Haldar et al. (2010) Sci Transl Med. 2:26ra26.
53. Fisch et al. (2007) Proc Natl Acad Sci USA. 104:7074-7079.
54. Lynch and Adams (2014) Nat Rev Endocrinol. 10:723-736.
55. Tso et al. (2013) Proc Natl Acad Sci USA. 110:9728-9733.
56. Tanada et al. (2015) Life Sci. 137:20-27.
57. Korge et al. (2011)J Biol Chem. 286:34851-34857.
58. Lee et al. (2011) Circulation Research. 109:1332-1341.
59. Fisch et al. (2007) Proc Natl Acad Sci USA. 104:7074-9.
60. Prosdocimo et al. (2014) The Journal of biological chemistry. 289:5914-24.
61. Chokshi et al. (2012) Circulation. 125:2844-53.
62. Lynch et al. (1995) The Biochemical journal. 310 (Pt 1):197-202.
63. Wilson et al. (2011) American journal of physiology Endocrinology and metabolism. 301:E1236-42.
64. Olson et al. (2013) Anal Biochem. 439:116-22.
65. Burrage et al. (2014) Hum Mol Genet 23, R1-8.
66. Felig et al. (1969) New England Journal of Medicine 281, 811-816.
67. Wang et al. (2011) Nat Med 17, 448-453.
68. Lu et al. (2013) Front. Med. 7, 53-59.
69. Shah et al. (2012) Diabetologia 55, 321-330
70. Laferrere et al. (2011) Sci Transl Med 3, 80re82
71. White et al. (2016) Molecular Metabolism 5, 538-551
72. Pedersen et al. (2016) Nature 535:376-381
73. Ridaura et al. (2013) Science 341, DOI: 10.1126/science.1241214
74. Newgard (2012) Cell Metabolism 15, 606-614
75. Lumeng and Saltiel (2011) The Journal of Clinical Investigation 121, 2111-2117
76. Donath (2014) Nat Rev Drug Discov 13, 465-476
77. Ohashi et al. (2014) Trends in Endocrinology & Metabolism 25, 348-355
78. Ouchi et al. (2010) Science 329, 454-457
79. Pietiläinen et al. (2008) PLoS Med 5, e51
80. Lackey et al. (2013) American Journal of Physiology—Endocrinology And Metabolism 304, E1175-E1187
81. She et al. (2007) Am J Physiol Endocrinol Metab 293, E1552-1563
82. Herman et al. (2010) Journal of Biological Chemistry 285, 11348-11356
83. Zimmerman et al. (2013) Molecular Genetics and Metabolism 109, 345-353
84. Shu et al. (2016) bioRxiv doi: https://doi.org/10.1101/036012
85. Bennett et al. (2010) Genome research 20, 281-290
86. Parks et al. (2013) Cell Metab 17, 141-152
87. Lu et al. (2016) Diabetologia 59, 2349-2359
88. Chen et al. (2016) Sci Rep 6, 20594
89. She et al. (2013) PLoS ONE 8, e59443
90. Menni et al. (2013) Diabetes, 62:4270-4276
91. Mori et al. (2012) The Journal of Clinical Investigation 122, 2405-2416
92. Wang et al. (2014) Journal of Molecular Endocrinology 53, 405-415
93. Ajala et al. (2013) The American Journal of Clinical Nutrition 97, 505-516
94. Khazrai et al. (2014) Diabetes/Metabolism Research and Reviews 30, 24-33
95. Brunetti-Pierri et al. (2011) Human Molecular Genetics, 20: 631-640.
96. Mayers et al. (2016) Science. 353:1161-1165.
97. Burrage et al. (2014) Mol. Genet. Metabol. 113:131-135.
98. Kettunen et al. (2012) Nat. Genet. 44:269-276.
99. Xu et al. (2013) Circulation 127:1283-1289.
100. Jang et al. (2016) Nat Med. 22:421-426.
101. Lee et al. (2011) Circulation Research 109:1332-1341.
102. Tso et al. (2014) Journal of Biological Chemistry 289:20583-20593.
103. Bhan et al. (2014) American Journal of Physiology—Heart and Circulatory Physiology 306:H1371-H1383.
104. Schnelle et al. (2017) Journal of Molecular and Cellular Cardiology 114:20-28.
105. Sun et al. (2016) Circulation 133:2038-2049.
106. Li et al. (2017) Cell Metabolism 25:374-385.
107. Wang et al. (2016) American Journal of Physiology—Heart and Circulatory Physiology 311:H1160-H1169.
108. Bijnens et al. (2009) European Journal of Echocardiography 10:216-226.
109. Bauer et al. (2011) Circulation Research 108:908-916.
110. Ternacle et al. (2013) European Heart Journal—Cardiovascular Imaging 14:77-84.
111. Smiseth et al. (2016) European Heart Journal 37:1196-1207.
112. Lumens et al. (2015) JACC: Cardiovascular Imaging 8:1360-1363.
113. Adamo et al. (2017) Circulation: Heart Failure 10.
114. Tower-Rader et al. (2017) Journal of the American Heart Association 6.
115. Wang et al. (2007) Circulation 115:1376-1383.
116. Ersboll et al. (2014) European Heart Journal 35:648-656.
117. Flachskampf et al. (2015) JACC: Cardiovascular Imaging 8:1071-1093.
118. Park et al. (2006) American Journal of Physiology—Heart and Circulatory Physiology 290:H724-H731.
119. Park et al. (2009) Journal of the American Society of Echocardiography 22:183-189.
120. Doenst et al. (2013) Circulation Research 113:709-724.
121. Brand et al. (1981) Biochimica et Biophysica Acta (BBA) —General Subjects 677:126-132.
122. Kolwicz et al. (2013) Circulation Research 113:603-616.
123. Li et al. (2017) Cell Metabolism 25:374-385.
124. Sun et al. (2016) Circulation 133:2038-2049.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the World Wide Web at tigr.org and/or the National Center for Biotechnology Information (NCBI) on the World Wide Web at ncbi.nlm.nih.gov.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of assessing metabolic competence in a subject, the method comprising:
a) administering to the subject a dosage of a branched-chain amino acid (BCAA) and/or a branched-chain α-keto acid (BCKA), wherein the dosage is effective to increase the amount of the BCAA and/or BCKA in the subject;
b) after a pre-determined period of time, obtaining a sample from the subject;
c) measuring the amount of the BCAA and/or BCKA in at least one subject sample; and
d) comparing the measured amount of the BCAA and/or BCKA against a control sample or a pre-determined reference value,
wherein an increase in the amount of the BCAA and/or BCKA in the at least one subject sample relative to the control sample or pre-determined reference value indicates diminished metabolic competence in the subject.

2. (canceled)

3. The method of claim 1, wherein the pre-determined reference value of the BCAA and/or BCKA is a normal physiological level of BCAA and/or BCKA.

4-5. (canceled)

6. A method of prognosing metabolic competence in a subject, the method comprising:
a) administering to the subject a dosage of a branched-chain amino acid (BCAA) and/or a branched-chain α-keto acid (BCKA), wherein the dosage is effective to increase the amount of the BCAA and/or BCKA in the subject;
b) after a pre-determined period of time, obtaining a sample from the subject;
c) measuring the amount of the BCAA and/or BCKA in the sample;
d) comparing the measured amount of the BCAA and/or BCKA in the sample to a control sample or a pre-determined reference value;
wherein an increase in the measured amount of the BCAA and/or BCKA in the at least one subject sample relative to the control sample or pre-determined reference value indicates a positive prognosis for glucose intolerance in the subject.

7. (canceled)

8. A method of assessing the efficacy of an agent for treating a metabolic disorder in a subject, comprising:
a) measuring the levels of a branched-chain amino acid (BCAA) and/or a branched-chain α-keto acid (BCKA) in a first subject sample maintained in the absence of the agent;
b) measuring the levels of the BCAA and/or BCKA in at least one subsequent subject sample contacted with the agent or maintained in the presence of the agent; and
c) comparing the amounts of BCAA and/or BCKA from steps a) and b),
wherein a decrease in the amount in b) relative to the amount in a) indicates that the agent treats the metabolic disorder in the subject.

9-10. (canceled)

11. The method of claim 8, wherein at least one of the subject samples is ex vivo or in vivo sample.

12-13. (canceled)

14. A method for treating or preventing a metabolic disorder in a subject comprising administering to the subject a therapeutically effective amount of at least one agent capable of antagonizing BCKDK, to thereby treat or prevent the metabolic disorder in the subject.

15. The method of claim 14, further comprising administering to the subject an additional agent and/or therapy to treat or prevent the metabolic disorder in the subject.

16. The method of claim 15, wherein the additional therapy comprises a BCAA-deficient diet.

17. The method of claim 1, wherein the subject has glucose intolerance.

18. The method of claim 1, wherein the subject has insulin resistance.

19. The method of claim 1, wherein the subject has obesity, a cancer, and/or a cardiovascular disease, optionally wherein the cardiovascular disease is heart failure.

20. The method of claim 8, wherein the agent antagonizes the expression and/or function of the BCKD kinase (BCKDK).

21. The method of claim 8, wherein the agent increases the expression and/or function of branched-chain alpha-keto acid dehydrogenase (BCKD).

22. The method of claim 8, wherein the agent increases the expression and/or function of Sfrp5.

23. The method of claim 8, wherein the agent is a polynucleotide, a polypeptide, a small molecule compound, or a derivative and/or fragment thereof.

24. The method of claim 23, wherein the polynucleotide is an mRNA or a cDNA.

25. The method of claim 23, wherein the agent is RNA interfering agent.

26-27. (canceled)

28. The method of claim 23, wherein the agent is an antibody or antigen-binding fragment thereof.

29. The method of claim 23, wherein the agent is a small molecule compound that inhibits BCKDK.

30. The method of claim 29, wherein the agent is BT-2.
